(19) United States
(12) Patent Application Publication
(10) Pub. No.: US 2018/0111993 A1
(43) Pub. Date: Apr. 26, 2018

(54) NUCLEIC ACID CONSTRUCT FOR EXPRESSING MORE THAN ONE CHIMERIC ANTIGEN RECEPTOR

(71) Applicant: UCL BUSINESS PLC, London (GB)

(72) Inventors: Martin Pulé, London (GB); Shaun Cordoba, London (GB)

(21) Appl. No.: 15/568,881

(22) PCT Filed: Apr. 26, 2016
(86) PCT No.: PCT/GB2016/051163

§ 371 Date: Oct. 24, 2017

(30) Foreign Application Priority Data
Apr. 27, 2015 (GB) 1507119.4

(51) Int. Cl. C07K 16/28; A61K 35/17; C07K 14/705; C12N 9/16; A61P 35/00; C07K 16/30; C12N 7/00

(52) U.S. Cl. CPC C07K 16/2803; C12N 2710/10333; C07K 14/70517; C07K 14/70521; C12N 9/16; C12Y 301/03048; C07K 14/70503; A61P 35/00; C07K 16/2863; C07K 16/3084; C12N 7/00; C07K 2317/622; C07K 2319/02; C07K 2319/03; C07K 2319/04; C07K 2319/05; A61K 35/17

(57) ABSTRACT

The present invention provides a nucleic acid construct comprising the following structure: A-X-B in which X is a nucleic acid sequence which encodes a cleavage site; and A and B are nucleic acid sequences encoding a first and a second chimeric antigen receptor (CAR), each CAR comprising: (i) an antigen-binding domain; (ii) a spacer (iii) a trans-membrane domain; and (iv) an endodomain wherein the antigen binding domains of the first and second CARs bind to different antigens; wherein one of the first or second CARs is an activating CAR comprising an activating endodomain and the other CAR is an inhibitory CAR comprising a ligation-off inhibitory endodomain; and wherein: (a) the first and/or second CAR comprises an intracellular retention signal; and/or (b) the signal peptide of the first or second CAR comprises one or more mutation(s) such that it has fewer hydrophobic amino acids.

### FIELD OF THE INVENTION

The present invention relates to constructs and approaches for expressing more than one chimeric antigen receptor (CAR) at the surface of a cell. The cell may be capable of specifically recognising a target cell, due to a differential pattern of expression (or non-expression) of two or more antigens by the target cell. The constructs of the invention enable modulation of the relative expression of the two or more CARs at the cell surface by a method involving co-expression of the CARs from a single vector.

### BACKGROUND TO THE INVENTION

A number of immunotherapeutic agents have been described for use in cancer treatment, including therapeutic monoclonal antibodies (mAbs), immunoconjugated mAbs, radioconjugated mAbs and bi-specific T-cell engagers.

Typically these immunotherapeutic agents target a single antigen: for instance, Rituximab targets CD20; Myelotarg targets CD33; and Alemtuzumab targets CD52.

However, it is relatively rare for the presence (or absence) of a single antigen effectively to describe a cancer, which can lead to a lack of specificity. Targeting antigen expression on normal cells leads to on-target, off-tumour toxicity.

Most cancers cannot be differentiated from normal tissues on the basis of a single antigen. Hence, considerable “on-target off-tumour” toxicity occurs whereby normal tissues are damaged by the therapy. For instance, whilst targeting CD20 to treat B-cell lymphomas with Rituximab, the entire normal B-cell compartment is depleted, whilst targeting CD52 to treat chronic lymphocytic leukaemia, the entire lymphoid compartment is depleted, whilst targeting CD33 to treat acute myeloid leukaemia, the entire myeloid compartment is damaged etc.

The predicted problem of “on-target off-tumour” toxicity has been bourne out by clinical trials. For example, an approach targeting ERBB2 caused death to a patient with colon cancer metastatic to the lungs and liver. ERBB2 is over-expressed in colon cancer in some patients, but it is also expressed on several normal tissues, including heart and normal vasculature.

In some cancers, a tumour is best defined by presence of one antigen (typically a tissue-specific antigen) and the absence of another antigen which is present on normal cells. For example, acute myeloid leukaemia (AML) cells express CD33. Normal stem cells express CD33 but also express CD34, while AML cells are typically CD34 negative. Targeting CD33 alone to treat AML is associated with significant toxicity as it depletes normal stem cells. However, specifically targeting cells which are CD33 positive but not CD34 positive would avoid this considerable off-target toxicity.

There is thus a need for immunotherapeutic agents which are capable of more targeting to reflect the complex pattern of marker expression that is associated with many cancers.

Chimeric Antigen Receptors (CARs)

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

However, the use of CAR-expressing T cells is also associated with on-target, off tumour toxicity. For example, a CAR-based approach targeting carboxy anyhydrase-IX (CAIX) to treat renal cell carcinoma resulted in liver toxicity which is thought to be caused by the specific attack on bile duct epithelial cells (Lamers et al (2013) Mol. Ther. 21:904-912.

There is thus a need for alternative CAR-based T cell approaches with greater selectivity and reduced on target, off tumour toxicity.

### DESCRIPTION OF THE FIGURES

FIG. 1: (a) Generalized architecture of a CAR: A binding domain recognizes antigen; the spacer elevates the binding domain from the cell surface; the trans-membrane domain anchors the protein to the membrane and the endodomain transmits signals. (b) to (d): Different generations and permutations of CAR endodomains: (b) initial designs transmitted ITAM signals alone through FcϵR1-γ or CD3ζ endodomain, while later designs transmitted additional (c) one or (d) two co-stimulatory signals in cis.

FIG. 2: Schematic diagram illustrating the invention

The invention relates to engineering T-cells to respond to logical rules of target cell antigen expression. This is best illustrated with an imaginary FACS scatter-plot. Target cell populations express both, either or neither of antigens “A” and “B”. Different target populations (marked in red) are killed by T-cells transduced with a pair of CARs connected by different gates. With OR gated receptors, both single-positive and double-positive cells will be killed. With AND gated receptors, only double-positive target cells are killed. With AND NOT gating, double-positive targets are preserved while single-positive targets

FIG. 3: Creation of target cell populations

SupT1 cells were used as target cells. These cells were transduced to express either CD19, CD33 or both CD19 and CD33. Target cells were stained with appropriate antibodies and analysed by flow cytometry.

FIG. 4: Cartoon showing three versions of the cassette used to generate the AND NOT gate

Activating and inhibiting CARs were co-expressed once again using a FMD-2A sequence. Signal1 is a signal peptide derived from IgG1 (but can be any effective signal peptide). scFv1 is the single-chain variable segment which recognizes CD19 (but can be a scFv or peptide loop or ligand or in fact any domain which recognizes any desired arbitrary target). STK is the human CD8 stalk but may be any non-bulky extracellular domain. CD28tm is the CD28 trans-membrane domain but can by any stable type I protein transmembrane domain and CD3Z is the CD3 Zeta endodomain but can be any endodomain which contains ITAMs. Signal2 is a signal peptide derived from CD8 but can be any effective signal peptide which is different in DNA sequence from signal1. scFv recognizes CD33 but as for scFv1 is arbitrary. muSTK is the mouse CD8 stalk but can be any spacer which co-localises but does not cross-pair with that of the activating CAR. dPTPN6 is the phosphatase domain of PTPN6. LAIR1 is the transmembrane and endodomain of LAIR1. 2Aw is a codon-wobbled version of the FMD-2A sequence. SH2-CD148 is the SH2 domain of PTPN6 fused with the phosphatase domain of CD148.

FIG. 5: Schematic representation of the chimeric antigen receptors (CARs) for the NOT AND gates
A) A stimulatory CAR consisting of an N-terminal anti-CD19 scFv domain followed by the stalk region of human CD8, human CD28 transmembrane domain and human CD247 intracellular domain. B) An inhibitory CAR consisting of an N-terminal anti-CD33 scFv domain followed by the stalk region of mouse CD8, transmembrane region of mouse CD8 and the phosphatase domain of PTPN6. C) an inhibitory CAR consisting of an N-terminal anti-CD33 scFv domain followed by the stalk region of mouse CD8 and the transmembrane and intracellular segments of LAIR1. D) An inhibitory CAR identical to previous CAR except it is co-expressed with a fusion protein of the PTPN6 SH2 domain and the CD148 phosphatase domain.

FIG. 6: Functional analysis of the NOT AND gate

Effector cells (5×10̂4 cells) expressing the A) full length SHP-1 or B) truncated form of SHP-1 were co-incubated with a varying number of target cells and IL-2 was analysed after 16 hours by ELISA. The graph displays the average maximum IL-2 secretion from a chemical stimulation (PMA and lonomycin) of the effector cells alone and the average background IL-2 from effector cells without any stimulus from three replicates.

FIG. 7: Function of the AND NOT gates

Function of the three implementations of an AND NOT gate is shown. A cartoon of the gates tested is shown to the right, and function in response to single positive and double positive targets is shown to the left. A. PTPN6 based AND NOT gate whereby the first CAR recognizes CD19, has a human CD8 stalk spacer and an ITAM containing activating endodomain; is co-expressed with a second CAR that recognizes CD33, has a mouse CD8 stalk spacer and has an endodomain comprising of a PTPN6 phosphatase domain. B. ITIM based AND NOT gate is identical to the PTPN6 gate, except the endodomain is replaced by the endodomain from LAIR1. C. CD148 boosted AND NOT gate is identical to the ITIM based gate except an additional fusion between the PTPN6 SH2 and the endodomain of CD148 is expressed. All three gates work as expected with activation in response to CD19 but not in response to CD19 and CD33 together.

FIG. 8: Dissection of PTPN6 based AND NOT gate function

The original PTPN6 based AND NOT gate is compared with several controls to demonstrate the model. A cartoon of the gates tested is shown to the right, and function in response to single positive and double positive targets is shown to the left. A. Original AND NOT gate whereby the first CAR recognizes CD19, has a human CD8 stalk spacer and an ITAM containing activating endodomain; is co-expressed with a second CAR recognizes CD33, has a mouse CD8 stalk spacer and has an endodomain comprising of a PTPN6 phosphatase domain. B. AND NOT gate modified so the mouse CD8 stalk spacer is replaced with an Fc spacer. C AND NOT gate modified so that the PTPN6 phosphatase domain is replaced with the endodomain from CD148. Original AND NOT gate (A.) functions as expected triggering in response to CD19, but not in response to both CD19 and CD33. The gate in B. triggers both in response to CD19 along or CD19 and CD33 together. The gate in C. does not trigger in response to one or both targets.

FIG. 9: Dissection of LAIR1 based AND NOT gate

Functional activity against CD19 positive, CD33 positive and CD19, CD33 double-positive targets is shown. A. Structure and activity of the original ITIM based AND NOT gate. This gate is composed of two CARs: the first recognizes CD19, has a human CD8 stalk spacer and an ITAM containing endodomain; the second CAR recognizes CD33, has a mouse CD8 stalk spacer and an ITIM containing endodomain. B Structure and activity of the control ITIM based gate where the mouse CD8 stalk spacer has been replaced by an Fc domain. This gate is composed of two CARs: the first recognizes CD19, has a human CD8 stalk spacer and an ITAM containing endodomain; the second CAR recognizes CD33, has an Fc spacer and an ITIM containing endodomain. Both gates respond to CD19 single positive targets, while only the original gate is inactive in response to CD19 and CD33 double positive targets.

FIG. 10: Kinetic segregation model of CAR logic gates

Model of kinetic segregation and behaviour of AND gate, NOT AND gate and controls. CARs recognize either CD19 or CD33. The immunological synapse can be imagined between the blue line, which represents the target cell membrane and the red line, which represents the T-cell membrane. ‘45’ is the native CD45 protein present on T-cells. ‘H8’ is a CAR ectodomain with human CD8 stalk as the spacer. ‘Fc’ is a CAR ectodomain with human HCH2CH3 as the spacer. ‘M8’ is a CAR ectodomain with murine CD8 stalk as the spacer. ‘19’ represents CD19 on the target cell surface. ‘33’ represents CD33 on the target cell surface. The symbol ‘⊕’ represents an activating endodomain containing ITAMS. The symbol ‘⊖’ represents a phosphatase with slow kinetics—a ‘ligation on’ endodomain such as one comprising of the catalytic domain of PTPN6 or an ITIM. The symbol ‘Ø’ represents a phosphatase with fast kinetics—a ‘ligation off’ endodomain such as the endodomain of CD45 or CD148. This symbol is enlarged in the figure to emphasize its potent activity.

(a) Shows the postulated behaviour of the functional AND gate which comprises of a pair of CARs whereby the first CAR recognizes CD19, has a human CD8 stalk spacer and an activating endodomain; and the second CAR recognizes CD33, has an Fc spacer and a CD148 endodomain;
(b) Shows the postulated behaviour of the control AND gate. Here, the first CAR recognizes CD19, has a human CD8 stalk spacer and an activating endodomain; and the second CAR recognizes CD33, but has a mouse CD8 stalk spacer and a CD148 endodomain;
(c) Shows the behaviour of a functional AND NOT gate which comprises of a pair of CARs whereby the first CAR recognizes CD19, has a human CD8 stalk spacer and an activating endodomain; and the second CAR recognizes CD33, has a mouse CD8 stalk spacer and a PTPN6 endodomain;
(d) Shows the postulated behaviour of the control AND NOT gate which comprises of a pair of CARs whereby the first CAR recognizes CD19, has a human CD8 stalk spacer and an activating endodomain; and the second CAR recognizes CD33, but has an Fc spacer and a PTPN6 endodomain;

In the first column, target cells are both CD19 and CD33 negative. In the second column, targets are CD19 negative and CD33 positive. In the third column, target cells are CD19 positive and CD33 negative. In the fourth column, target cells are positive for both CD19 and CD33.

FIG. 11: IgM and IgG in ANDNOT gate

To test if the ANDNOT gate could function on extended spacer lengths, both the activating CAR (anti-CD19) and the inhibiting CAR (anti-CD33) spacers were substituted for longer spacers. The Fc region of human IgM and IgG were used to extend the spacer length. The Fc of IgM contains and additional Ig domain compared to IgG, for this reason the IgM spacer was placed on the anti-CD19 CAR which is known to have a membrane proximal binding epitope. In contrast the anti-CD33 binding epitope is located on a distal end of the molecule, thus the relatively shorter IgG spacer was used on this CAR. The extended spacer ANDNOT gate construct was transduced into a mouse T-cell line. Then a fixed number of transduced T-cells were co-cultured with a varying number of target cells for 16-24 hours, after which the amount of IL-2 secreted in the supernatant was analysed via ELISA.

FIG. 12: Anti-CD19/anti-GD2 ANDNOT gate:

To test the robustness of the ANDNOT gate platform, the binding domain from the inhibitory CAR (anti-CD33) was substituted with two other unrelated binders (anti-GD2 and anti-EGFRvIII). The scFv fragment for anti-GD2 or anti-EGFRvIII was substituted for anti-CD33 on the inhibitory CAR in the ANDNOT gate platform with either a truncated SHP-1 or LAIR cytosolic domain. These constructs were transduced into a mouse T-cell line and a fixed number of T-cells were co-cultured with a varying number of target cells. After 16-24 hours of co-culture the amount of IL-2 secreted in the supernatant was analysed via ELISA. A) Anti-CD19/anti-GD2 ANDNOT gate B) Anti-CD19/anti-EGFRvIII ANDNOT gate.

FIG. 13: A selection/hierarchy of possible spacer domains of increasing size is shown.

The ectodomain of CD3-Zeta is suggested as the shortest possible spacer, followed by the (b) the IgG1 hinge. (c) murine or human CD8 stalk and the CD28 ectodomains are considered intermediate in size and co-segregate. (d) The hinge, CH2 and CH3 domain of IgG1 is bigger and bulkier, and (e) the hinge, CH2, CH3 and CH4 domain of IgM is bigger still. Given the properties of the target molecules, and the epitope of the binding domains on said target molecules, it is possible to use this hierarchy of spacers to create a CAR signaling system which either co-segregates or segregates apart upon synapse formation.

FIG. 14: Matrix for the AND NOT gate platform.

Multiple AND NOT gates were produced and tested with various combinations of the spacers: hinge; huCD8STK; huCD28ecto; hulgG (HCH2CH3pvaa); and hulgM (CH2HCH3CH4) as described in the legend for FIG. 13. They were tested in the AND NOT gate platform as previously described with a CD19 signalling CAR and a CD33 inhibitory CAR with a CD148 ligation-off inhibitory endodomain. Blue=predicted; Red=proven; NO STIM=no stimulation with any cognate targets; AND=functions as AND NOT gate.

FIG. 15: Methods utilised to express different proteins from the same vector

(a) Two different promoters within the same cassette result in two different transcripts which each give rise to separate proteins. (b) Use of an Internal Ribosome Entry sequence (IRES) leads to a single transcript which is translated into two separate proteins. (c) Use of the FMDV 2A peptide results in a single transcript, and a single polyprotein which rapidly cleaves into two separate proteins.

FIG. 16: TYRP1 endodomain is able to direct the retention of a transmembrane protein with a complex endodomain

Tyrp1 is a type I transmembrane protein, 537aa long. The di-leucine motif, which retains the protein in the intracellular compartment, is indicated as a black rectangle on the cytoplasmic domain. (A) Tyrp1 (wt). Wild type Tyrp1 consists of a peptide signal, a luminal domain, a transmembrane domain, and a cytoplasmic domain. The cytoplasmic domain contains the di-leucine retention signal. (B) Tyrp1 (wt)-SG Linker-eGFP. This construct contains the wild type Tyrp1 simply fused to eGFP via a serine-glycine-glycine-glycine-serine linker. The Tyrp1-L-eGFP represents the cytoplasmic-proximal Tyrp1. (C) Tyrp1 Lumenal (LM)-Transmembrane (TM)-SG Linker-eGFP-Tyrp1 Cytoplasmic (CP). This construct constitutes the cytoplasmic-distal Tyrp1, since SG linker-eGFP interposes between the transmembrane and cytoplasmic domains. D: Tyrp1 Lumenal (LM)-Transmembrane (TM)-SG Linker-eGFP. This construct serves as the positive control, as the cytoplasmic domain containing the retention signal has been excluded. All constructs are co-expressed with IRES.CD34. Staining of transduced SupT1 cells is shown with intracellular and surface staining bottom left/right respectively.

FIG. 17: Functionality of the TYRP1 retention signal in primary cells

A construct was generated which co-express an anti-CD19 and an anti-CD33 CAR using a FMD-2A like peptide. Two variants of this construct were also generated: in the first variant, the di-leucine motif from TYRP1 was inserted into the anti-CD19 CAR endodomain just proximal to the TM domain; In the second variant the same TYRP1 di-lecuine motif was attached to the carboxy-terminus of the anti-CD19 CAR endodomain. PBMCs were isolated from blood and stimulated using PHA and IL-2. Two days later the cells were transduced on retronectin coated plates with retro virus containing the different CD19:CD33 CAR constructs. On day 5 the expression level of the two CARs translated by the construct was evaluated via flow cytometry using recombinant CD19-Fc and CD33-Fc fusions. A. Shows cartoon of the synthetic gene constructed to allow co-expression; B. Shows a cartoon of the subsequent pairs of proteins generated by the three construcs; C. Shows expression of the two receptors by flow-cytometry. In the original construct, both CARs are equally expressed. With incorporation of the di-leucine motif distally in the endodomain of the anti-CD19 CAR, the CD33 CAR expression remains constant but the CD19 expression drops to intermediate levels. With incorporation of the di-leucine motif proximally in the endodomain of the anti-CD19 CAR, the CD33 CAR expression remains constant, but the CD19 expression drops to low levels.

FIG. 18: Retention signal from cytosolic tail of E3/19K

A construct was generated which co-expresses an anti-CD19 and an anti-CD33 CAR using a FMD-2A like peptide. Two variants of this construct were also generated: in the first variant, the last 6aa from E3/19K (DEKKMP), which were found to be critical for its Golgi/ER retention ability, were attached to the carboxy-terminus of the anti-CD33 CAR endodomain; in the second variant, the entire cytosolic tail of adenovirus E3/19K protein was attached to the carboxy-terminus of the anti-CD33 CAR endodomain

FIG. 19: Functionality of E3/19K retention signal

The constructs shown in FIG. 4 were transfected into 293T cells and the expression level of the two CARs translated by the construct was evaluated via flow cytometry using recombinant CD19-Fc and CD33-Fc fusions. A clear retention was observed when the full length adenovirus E3/19K protein, or the DEKKMP motif was placed on the anti-CD33 receptor. The anti-CD19 receptor expression levels were unaffected.

FIG. 20: Schematic diagram illustrating the function of signal sequences in protein targeting

FIG. 21: Schematic diagram of nucleic acid construct encoding two CARs

FIG. 22: Verifying the function of a substituted signal sequence.

PCT/GB2014/053452 describes vector system encoding two chimeric antigen receptors (CARs), one against CD19 and one against CD33. The signal peptide used for the CARs in that study was the signal peptide from the human CD8a signal sequence. For the purposes of this study, this was substituted with the signal peptide from the murine Ig kappa chain V-III region, which has the sequence: METDTLILWVLLLLVPGSTG (hydrophobic residues hightlited in bold). In order to establish that the murine Ig kappa chain V-III signal sequence functioned as well as the signal sequence from human CD8a, a comparative study was performed. For both signal sequences, functional expression of the anti-CD33 CAR and the anti-CD19 CAR was observed.

FIG. 23: Testing the effect of one amino acid deletion in the murine Ig kappa chain V-Ill. Mutant 1 kappa chain was created with the following deletion (shown in grey) in the h-region METD [symbol] LWVLLLLVPGSTG and the relative expression on the anti-CD33 CAR and the anti-CD19 CAR was observed.

FIG. 24: Testing the effect of two amino acid deletions in the murine Ig kappa chain V-Ill. Mutant 2 kappa chain was created with the following deletions (shown in grey) in the h-region METDT [symbol] LWVLLL [symbol] VPGSTG and the relative expression on the anti-CD33 CAR and the anti-CD19 CAR was observed.

FIG. 25: Testing the effect of three amino acid deletions in the murine Ig kappa chain V-Ill. Mutant 2 kappa chain was created with the following deletions (shown in grey) in the h-region METDT [symbol] LWVLLL [symbol] VPGSTG and the relative expression on the anti-CD33 CAR and the anti-CD19 CAR was observed.

FIG. 26: Testing the effect of five amino acid deletions in the murine Ig kappa chain V-Ill. Mutant 2 kappa chain was created with the following deletions (shown in grey) in the h-region METDT [symbol] LWVLLL [symbol] PGSTG and the relative expression on the anti-CD33 CAR and the anti-CD19 CAR was observed.

### SUMMARY OF ASPECTS OF THE INVENTION

The present inventors have developed a panel of “logic-gated” chimeric antigen receptor pairs which, when expressed by a cell, such as a T cell, are capable of detecting a particular pattern of expression of at least two target antigens. If the at least two target antigens are arbitrarily denoted as antigen A and antigen B, the three possible options are as follows:

“OR GATE”—T cell triggers when either antigen A or antigen B is present on the target cell “AND GATE”—T cell triggers only when both antigens A and B are present on the target cell “AND NOT GATE”—T cell triggers if antigen A is present alone on the target cell, but not if both antigens A and B are present on the target cell

Engineered T cells expressing these CAR combinations can be tailored to be exquisitely specific for cancer cells, based on their particular expression (or lack of expression) of two or more markers.

The present inventors have also found that, when a CAR is co-expressed with a second CAR as a polyprotein which after translation is subsequently cleaved to separate the two CARs, it is possible to modulate the relative cell surface expression of the two CARs by reducing trafficking to the cell surface of one or both CAR(s) and/or by reducing its half-life at the cell surface. This need not be limited to a pair of CARs, but may be used to allow control of the relative expression of multiple proteins initially translated as a polyprotein.

As used herein, ‘polyprotein’ refers to a polypeptide sequence translated from a single nucleic acid construct as a single entity, but which comprises polypeptide sequences which are subsequently separated and which function as discrete entities (e.g. separate CARs).

The present invention relates to an AND NOT logic gate, in which the relative expression of the two CARs is modulated.

Thus in a first aspect, the present invention provides a cell which co-expresses a first chimeric antigen receptor (CAR) and second CAR at the cell surface, each CAR comprising:
(i) an antigen-binding domain;
(ii) a spacer
(iii) a trans-membrane domain; and
(iv) an intracellular T cell signaling domain (endodomain)
   wherein the antigen binding domains of the first and second CARs bind to different antigens; wherein one of the first or second CARs is an activating CAR comprising an activating intracellular T cell signaling domain and the other CAR is an inhibitory CAR comprising a “ligation-on” (as defined herein) inhibitory intracellular T cell signaling domain;
   and wherein:
(a) the first and/or second CAR comprises an intracellular retention signal; and/or
(b) the signal peptide of the first or second CAR comprises one or more mutation(s) such that it has fewer hydrophobic amino acids.

For (b) the mutated signal peptide may have fewer hydrophobic amino acids than the “wild-type” signal peptide sequence from which it is derived. The mutated signal peptide may have fewer hydrophobic amino acids than the signal peptide of the other CAR.

The spacer of the first CAR may be different to the spacer of the second CAR.

The spacers of the first and second CARs may be sufficiently different as to prevent cross-pairing, but to be sufficiently similar to cause the CARs to co-localise at the T cell membrane.

The spacers of the first and second CARs may be orthologous, such as mouse and human CD8 stalks.

In the present invention, which relates to the “AND NOT” gate, one of the first or second CARs is an activating CAR comprising an activating endodomain, and the other CAR is an inhibitory CAR comprising a “ligation-on” inhibitory endodomain. The inhibitory CAR does not significantly inhibit T-cell activation by the activating CAR in the absence of inhibitory CAR ligation, but inhibits T-cell activation by the activating CAR when the inhibitory CAR is ligated. In these embodiments, the first and second spacers are sufficiently different so as to prevent cross-pairing of the first and second CARs but are sufficiently similar to result in co-localisation of the first and second CARs following ligation.

The inhibitory endodomain may comprise at least part of a protein-tyrosine phosphatase.

The inhibitory endodomain may comprise all or part of PTPN6.

The inhibitory endodomain may comprise an ITIM domain.

The inhibitory endodomain may comprise an ITIM domain in conjunction with co-expression of a fusion between at least part of a protein-tyrosine phosphatase and at least part of a receptor-like tyrosine phosphatase. The fusion may comprise one or more SH2 domains from the protein-tyrosine phosphatase. For example, the fusion may be between a PTPN6 SH2 domain and CD45 endodomain or between a PTPN6 SH2 domain and CD148 endodomain.

As explained in the introduction, acute myeloid leukaemia (AML) cells express CD33. Normal stem cells express CD33 but also express CD34, while AML cells are typically CD34 negative. Targeting CD33 alone to treat AML is associated with significant toxicity as it depletes normal stem cells. However, specifically targeting cells which are CD33 positive but not CD34 positive avoids this considerable off-target toxicity. Thus in the present invention, the CAR comprising the activating endodomain may comprise an antigen-binding domain which binds CD33 and the CAR which comprises the ligation-on inhibitory endodomain may comprise an antigen-binding domain which binds CD34.

The present invention relates to a method for modulating the relative expression of two (or more) CARs in an AND NOT logic gate, by one or both of the following approaches (a) by incorporation of an intracellular retention signal in one or both CAR(s) and (b) by altering the signal peptide of one CAR in order to remove or replace hydrophobic amino acids.

In particular, the relative expression level of the activating CAR, in relation to the inhibitory CAR, may be reduced by one of the above-mentioned approaches.

For approach (a), the endodomain of the CAR may comprise the intracellular retention signal.

The intracellular retention signal may direct the CAR away from the secretory pathway and/or to a membrane-bound intracellular compartment such as a lysozomal, endosomal or Golgi compartment.

The intracellular retention signal may, for example, be a tyrosine-based sorting signal, a dileucine-based sorting signal, an acidic cluster signal, a lysosomal avoidance signal, an NPFX′(1,2)D-Type signal, a KDEL, a KKX′X′ or a KX′KX′X′ signal (wherein X′ is any amino acid).

The intracellular retention signal may comprise a sequence selected from the group of: NPX′Y, YX′X′Z′, [DE]X′X′X′L[LI], DX′X′LL, DP[FW], FX′DX′F, NPF, LZX′Z[DE], LLDLL, PWDLW, KDEL, KKX′X′ or KX′KX′X′;

wherein X′ is any amino acid and Z′ is an amino acid with a bulky hydrophobic side chain.

The intracellular retention signal may comprise any of the sequences shown in Tables 1 to 5.

The intracellular retention signal may comprise the Tyrosinase-related protein (TYRP)-1 intracellular retention signal. The intracellular retention signal may comprise the TYRP-1 intracellular domain. The intracellular retention signal may comprise the sequence NQPLLTD (SEQ ID No. 1).

The intracellular retention signal may comprise the Adenoviral E3/19K intracellular retention signal. The intracellular retention signal may comprise the E3/19K cytosolic domain. The intracellular retention signal may comprise the sequence KYKSRRSFIDEKKMP (SEQ ID No. 2); or DEKKMP (SEQ ID No. 3).

The intracellular retention signal may be proximal or distal to a transmembrane domain of the CAR.

For approach (b), the signal peptides of the first and second CAR are different. They may differ in their number of hydrophobic amino acids. One signal peptide may comprise one or more mutation(s) such that it has fewer hydrophobic amino acids either a) than the wild-type sequence from which it was derived; or b) than the other signal peptide.

In particular the signal peptide of the activating CAR may be altered to remove or replace hydrophobic amino acids, such that the relative expression of the activating CAR, relative to the inhibitory CAR, is reduced at the cell surface.

The signal peptides may be different and the sequence of one signal peptide may be altered such that the hydrophobic amino acids are removed or replaced. Alternatively, the first signal peptide and the second signal peptide may be derivable from the same sequence, but one signal peptide may comprise one or more amino acid deletions/substitutions to remove/replace one or more hydrophobic amino acids compared to the other signal peptide.

The hydrophobic amino acid(s) removed or replaced may be selected from the group: Alanine (A); Valine (V); Isoleucine (I); Leucine (L); Methionine (M); Phenylalanine (P); Tyrosine (Y); Tryptophan (W) or the group Valine (V); Isoleucine (I); Leucine (L); and Tryptophan (W).

For approach (b) one signal peptide may comprise one, two, three, four or five mutations, such that it has one, two, three, four or five fewer hydrophobic amino acids than: the wild-type signal sequence from which it is derived and/or the other signal peptide.

In the nucleic acid construct of the present invention, the nucleic acid sequence of X may be a nucleic acid sequence encoding a self-cleaving peptide, a furin cleavage site or a Tobacco Etch Virus cleavage site.

The nucleic acid sequence of X may be a nucleic acid sequence encoding a 2A self-cleaving peptide from an aphtho- or a cardiovirus or a 2A-like peptide.

The nucleic acid construct may comprise a third nucleic acid sequence encoding a protein of interest (POI). The POI may be a transmembrane protein.

The POI may be selected from a list of: excitatory receptors such as 41BB, OX40, CD27, CD28 and related molecules; or inhibitory receptors such as PD1, CTLA4, LAIR1, CD22 and related molecules; or cytokine receptor molecules such as IL1R, IL2R, IL7R, IL15R and related molcules; or homing molecules such as N-CAM, V-CAM, L1-CAM, LFA-1, CDH1-3, Selectins or Integrins. The POI may be a third CAR.

The POI may be a synthetic protein such as a suicide gene or a marker gene.

The amount of a CAR which comprises an intracellular retention signal and/or which has an altered signal peptide which is expressed at the cell surface may be, for example, less than 90%, 70%, 50% or 30% compared to a CAR expressed from the same nucleic acid construct which does not comprise an intracellular retention signal and which has an unaltered signal peptide.

The nucleic acid construct may also encode a further polypeptide, for example a polypeptide which enables selection of transduced cells and/or enables cells expressing the polypeptide to be deleted.

Alternative codons may be used in regions of sequence encoding the same or similar amino acid sequences, in order to avoid homologous recombination.

In a second aspect the present invention provides a vector comprising a nucleic acid construct according to the first aspect of the invention.

The vector may be a retroviral vector or a lentiviral vector or a transposon.

In a third aspect the present invention provides a cell comprising a nucleic acid construct according to the first aspect of the invention or a vector according to the second aspect of the invention.

The cell may be an immune cell such as a T cell or a natural killer (NK) cell.

In a fourth aspect, the present invention provides a method for making a cell according to the third aspect of the invention, which comprises the step of introducing: a nucleic acid construct according to the first aspect of the invention or a vector according to the second aspect of the invention, into a cell.

The cell may be part of or derived from a sample isolated from a subject.

The cell used in the method of the fourth aspect of the invention may be from a sample isolated from a patient, a related or unrelated haematopoietic transplant donor, a completely unconnected donor, from cord blood, differentiated from an embryonic cell line, differentiated from an inducible progenitor cell line, or derived from a transformed cell line.

In a fifth aspect the present invention provides a pharmaceutical composition comprising a plurality of cells according to the forth aspect of the invention. The composition may be an autologous T and/or NK cell composition.

In a sixth aspect, the present invention provides a method for treating and/or preventing a disease, which comprises the step of administering a pharmaceutical composition according to the fifth aspect of the invention to a subject.

The method may comprise the following steps:
(i) isolation of a T and/or NK cell-containing sample from a subject;
(ii) transduction or transfection of the T and/or NK cells with: a nucleic acid construct according to the first aspect of the invention or a vector according to the second aspect of the invention; and
(iii) administering the T and/or NK cells from (ii) to a the subject.

There is also provided a pharmaceutical composition according to the fifth aspect of the invention for use in treating and/or preventing a disease.

There is also provided the use of a cell according to the third aspect of the invention in the manufacture of a medicament for treating and/or preventing a disease.

The invention further relates to method for modulating the relative cell surface expression of an activating CAR expressed from a single nucleic acid construct with a ligation-on inhibitory CAR by (a) including an intracellular retention signal in the nucleic acid sequence which encodes the activating CAR(s) and/or (b) altering the nucleic acid sequence which encodes the signal peptide of the activating CAR in order to remove or replace one or more hydrophobic amino acids in comparison with the signal peptide of the ligation-on inhibitory CAR. The nucleic acid construct may be as defined in the first aspect of the invention.

The “AND NOT gate” of the present invention offers a significant advantage over the CAR approaches described to date which involve targeting a single tumour-associated antigen. Here, where a tumour cell is characterized by the presence of one (or more) antigen(s) and the absence of another antigen, this can be specifically targeted using the CAR based AND NOT approach of the present invention. A normal cell, which expressed both antigens will not be targeted, leading to greater selectivity and reduced on target, off tumour toxicity. A CAR approach directed to a single antigen would target both tumour cells and normal cells in this situation.

Moreover the capacity to modulate the relative expression of the two CARs, provided by the present invention, brings further advantages to the AND NOT gate. When an AND NOT gate system is working correctly, the ligation-on inhibitory CAR inhibits T-cell activation by the activating CAR in the presence of inhibitory CAR ligation, but does not significantly inhibit T-cell activation by the activating CAR when the inhibitory CAR is unligated. However, in some systems the activating CAR is “overactive” leading to a high level of background i.e. activation by the activating CAR in the presence of inhibitory CAR ligation. The present inventors have found that, by down regulating the relative expression of the activating CAR in comparison to the inhibitory CAR, it is possible to “tighten up” the system and reduce the level of background activation in the presence of the second antigen.

The inclusion of an intracellular retention signal in a CAR, or the alteration of the signal peptide of the CAR to reduce the number of hydrophobic amino acids, reduces the amount of the CAR expressed on the cell surface. As such, the relative expression level of two CARs expressed from a single construct can be modulated. As a CAR is only active at the cell surface, reducing the relative cell surface expression of the CAR also reduces its relative activity.

Thus the present invention provides a nucleic acid construct encoding an AND NOT gate, in which the relative level of expression of the two or more CARs may be finely tuned, either to reduce the expression of the activating CAR (as described above) or to mirror the relative level of expression of the respective antigens on the non-cancerous cell which expresses both antigens.

This invention can be extended to modulate the relative expression of three or more proteins expressed as a concatenated polypeptide, separated by cleavage sites and relative surface expression dictated by retention signals or signal peptides of differing activity.

### DETAILED DESCRIPTION

Chimeric Antigen Receptors (CARS)

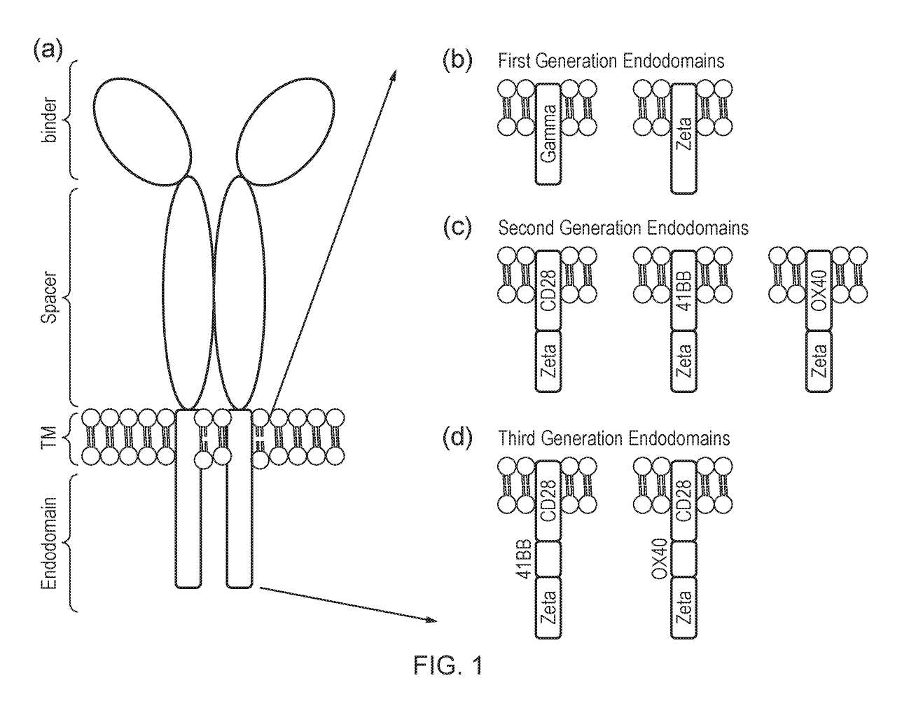

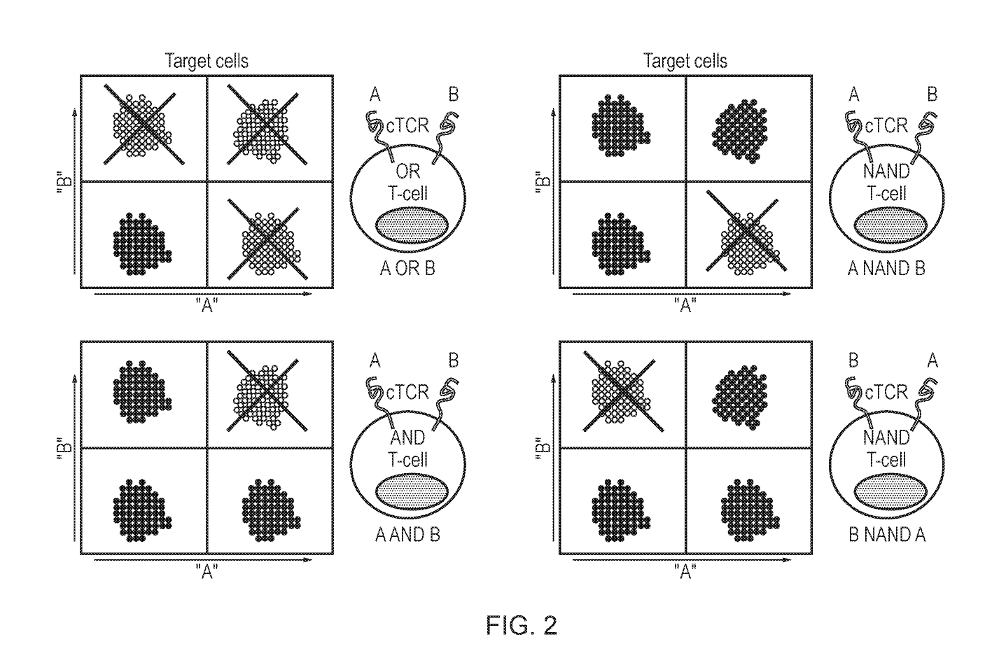

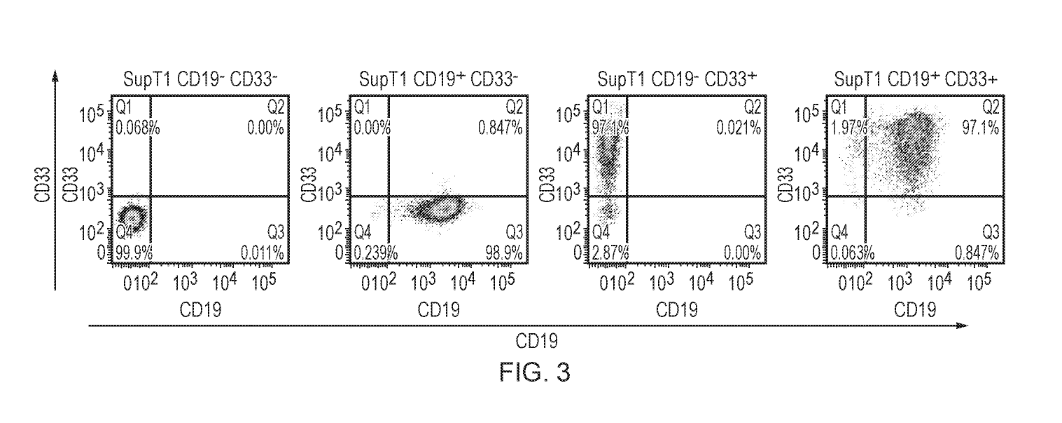

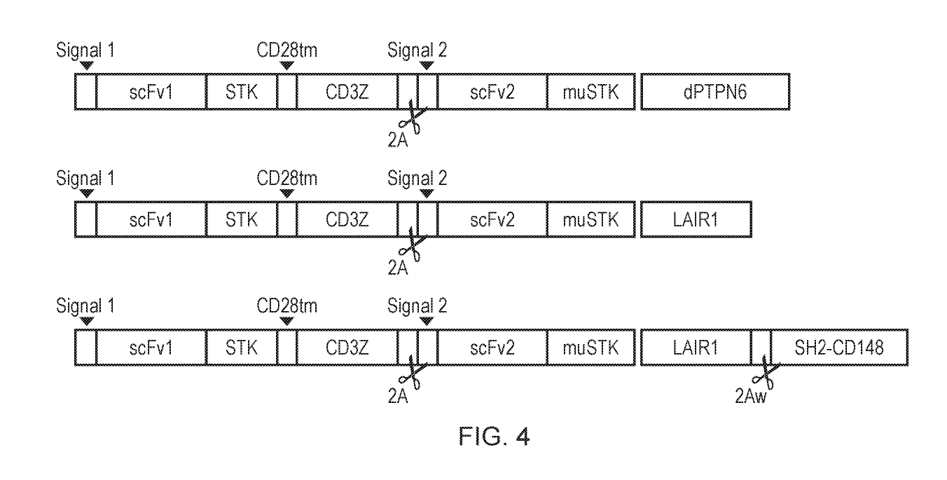

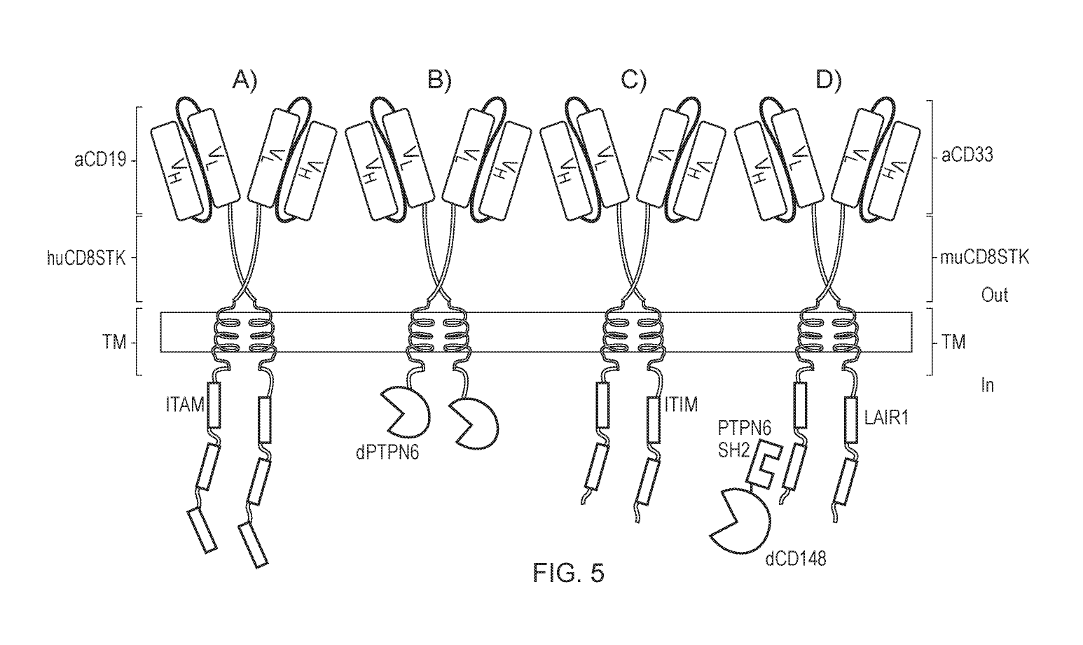

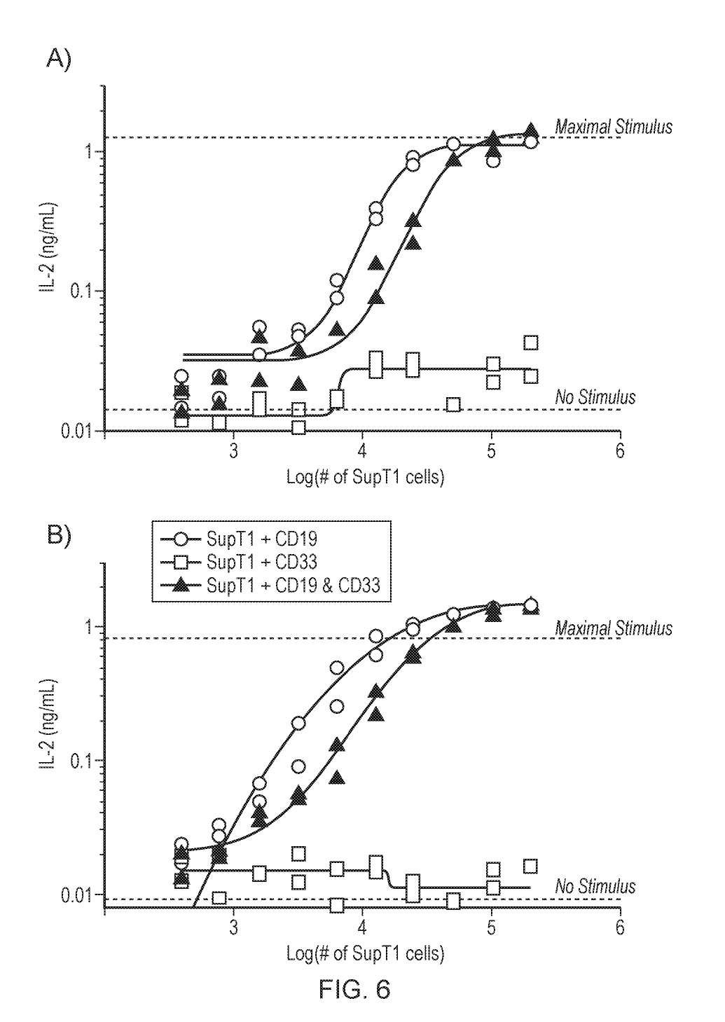

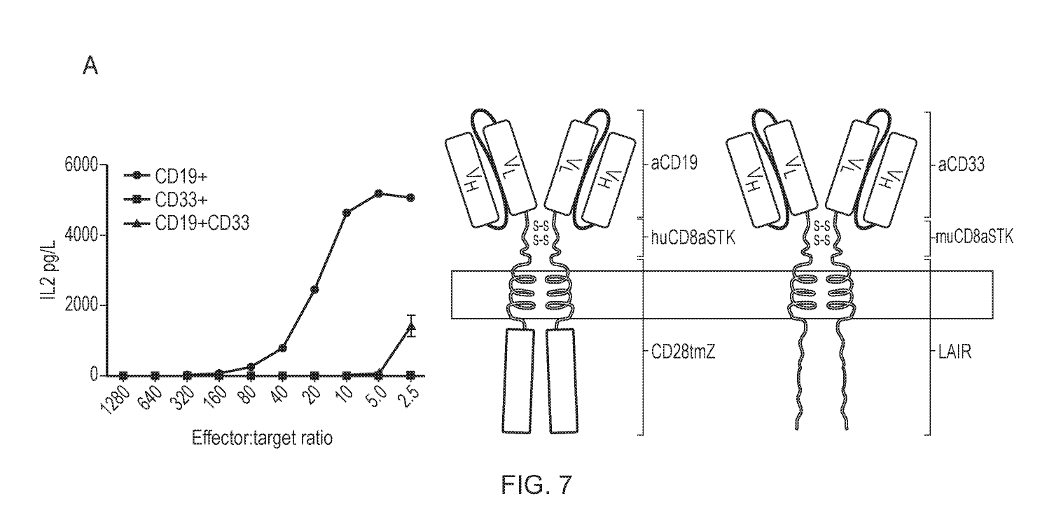

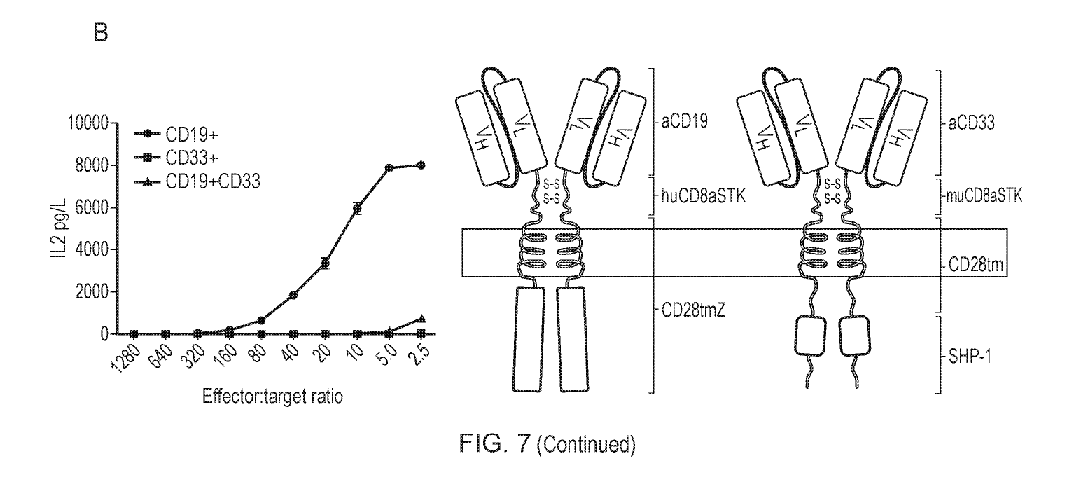

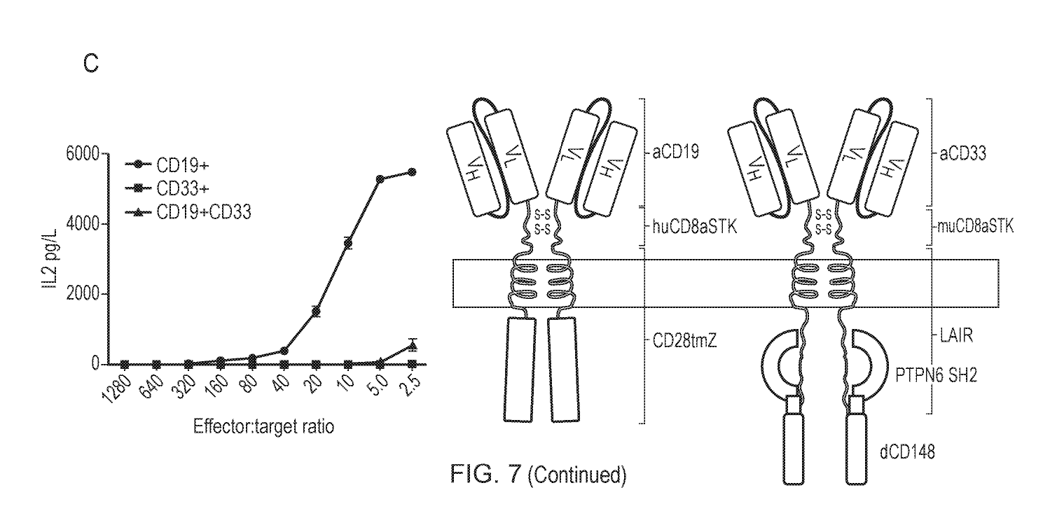

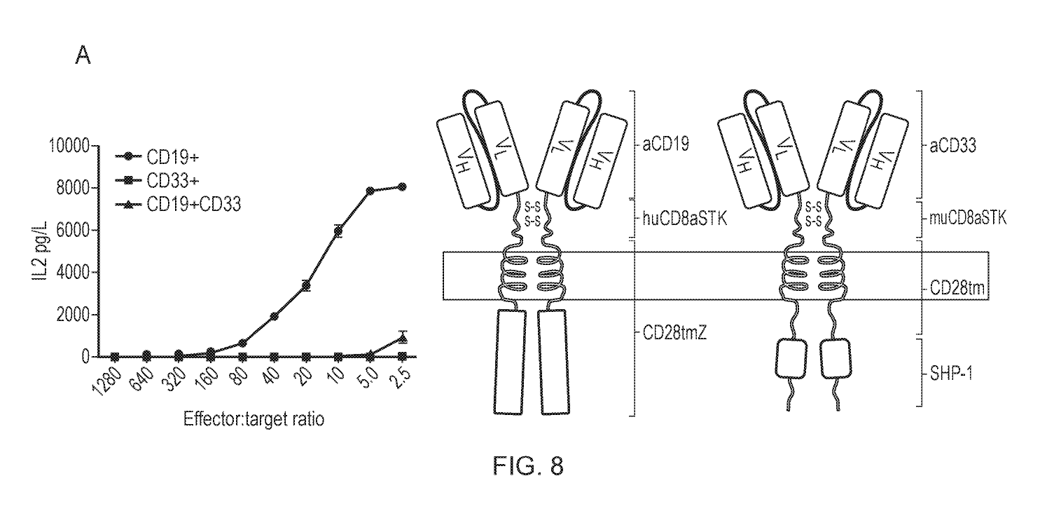

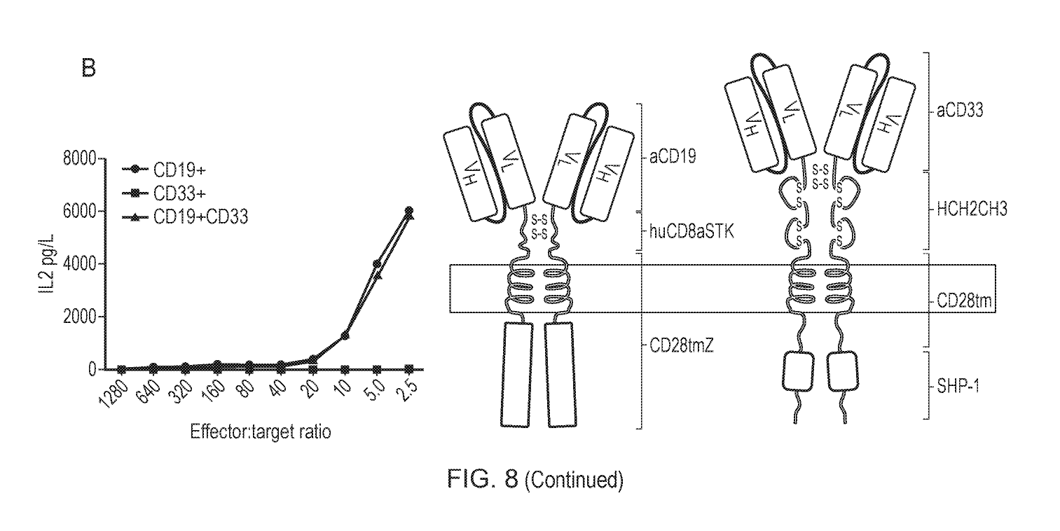

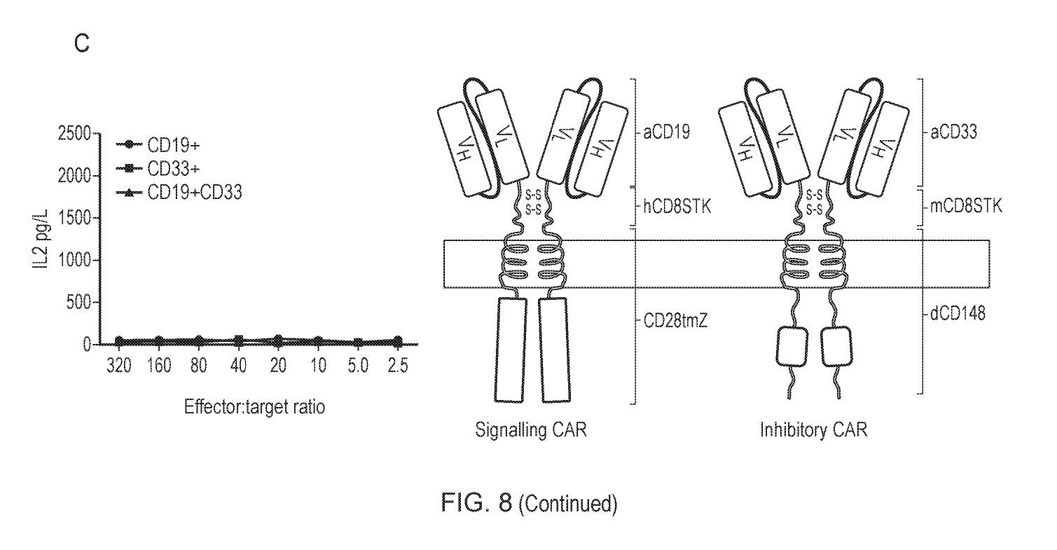

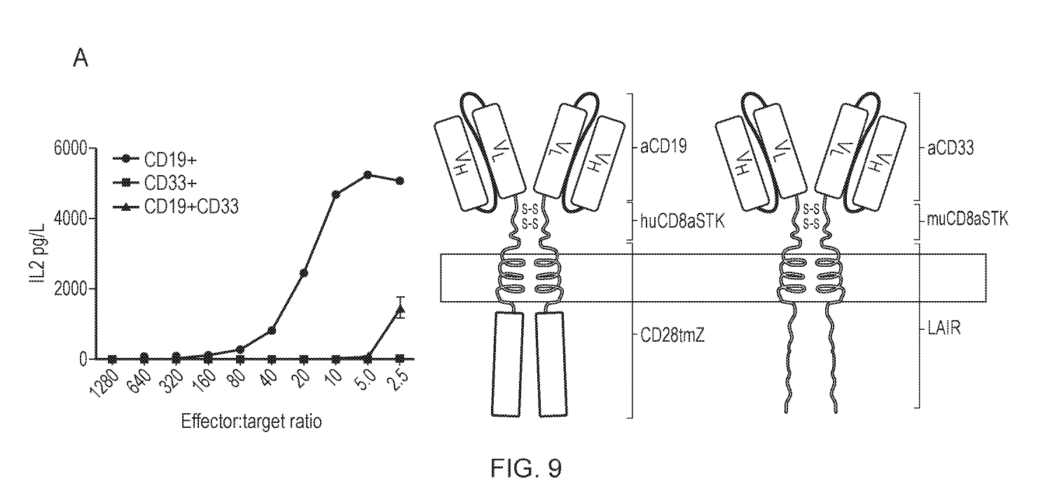

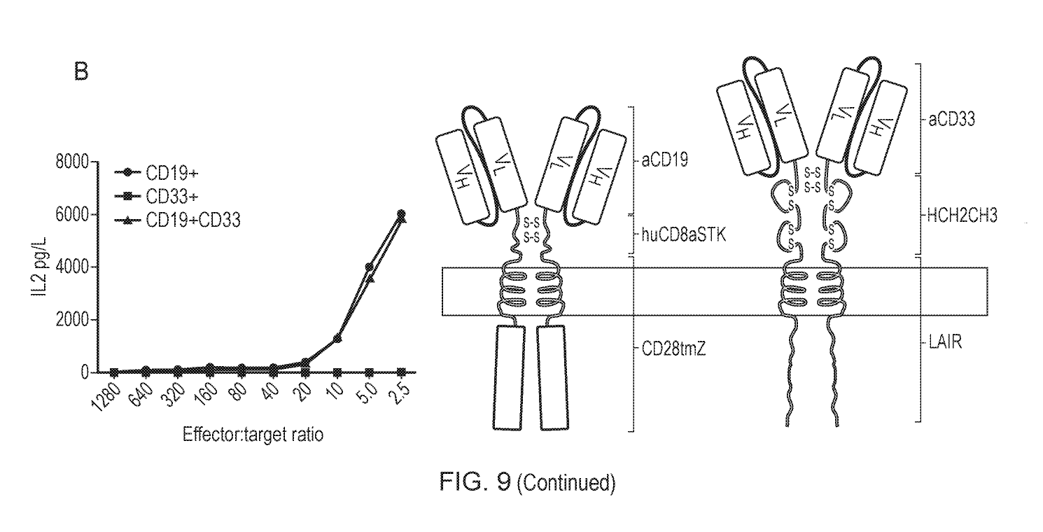

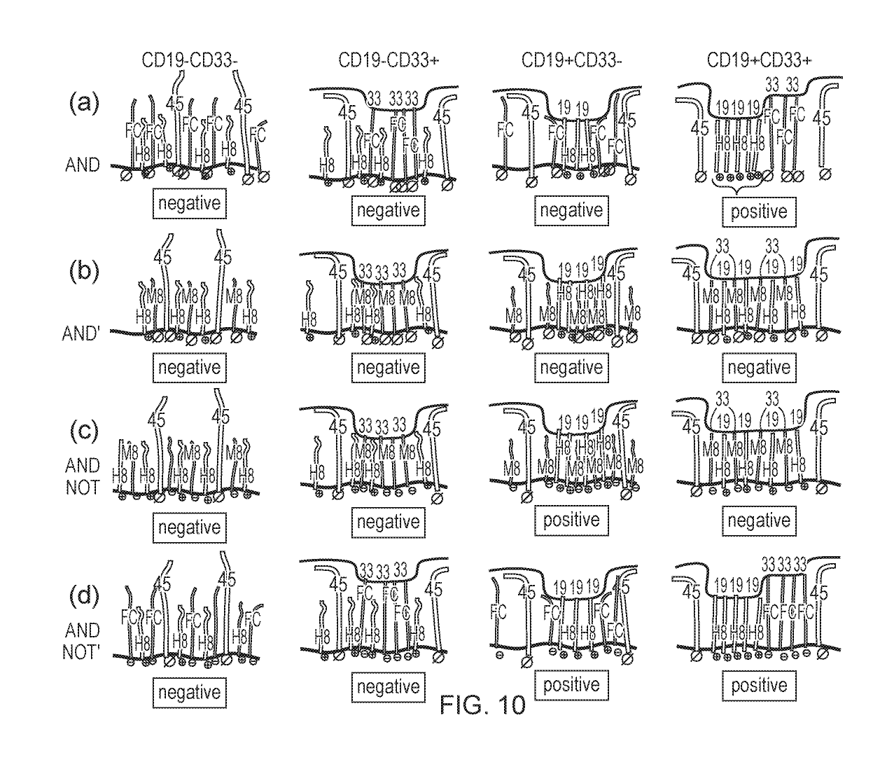

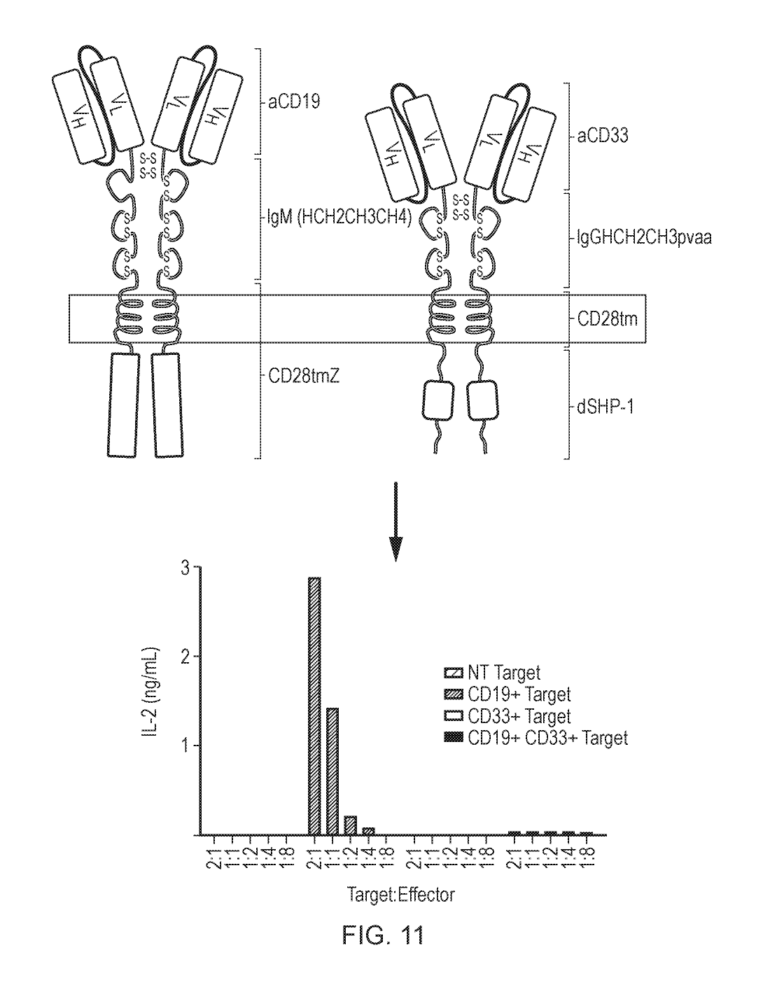

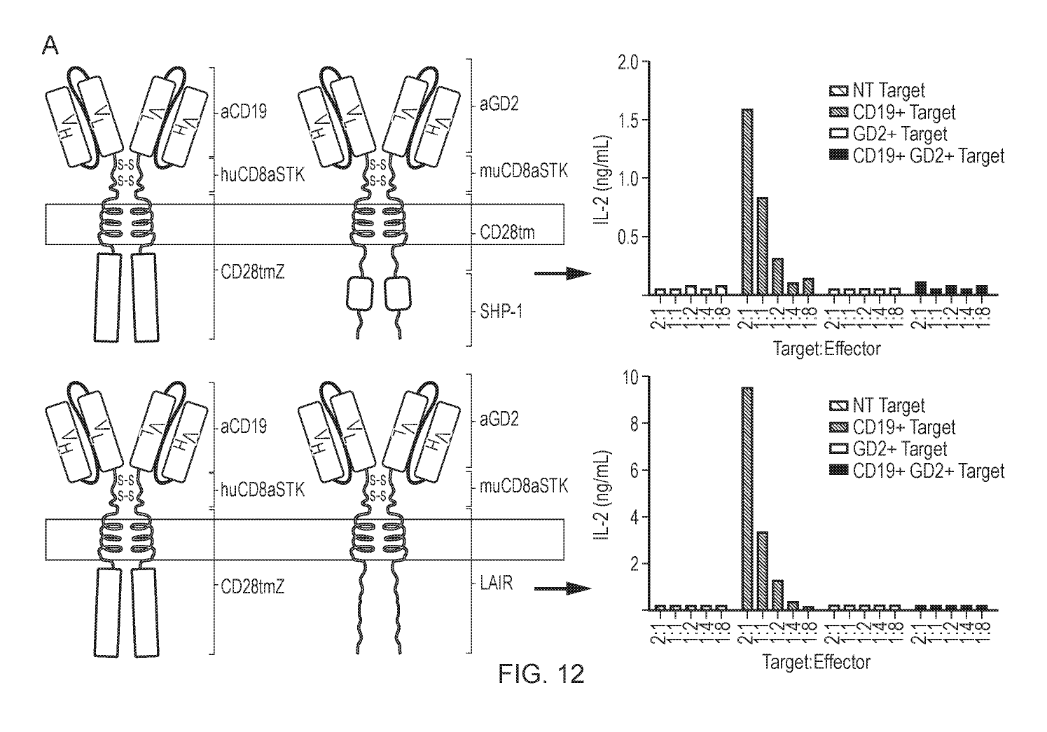

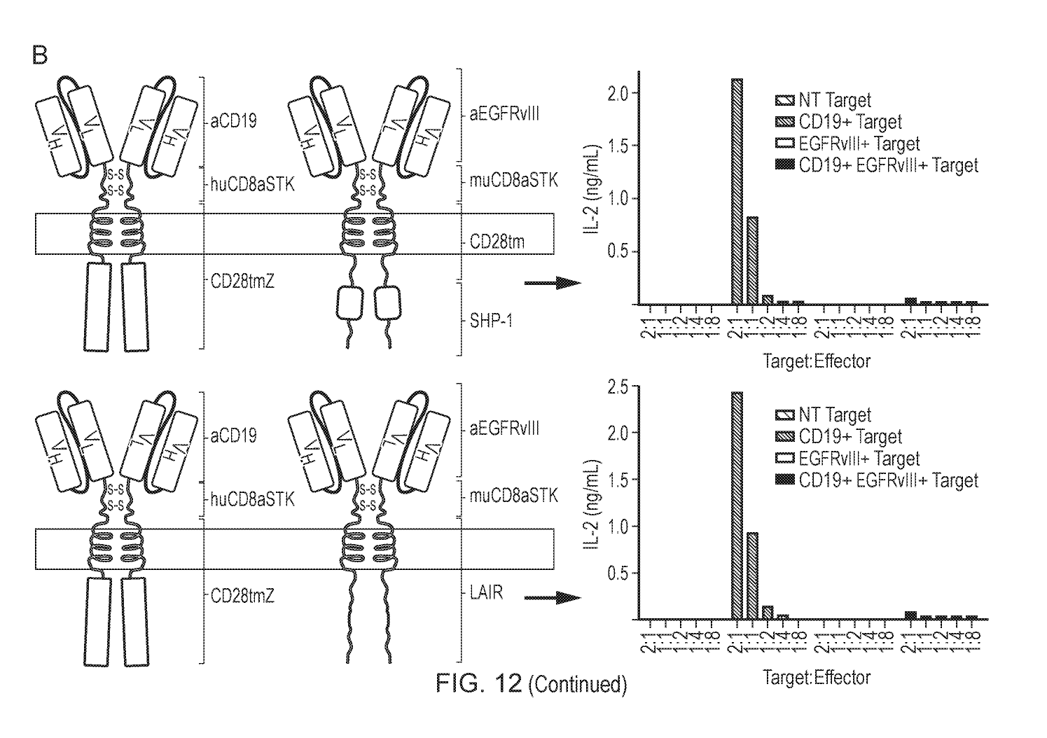

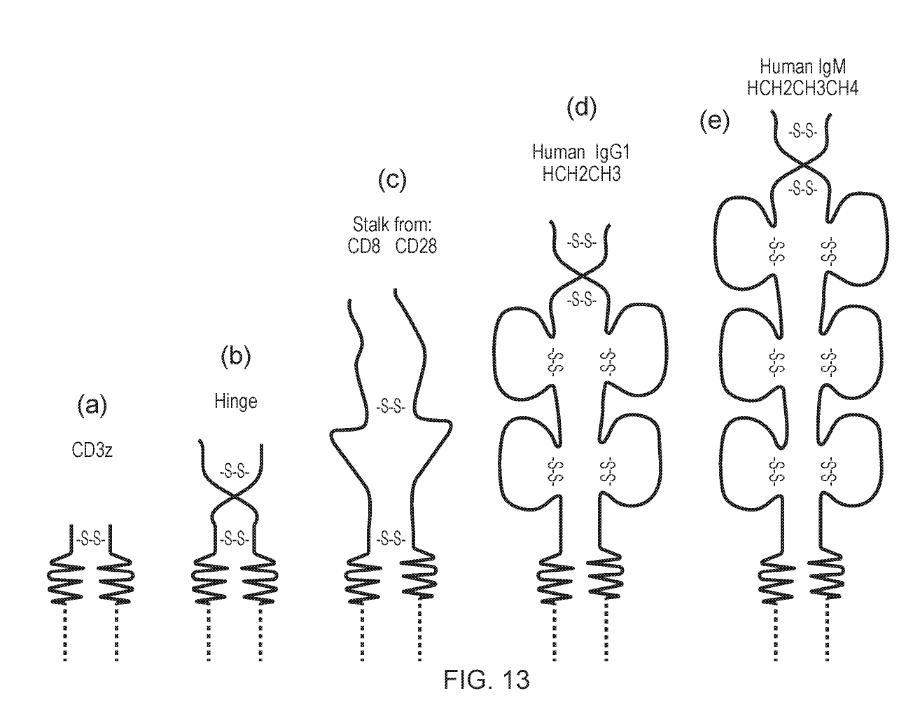

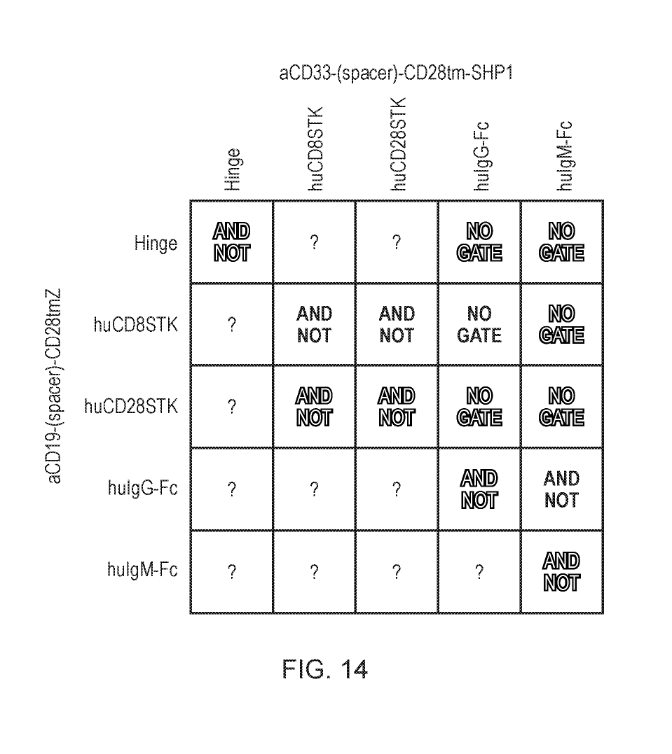

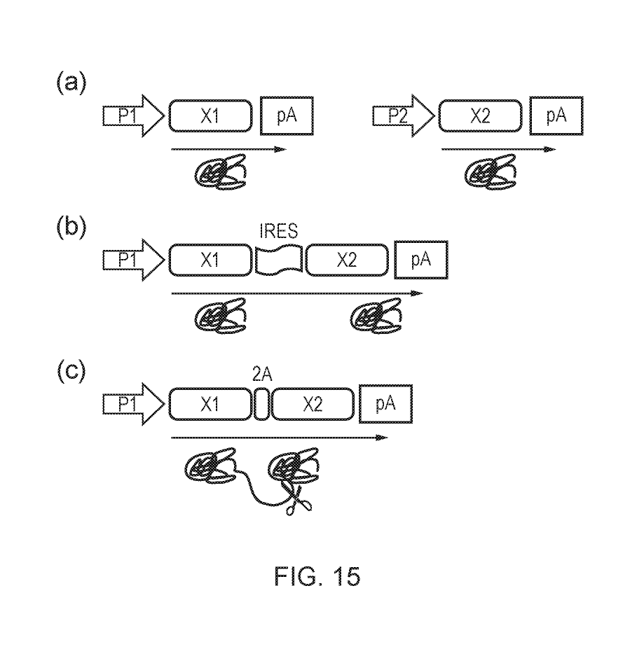

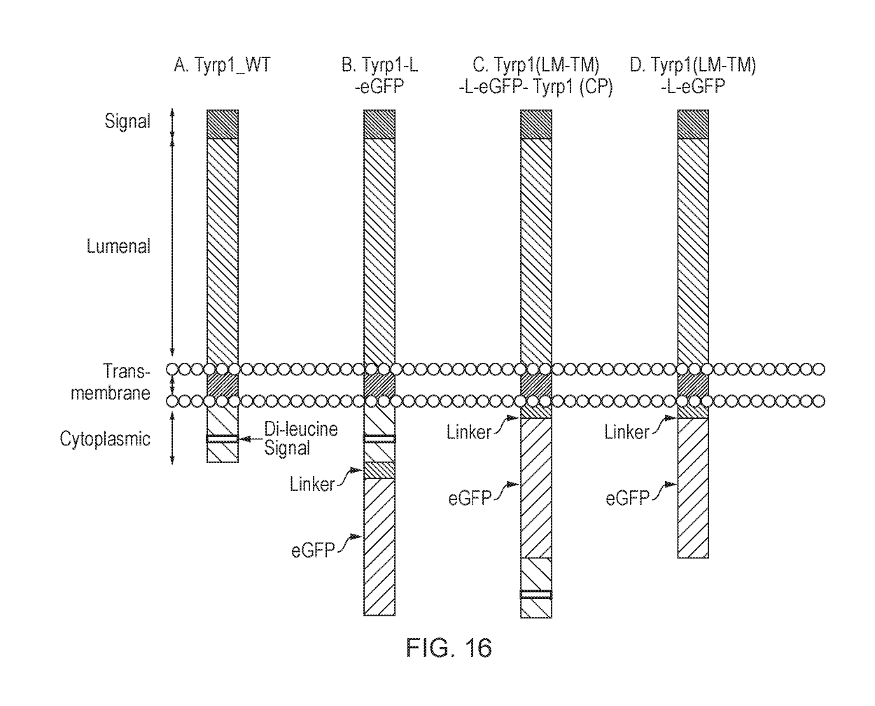

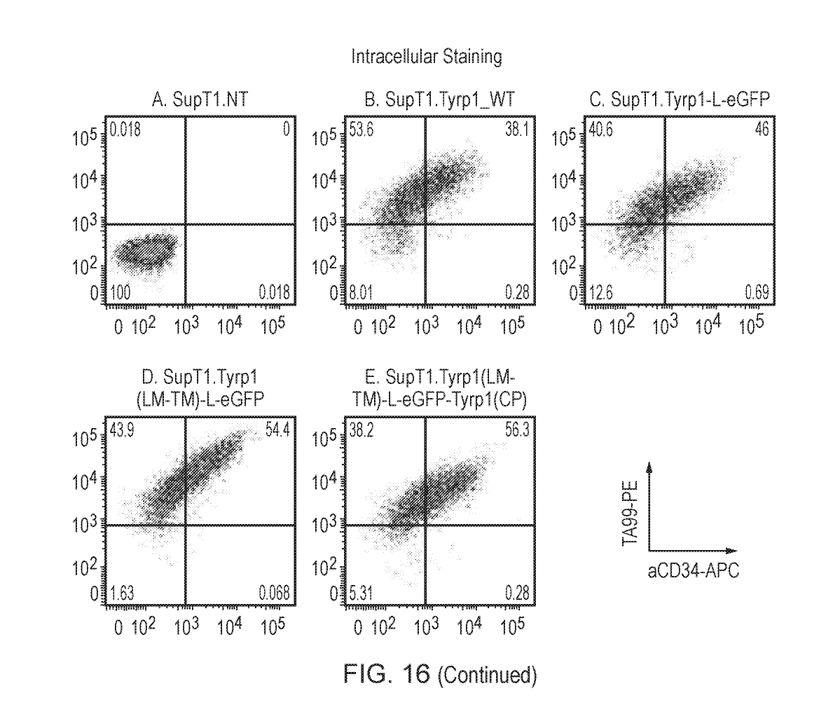

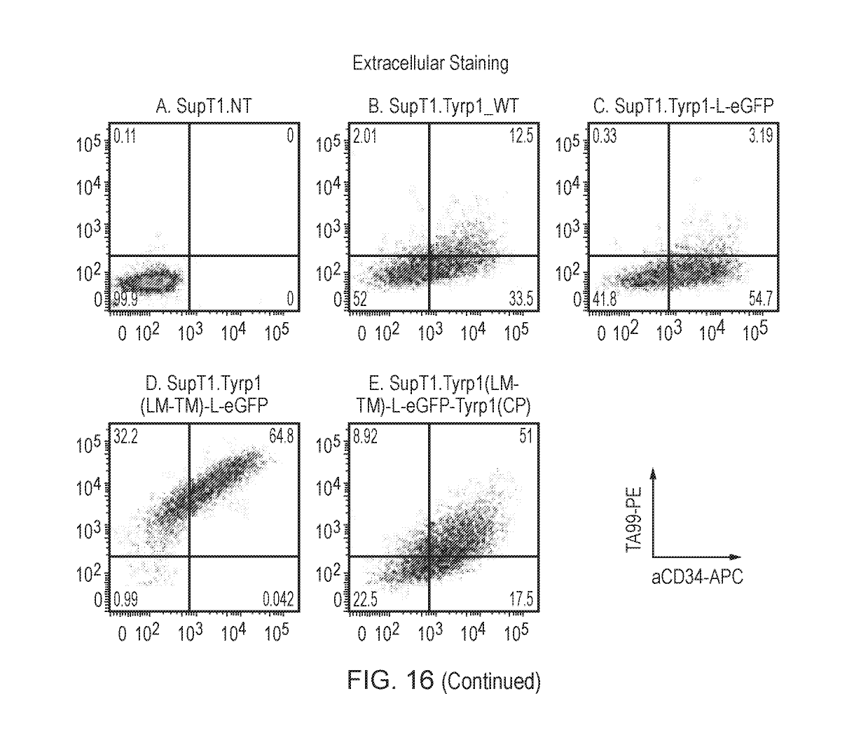

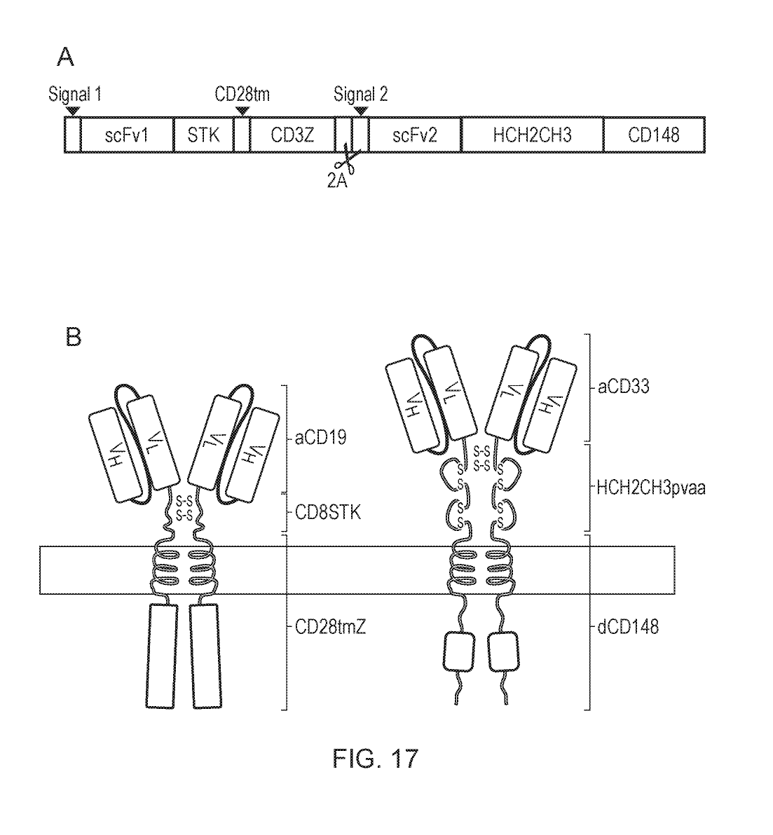

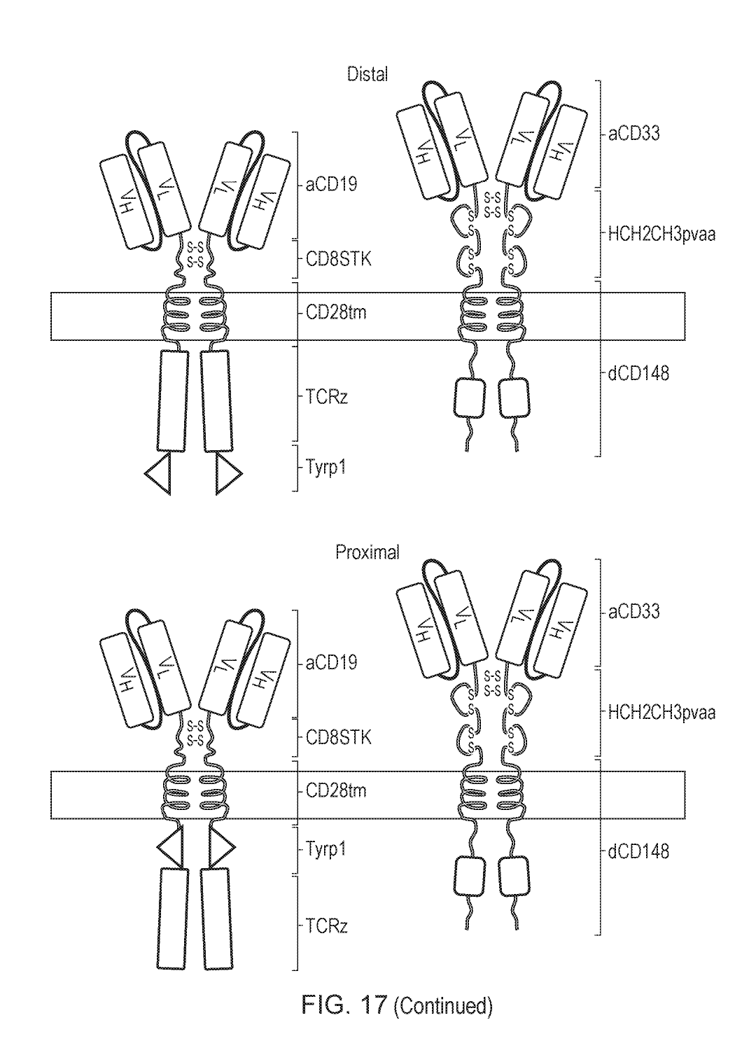

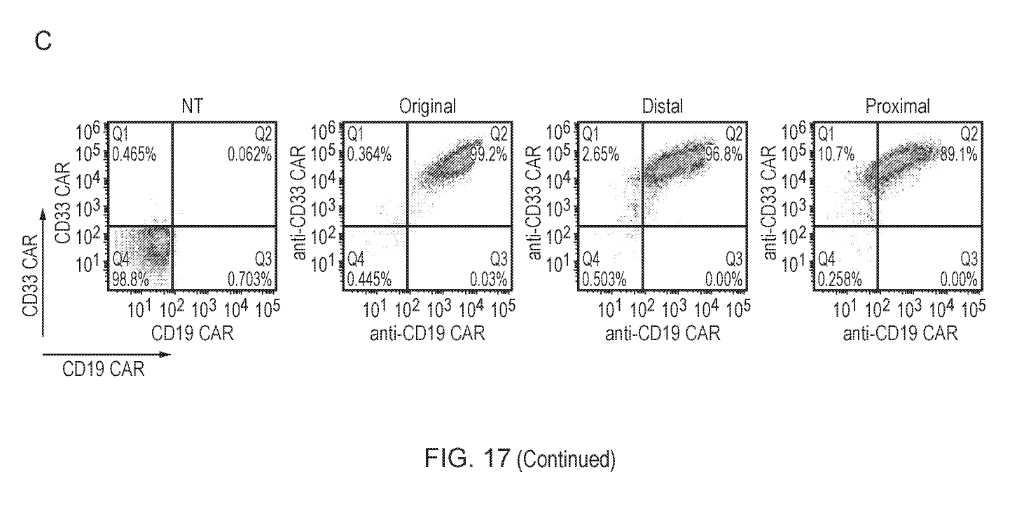

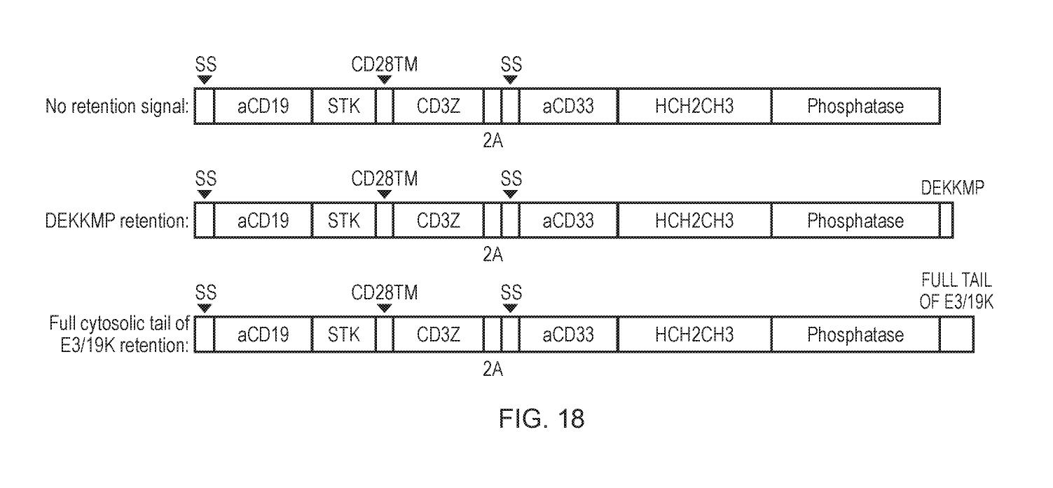

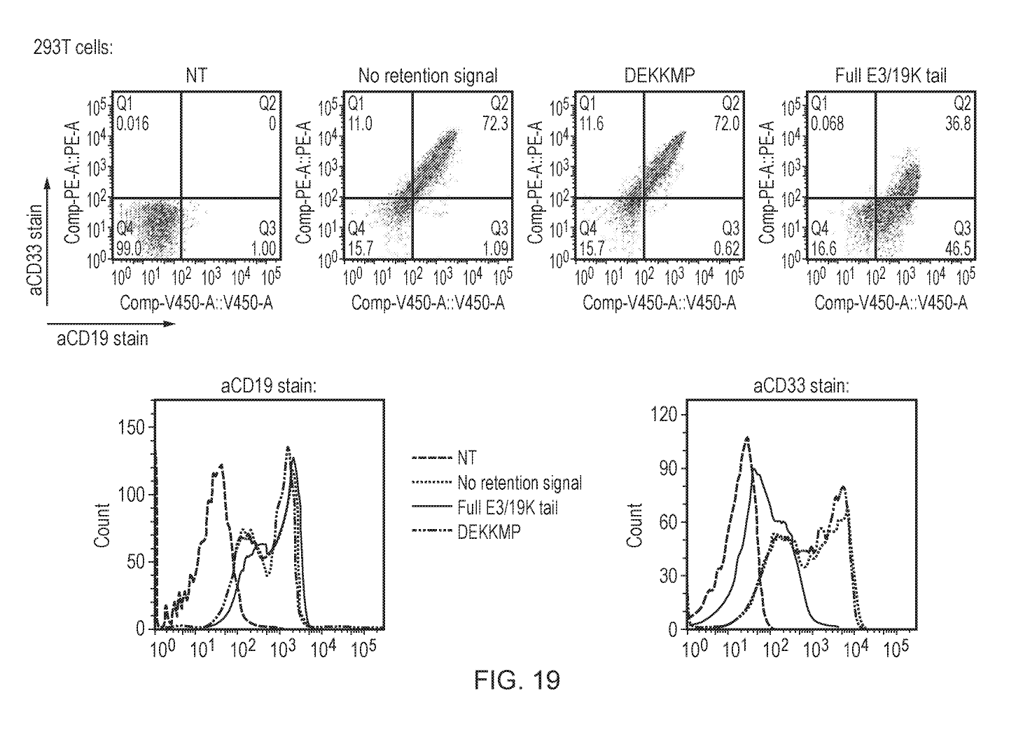

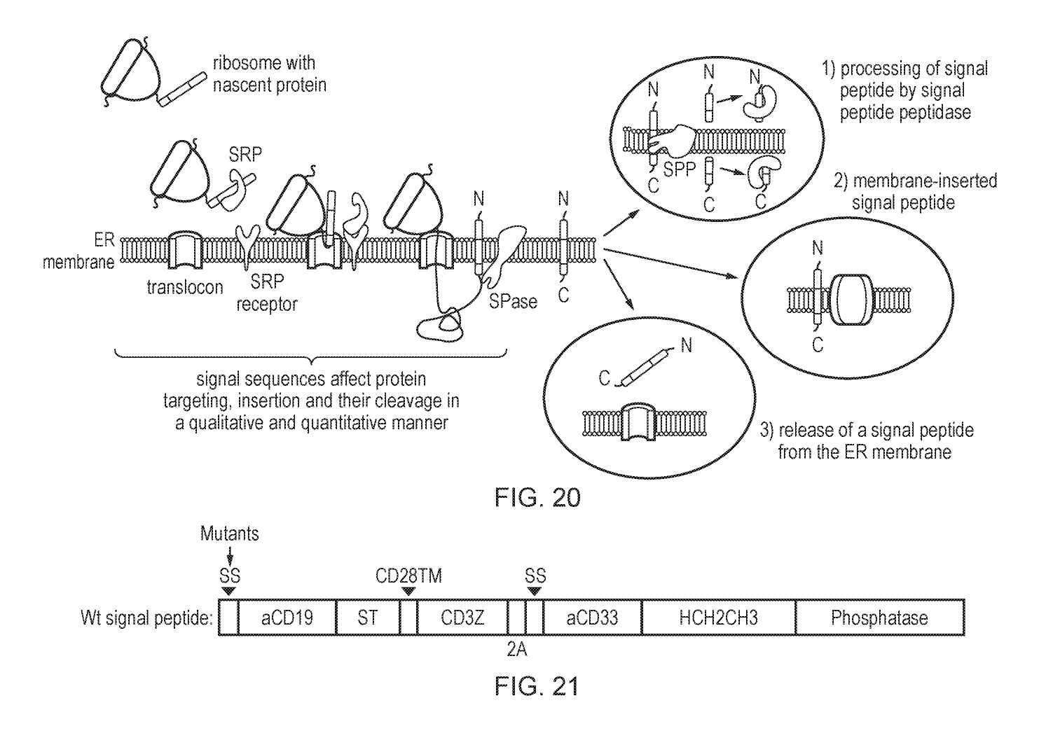

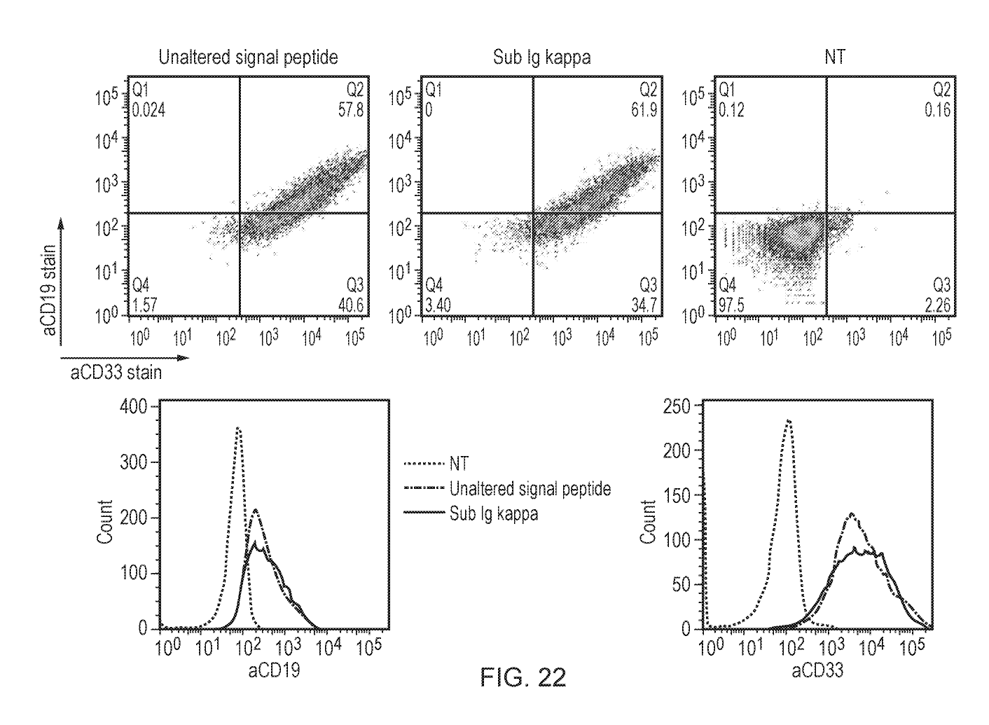

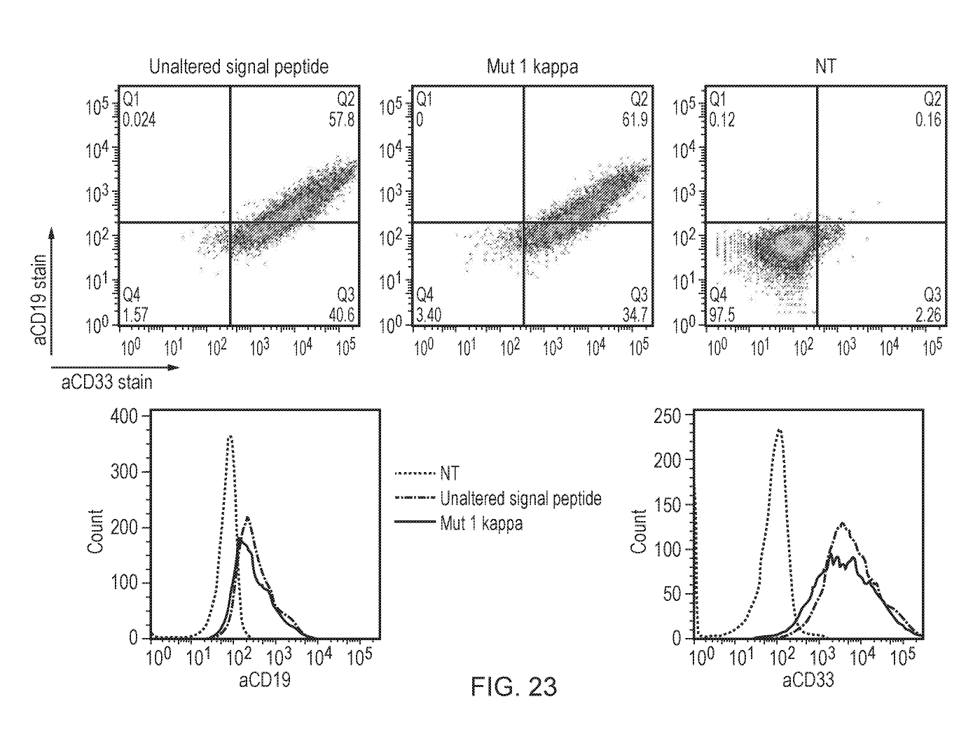

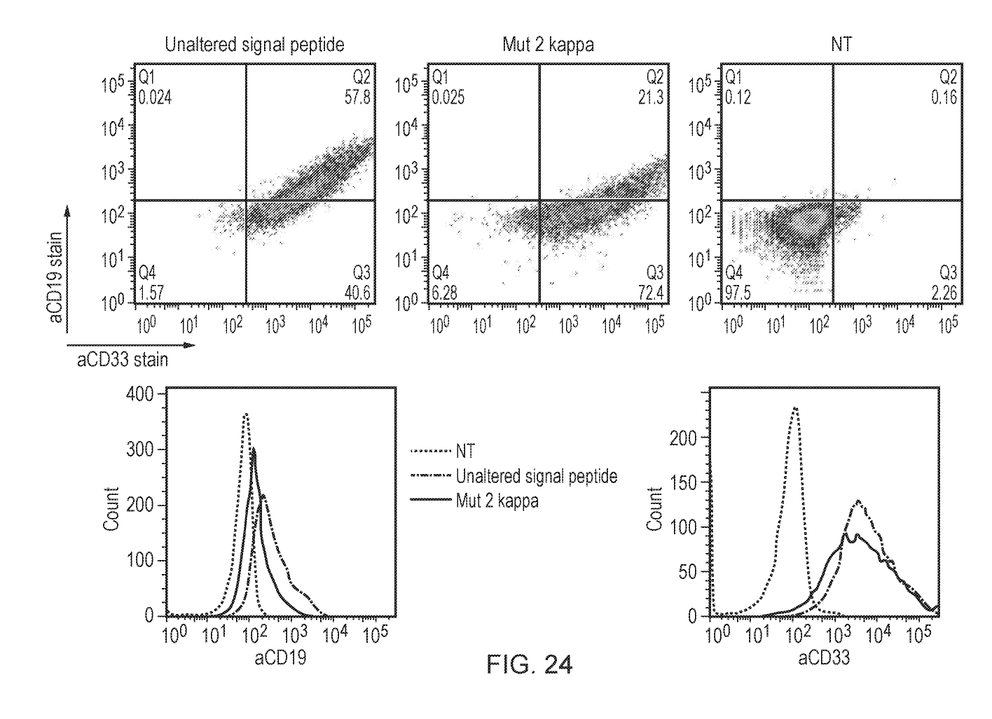

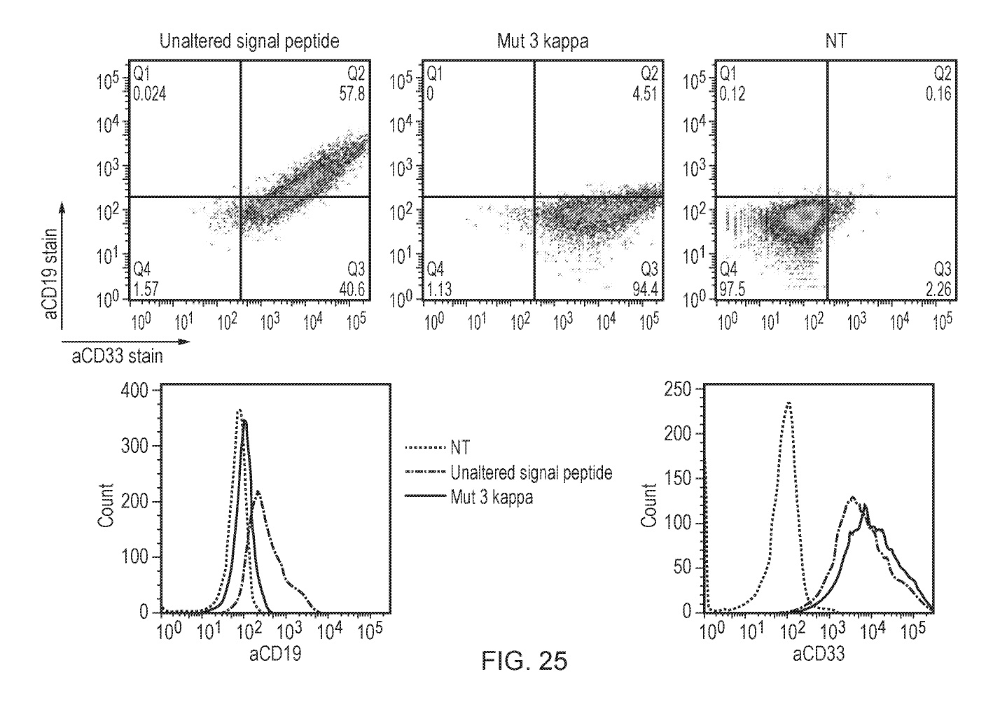

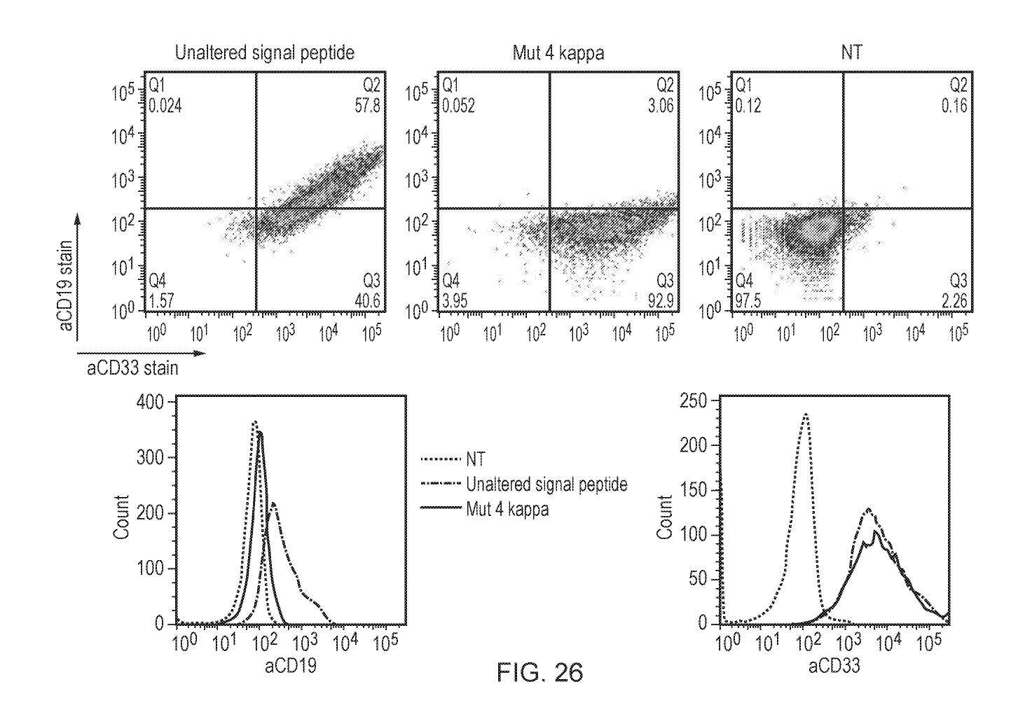

CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcϵR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3c results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmission of an activating signal to the T-cell it is expressed on. Thus the CAR directs the specificity and cytotoxicity of the T cell towards tumour cells expressing the targeted antigen.

The first aspect of the invention relates to a T-cell which co-expresses a first CAR and a second CAR such that a T-cell can recognize a desired pattern of expression on target cells in the manner of a logic gate as detailed in the truth tables: table 1, 2 and 3.

Both the first and second (and optionally subsequent) CARs comprise:

(i) an antigen-binding domain;
(ii) a spacer;
(iii) a transmembrane domain; and
(iii) an intracellular domain.

TABLE 1
Truth Table for CAR OR GATE

| Antigen A | Antigen B | Response |
|---|---|---|
| Absent | Absent | No activation |
| Absent | Present | Activation |
| Present | Absent | Activation |
| Present | Present | Activation |

TABLE 2
Truth Table for CAR AND GATE

| Antigen A | Antigen B | Response |
|---|---|---|
| Absent | Absent | No activation |
| Absent | Present | No Activation |
| Present | Absent | No Activation |
| Present | Present | Activation |

TABLE 3
Truth Table for CAR AND NOT GATE

| Antigen A | Antigen B | Response |
|---|---|---|
| Absent | Absent | No activation |
| Absent | Present | No Activation |
| Present | Absent | Activation |
| Present | Present | No Activation |

Using the nucleic acid construct of the present invention, the first and second CARs are produced as a polypeptide comprising both CARs, together with a cleavage site.

SEQ ID No. 4, 5 and 6 are examples of AND NOT gates, which comprise two CARs. The nucleic acid construct of the invention may comprise one or other part of the following amino acid sequences, which corresponds to a single CAR. One or both CAR sequences may be modified to include one or more intracellular retention signals, and/or to alter their signal peptides, as defined below.

SEQ ID No 4 Is a CAR AND NOT GATE which recognizes CD19 AND NOT CD33 based on PTPN6 phosphatase

SEQ ID No 5 Is an alternative implementation of the CAR AND NOT gate which recognizes CD19 AND NOT CD33 and is based on an ITIM containing endodomain from LAIR1

SEQ ID No 6. Is a further alternative implementation of the CAR AND NOT gate which recognizes CD19 AND NOT CD33 and recruits a PTPN6-CD148 fusion protein to an ITIM containing endodomain.

SEQ ID No. 4

MSLPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITKAGGGGSGGGGSGGGGSGGGGS
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSDPTTTPAPRPPTPAPTIASQPLSLRPEACR
PAAGGAVHTRGLDFACDIFWVLVVVGGVLACYSLLVTVAFIIFWVRRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPRRAEGRGSLLTCGDVEENPGPMAVPTQVLGLLLLWLTDARCDI
QMTQSPSSLSASVGDRVTITCRASEDIYFNLVVVYQQKPGKAPKLLIYDT
NRLADGVPSRFSGSGSGTQYTLTISSLQPEDFATYYCQHYKNYPLTFGQG
TKLEIKRSGGGGSGGGGSGGGGSGGGGSRSEVQLVESGGGLVQPGGSLRL
SCAASGFTLSNYGMHWIRQAPGKGLEWVSSISLNGGSTYYRDSVKGRFTI
SRDNAKSTLYLQMNSLRAEDTAVYYCAAQDAYTGGYFDYWGQGTLVTVSS
MDPATTTKPVLRTPSPVHPTGTSQPQRPEDCRPRGSVKGTGLDFACDIYW
APLAGICVALLLSLIITLICYHRSRKRVCKSGGGSFWEEFESLQKQEVKN
LHQRLEGQRPENKGKNRYKNILPFDHSRVILQGRDSNIPGSDYINANYIK
NQLLGPDENAKTYIASQGCLEATVNDFWQMAWQENSRVIVMTTREVEKGR
NKCVPYWPEVGMQRAYGPYSVTNCGEHDTTEYKLRTLQVSPLDNGDLIRE
IWHYQYLSWPDHGVPSEPGGVLSFLDQINQRQESLPHAGPIIVHCSAGIG
RTGTIIVIDMLMENISTKGLDCDIDIQKTIQMVRAQRSGMVQTEAQYKFI
YVAIAQFIETTKKKL

SEQ ID No. 4 breaks down as follows:

Signal peptide derived from Human CD8a:
MSLPVTALLLPLALLLHAARP
scFv aCD19:
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVS
Linker:
SD
Human CD8aSTK:
PTTTPAPRPPTPAPTIASQPLSLRPEACRPAAGGAVHTRGLDFACDI
Human CD28TM:
FWVLVVVGGVLACYSLLVTVAFIIFWV
Human CD3zeta intracellular domain:
RRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
2A peptide:
RAEGRGSLLTCGDVEENPGP
Signal peptide derived from mouse Ig kappa:
MAVPTQVLGLLLLWLTDA
scFv aCD33:
RCDIQMTQSPSSLSASVGDRVTITCRASEDIYFNLVWYQQKPGKAPKLLI
YDTNRLADGVPSRFSGSGSGTQYTLTISSLQPEDFATYYCQHYKNYPLTF
GQGTKLEIKRSGGGGSGGGGSGGGGSGGGGSRSEVQLVESGGGLVQPGGS
LRLSCAASGFTLSNYGMHWIRQAPGKGLEWVSSISLNGGSTYYRDSVKGR
FTISRDNAKSTLYLQMNSLRAEDTAVYYCAAQDAYTGGYFDYWGQGTLVT
VSSM
Linker:
DPA
Mouse CD8aSTK:
TTTKPVLRTPSPVHPTGTSQPQRPEDCRPRGSVKGTGLDFACD
Mouse CD8aTM:
IYWAPLAGICVALLLSLIITLI
Truncated Mouse CD8a intracellular domain:
CYHRSRKRVCK
Linker:
SGGGS
Truncated Human SHP-1:
FWEEFESLQKQEVKNLHQRLEGQRPENKGKNRYKNILPFDHSRVILQGRD
SNIPGSDYINANYIKNQLLGPDENAKTYIASQGCLEATVNDFWQMAWQEN
SRVIVMTTREVEKGRNKCVPYWPEVGMQRAYGPYSVTNCGEHDTTEYKLR
TLQVSPLDNGDLIREIWHYQYLSWPDHGVPSEPGGVLSFLDQINQRQESL
PHAGPIIVHCSAGIGRTGTIIVIDMLMENISTKGLDCDIDIQKTIQMVRA
QRSGMVQTEAQYKFIYVAIAQFIETTKKKL

SEQ ID No. 5

MSLPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITKAGGGGSGGGGSGGGGSGGGGS
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSDPTTTPAPRPPTPAPTIASQPLSLRPEACR
PAAGGAVHTRGLDFACDIFWVLVVVGGVLACYSLLVTVAFIIFWVRRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPRRAEGRGSLLTCGDVEENPGPMAVPTQVLGLLLLWLTDARCDI
QMTQSPSSLSASVGDRVTITCRASEDIYFNLVWYQQKPGKAPKLLIYDTN
RLADGVPSRFSGSGSGTQYTLTISSLQPEDFATYYCQHYKNYPLTFGQGT
KLEIKRSGGGGSGGGGSGGGGSGGGGSRSEVQLVESGGGLVQPGGSLRLS
CAASGFTLSNYGMHWIRQAPGKGLEWVSSISLNGGSTYYRDSVKGRFTIS
RDNAKSTLYLQMNSLRAEDTAVYYCAAQDAYTGGYFDYWGQGTLVTVSSM
DPATTTKPVLRTPSPVHPTGTSQPQRPEDCRPRGSVKGTGLDFACDILIG
VSVVFLFCLLLLVLFCLHRQNQIKQGPPRSKDEEQKPQQRPDLAVDVLER
TADKATVNGLPEKDRETDTSALAAGSSQEVTYAQLDHWALTQRTARAVSP
QSTKPMAESITYAAVARH

SEQ ID No. 5 breaks down as follows:

Signal peptide derived from Human CD8a:
MSLPVTALLLPLALLLHAARP
scFv aCD19:
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVS
Linker:
SD
Human CD8aSTK:
PTTTPAPRPPTPAPTIASQPLSLRPEACRPAAGGAVHTRGLDFACDI
Human CD28TM:
FWVLVVVGGVLACYSLLVTVAFIIFWV
Human CD3zeta intracellular domain:
RRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
2A peptide:
RAEGRGSLLTCGDVEENPGP
Signal peptide derived from mouse Ig kappa:
MAVPTQVLGLLLLWLTDA
scFv aCD33:
RCDIQMTQSPSSLSASVGDRVTITCRASEDIYFNLVWYQQKPGKAPKLLI
YDTNRLADGVPSRFSGSGSGTQYTLTISSLQPEDFATYYCQHYKNYPLTF
GQGTKLEIKRSGGGGSGGGGSGGGGSGGGGSRSEVQLVESGGGLVQPGGS
LRLSCAASGFTLSNYGMHWIRQAPGKGLEWVSSISLNGGSTYYRDSVKGR
FTISRDNAKSTLYLQMNSLRAEDTAVYYCAAQDAYTGGYFDYWGQGTLVT
VSSM
Linker:
DPA
Mouse CD8aSTK:
TTTKPVLRTPSPVHPTGTSQPQRPEDCRPRGSVKGTGLDFACD
Human LAIR-1 TM:
ILIGVSVVFLFCLLLLVLFCL
Human LAIR-1 intracellular domain:
HRQNQIKQGPPRSKDEEQKPQQRPDLAVDVLERTADKATVNGLPEKDRET
DTSALAAGSSQEVTYAQLDHWALTQRTARAVSPQSTKPMAESITYAAVAR
H

SEQ ID No. 6

MSLPVTALLLPLALLLHAARPDIQMTQTTSSLSASLGDRVTISCRASQDI
SKYLNVVYQQKPDGTVKLLIYHTSRLHSGVPSRFSGSGSGTDYSLTISNL
EQEDIATYFCQQGNTLPYTFGGGTKLEITKAGGGGSGGGGSGGGGSGGGG
SEVKLQESGPGLVAPSQSLSVTCTVSGVSLPDYGVSWIRQPPRKGLEWLG
VIWGSETTYYNSALKSRLTIIKDNSKSQVFLKMNSLQTDDTAIYYCAKHY
YYGGSYAMDYWGQGTSVTVSSDPTTTPAPRPPTPAPTIASQPLSLRPEAC
RPAAGGAVHTRGLDFACDIFVVVLVVVGGVLACYSLLVTVAFIIFWVRRV
KFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYD
ALHMQALPPRRAEGRGSLLTCGDVEENPGPMAVPTQVLGLLLLWLTDARC
DIQMTQSPSSLSASVGDRVTITCRASEDIYFNLVWYQQKPGKAPKLLIYD
TNRLADGVPSRFSGSGSGTQYTLTISSLQPEDFATYYCQHYKNYPLTFGQ
GTKLEIKRSGGGGSGGGGSGGGGSGGGGSRSEVQLVESGGGLVQPGGSLR
LSCAASGFTLSNYGMHWIRQAPGKGLEWVSSISLNGGSTYYRDSVKGRFT
ISRDNAKSTLYLQMNSLRAEDTAVYYCAAQDAYTGGYFDYWGQGTLVTVS
SMDPATTTKPVLRTPSPVHPTGTSQPQRPEDCRPRGSVKGTGLDFACDIL
IGVSVVFLFCLLLLVLFCLHRQNQIKQGPPRSKDEEQKPQQRPDLAVDVL
ERTADKATVNGLPEKDRETDTSALAAGSSQEVTYAQLDHWALTQRTARAV
SPQSTKPMAESITYAAVARHRAEGRGSLLTCGDVEENPGPWYHGHMSGGQ
AETLLQAKGEPWTFLVRESLSQPGDFVLSVLSDQPKAGPGSPLRVTHIKV
MCEGGRYTVGGLETFDSLTDLVEHFKKTGIEEASGAFVYLRQPYSGGGGS
FEAYFKKQQADSNCGFAEEYEDLKLVGISQPKYAAELAENRGKNRYNNVL
PYDISRVKLSVQTHSTDDYINANYMPGYHSKKDFIATQGPLPNTLKDFWR
MVWEKNVYAIIMLTKCVEQGRTKCEEYWPSKQAQDYGDITVAMTSEIVLP
EVVTIRDFTVKNIQTSESHPLRQFHFTSWPDHGVPDTTDLLINFRYLVRD
YMKQSPPESPILVHCSAGVGRTGTFIAIDRLIYQIENENTVDVYGIVYDL
RMHRPLMVQTEDQYVFLNQCVLDIVRSQKDSKVDLIYQNTTAMTIYENLA
PVTTFGKTNGYIASGS

SEQ ID No. 6 breaks down as follows:

Signal peptide derived from Human CD8a:
MSLPVTALLLPLALLLHAARP
scFv aCD19:
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVS
Linker:
SD
Human CD8aSTK:
PTTTPAPRPPTPAPTIASQPLSLRPEACRPAAGGAVHTRGLDFACDI
Human CD28TM:
FWVLVVVGGVLACYSLLVTVAFIIFWV
Human CD3zeta intracellular domain:
RRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
2A peptide:
RAEGRGSLLTCGDVEENPGP
Signal peptide derived from mouse Ig kappa:
MAVPTQVLGLLLLWLTDA
scFv aCD33:
RCDIQMTQSPSSLSASVGDRVTITCRASEDIYFNLVWYQQKPGKAPKLLI
YDTNRLADGVPSRFSGSGSGTQYTLTISSLQPEDFATYYCQHYKNYPLTF
GQGTKLEIKRSGGGGSGGGGSGGGGSGGGGSRSEVQLVESGGGLVQPGGS
LRLSCAASGFTLSNYGMHWIRQAPGKGLEWVSSISLNGGSTYYRDSVKGR
FTISRDNAKSTLYLQMNSLRAEDTAVYYCAAQDAYTGGYFDYWGQGTLVT
VSSM
Linker:
DPA
Mouse CD8aSTK:
TTTKPVLRTPSPVHPTGTSQPQRPEDCRPRGSVKGTGLDFACD
Human LAIR-1 TM:
ILIGVSVVFLFCLLLLVLFCL
Human LAIR-1 intracellular domain:
HRQNQIKQGPPRSKDEEQKPQQRPDLAVDVLERTADKATVNGLPEKDRET
DTSALAAGSSQEVTYAQLDHWALTQRTARAVSPQSTKPMAESITYAAVAR
H
2A peptide:
RAEGRGSLLTCGDVEENPGP
C-terminal SH2 domain derived from SHP-1:
WYHGHMSGGQAETLLQAKGEPWTFLVRESLSQPGDFVLSVLSDQPKAGPG
SPLRVTHIKVMCEGGRYTVGGLETFDSLTDLVEHFKKTGIEEASGAFVYL
RQPY
Linker:
SGGGGS
Truncated human CD148 intracellular domain:
FEAYFKKQQADSNCGFAEEYEDLKLVGISQPKYAAELAENRGKNRYNNVL
PYDISRVKLSVQTHSTDDYINANYMPGYHSKKDFIATQGPLPNTLKDFWR
MVWEKNVYAIIMLTKCVEQGRTKCEEYWPSKQAQDYGDITVAMTSEIVLP
EWTIRDFTVKNIQTSESHPLRQFHFTSWPDHGVPDTTDLLINFRYLVRDY
MKQSPPESPILVHCSAGVGRTGTFIAIDRLIYQIENENTVDVYGIVYDLR
MHRPLMVQTEDQYVFLNQCVLDIVRSQKDSKVDLIYQNTTAMTIYENLAP
VTTFGKTNGYIASGS

The present invention relates to the modulation of the relative expression of the two or more CARs in an AND NOT gate. This may be done, for example, by including an intracellular retention signal in one or other CAR.

In relation to SEQ ID NO. 4, 5 and 6 given above, a suitable position for the intracellular retention signal may be readily determined based on the position of the retention signal, or signals of a similar type, in its native protein. The modulatory effect of the retention signal may also be fine tuned by choosing a certain position for the retention signal in the molecule.

For example, the nucleic acid construct may comprise a tyrp-1 retention protein sequence, such as:

(SEQ ID No. 7)

RARRSMDEANQPLLTDQYQCYAEEYEKLQNPNQSVV

Such a sequence may be included, for example, in the CD19 CAR sequence in order for the relative expression level of CD19 CAR to be reduced with respect to CD33 CAR.

The position of the tyrp-1 retention signal in the aCD19 receptor will alter the amount of reduction: for low expression levels, the tyrp-1 retention signal may be placed between “Human CD28TM” and “Human CD3zeta intracellular domain”; for medium expression levels the tyrp-1 retention signal may be placed between “Human CD3zeta intracellular domain” and “2A peptide” in SEQ ID NO. 4, 5 or 6.

Alternatively, the nucleic acid construct may comprise the Adenoviral E3/19K intracellular retention signal. The intracellular retention signal may comprise the E3/19K cytosolic domain KYKSRRSFIDEKKMP (SEQ ID No. 2) or a portion thereof, such as the sequence DEKKMP (SEQ ID No. 3).

The E3/19K retention signal may be positioned at the C-terminus of the CAR whose expression is to be reduced.

The nucleic acid construct of the invention may encode SEQ ID No. 4, 5 or 6, or any of their components parts, such as the CD19 CAR or the CD33 CAR.

The nucleic acid construct of the invention may encode a variant of the CAR-encoding part of the sequence shown as SEQ ID No. 4, 5 or 6 having at least 80, 85, 90, 95, 98 or 99% sequence identity, provided that the variant sequence is a CAR having the required properties.

Methods of sequence alignment are well known in the art and are accomplished using suitable alignment programs. The % sequence identity refers to the percentage of amino acid or nucleotide residues that are identical in the two sequences when they are optimally aligned. Nucleotide and protein sequence homology or identity may be determined using standard algorithms such as a BLAST program (Basic Local Alignment Search Tool at the National Center for Biotechnology Information) using default parameters, which is publicly available at http://blast.ncbi.nlm.nih.gov. Other algorithms for determining sequence identity or homology include: LALIGN (http://www.ebi.ac.uk/Tools/psa/lalign/ and http://www.ebi.ac.uk/Tools/psa/lalign/nucleotide.html), AMAS (Analysis of Multiply Aligned Sequences, at http://www.compbio.dundee.ac.uk/Software/Amas/amas.html), FASTA (http://www.ebi.ac.uk/Tools/msa/clustalo/), Clustal Omega (http://www.ebi.ac.uk/Tools/msa/clustalo/), SIM (http://web.expasy.org/sim/), and EMBOSS Needle (http://www.ebi.ac.uk/Tools/psa/emboss______needle/nucleotide.html).

The Car Logical and not Gate

In the AND NOT, one CAR comprises an activating endodomain and one CAR comprises an inhibitory endodomain such that this inhibitory CAR is only active when it recognizes its cognate antigen. Hence a T-cell engineered in this manner is activated in response to the sole presence of the first antigen but is not activated when both antigens are present. This invention is implemented by inhibitory CARs with a spacer that co-localise with the first CAR but either the phosphatase activity of the inhibitory CAR should not be so potent that it inhibits in solution, or the inhibitory endodomain in fact recruits a phosphatase solely when the inhibitory CAR recognizes its cognate target. Such endodomains are termed “ligation-on” or semi-inhibitory herein.

This invention is of use in refining targeting when a tumour can be distinguished from normal tissue by the presence of tumour associated antigen and the loss of an antigen expressed on normal tissue. The AND NOT gate is of considerable utility in the field of oncology as it allows targeting of an antigen which is expressed by a normal cell, which normal cell also expresses the antigen recognised by the CAR comprising the activating endodomain. An example of such an antigen is CD33 which is expressed by normal stem cells and acute myeloid leukaemia (AML) cells. CD34 is expressed on stem cells but not typically expressed on AML cells. A T-cell recognizing CD33 AND NOT CD34 would result in destruction of leukaemia cells but sparing of normal stem cells.

Potential antigen pairs for use with AND NOT gates are shown in Table 6.

TABLE 6

| | | Normal cell which | Antigen expressed by normal |
|---|---|---|---|
| Disease | TAA | expresses TAA | cell but not cancer cell |
| AML | CD33 | stem cells | CD34 |
| Myeloma | BCMA | Dendritic cells | CD1c |
| B-CLL | CD160 | Natural Killer cells | CD56 |
| Prostate | PSMA | Neural Tissue | NCAM |
| cancer | | | |
| Bowel | A33 | Normal bowel | HLA class I |
| cancer | | epithelium | |

Compound Gates

The present invention allows compound gates to be made e.g. a T-cell which triggers in response to patterns of more than two target antigens. For example, it is possible to make a gate which recognises the pattern of three antigens (A AND NOT B) AND C: Here CAR against antigen A has an activating endodomain and co-localises with CAR against antigen B which has a conditionally inhibiting endodomain. CAR against antigen C has a spacer who segregates differently from A or B and is inhibitory.

Signal Peptide

The polypeptides A and B (and optionally others, C, D etc) encoded by the nucleic acid construct of the invention each comprise may a signal sequence so that when the polypeptide is expressed inside a cell the nascent protein is directed to the endoplasmic reticulum (ER) (see FIG. 20).

The term “signal peptide” is synonymous with “signal sequence”.

A signal peptide is a short peptide, commonly 5-30 amino acids long, present at the N-terminus of the majority of newly synthesized proteins that are destined towards the secretory pathway. These proteins include those that reside either inside certain organelles (for example, the endoplasmic reticulum, golgi or endosomes), are secreted from the cell, and transmembrane proteins.

Signal peptides commonly contain a core sequence which is a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide is commonly positioned at the amino terminus of the molecule, although some carboxy-terminal signal peptides are known.

As mentioned above, signal sequences have a tripartite structure, consisting of a hydrophobic core region (h-region) flanked by an n- and c-region. The latter contains the signal peptidase (SPase) consensus cleavage site. Usually, signal sequences are cleaved off co-translationally, the resulting cleaved signal sequences are termed signal peptides.

In the signal peptide from the murine Ig kappa chain V-III region, which has the sequence: METDTLILWVLLLLVPGSTG: the n-region has the sequence METD; the h-region (shown in bold) has the sequence TLILVVVLLLLV; and the c-region has the sequence PGSTG.

In the nucleic acid construct of the present invention, the signal peptides of the two CARs may differ in the number of hydrophobic amino acids, to modulate the relative levels of expression of the CARs at the cell surface.

In the nucleic acid construct of the present invention the signal sequence of the two (or more) polypeptides therefore may differ in their h-regions. One polypeptide (which has higher relative expression) may have a greater number of hydrophobic amino acids in the h-region that the other polypeptide (which has lower relative expression). The signal peptide of the polypeptide with lower relative expression may comprise one or more amino acid mutations, such as substitutions or deletions, of hydrophobic amino acids in the h-region than the signal peptide of the polypeptide with lower relative expression.

The first signal peptide and the second signal peptide may have substantially the same n- and c-regions, but differ in the h-region as explained above. “Substantially the same” indicates that the n- and c-regions may be identical between the first and second signal peptide or may differ by one, two or three amino acids in the n- or c-chain, without affecting the function of the signal peptide.

The hydrophobic amino acids in the core may, for example be: Alanine (A); Valine (V); Isoleucine (I); Leucine (L); Methionine (M); Phenylalanine (P); Tyrosine (Y); or Tryptophan (W).

The hydrophobic acids mutated in order to alter signal peptide efficiency may be any from the above list, in particular: Valine (V); Isoleucine (I); Leucine (L); and Tryptophan (W).

Of the residues in the h-region, one signal peptide (for example, the altered signal peptide) may comprise at least 10%, 20%, 30%, 40% or 50% fewer hydrophobic amino acids than the other signal peptide (for example, the unaltered signal peptide).

Where the h-region comprises 5-15 amino acids, one signal peptide may comprise 1, 2, 3, 4 or 5 more hydrophobic amino acids than the other signal peptide.

The altered signal peptide may comprise 1, 2, 3, 4 or 5 amino acid deletions or substitutions of hydrophobic amino acids. Hydrophobic amino acids may be replaced with non-hydrophobic amino acids, such as hydrophilic or neutral amino acids.

Signal sequences can be detected or predicted using software techniques (see for example, http://www.predisi.de/).

A very large number of signal sequences are known, and are available in databases. For example, http://www.signalpeptide.de lists 2109 confirmed mammalian signal peptides in its database.

Table 5 provides a list of signal sequences purely for illustrative purposes. The hydrophobic core is highlighted in bold. This includes examples of amino acids which may be substituted or removed for the purposes of the present invention.

TABLE 5

| Accession | | | | |
|---|---|---|---|---|
| Number | Entry Name | Protein Name | Length | Signal Sequence (hydrophobic core) |
| P01730 | CD4_HUMAN | T-cell surface glycoprotein CD4 | 25 | MNRGVPFRHLLLVLQLALLPAATQG |
| P08575 | CD45_HUMAN | Leukocyte common antigen | 23 | MYLWLKLLAFGFAFLDTEVFVTG |
| P01732 | CD8A_HUMAN | T-cell surface glycoprotein CD8 | 21 | MALPVTALLLPLALLLHAARP |
| | | alpha chain | | |
| P10966 | CD8B_HUMAN | T-cell surface glycoprotein CD8 | 21 | MRPRLWLLLAAQLTVLHGNSV |
| | | beta chain | | |
| P06729 | CD2_HUMAN | T-cell surface antigen CD2 | 24 | MSFPCKFVASFLLIFNVSSKGAVS |
| P06127 | CD5_HUMAN | T-cell surface glycoprotein CD5 | 24 | MPMGSLQPLATLYLLGMLVASCLG |
| P09564 | CD7_HUMAN | T-cell antigen CD7 | 25 | MAGPPRLLLLPLLLALARGLPGALA |
| P17643 | TYRP1_HUMAN | 5,6-dihydroxyindole-2-carboxylic | 24 | MSAPKLLSLGCIFFPLLLFQQARA |
| | | acid oxidase | | |
| P00709 | LALBA_HUMAN | Alpha-lactalbumin | 19 | MRFFVPLFLVGILFPAILA |
| P16278 | BGAL_HUMAN | Beta-galactosidase | 23 | MPGFLVRILPLLLVLLLLGPTRG |
| P31358 | CD52_HUMAN | CAMPATH-1 antigen | 24 | MKRFLFLLLTISLLVMVQIQTGLS |
| Q6YHK3 | CD109_HUMAN | CD109 antigen | 21 | MQGPPLLTAAHLLCVCTAALA |
| P01024 | CO3_HUMAN | Complement C3 | 22 | MGPTSGPSLLLLLLTHLPLALG |
| P10144 | GRAB_HUMAN | Granzyme B | 18 | MQPILLLLAFLLLPRADA |
| P04434 | KV310_HUMAN | Ig kappa chain V-III region VH | 20 | MEAPAQLLFLLLLWLPDTTR |
| P06312 | KV401_HUMAN | Ig kappa chain V-IV region | 20 | MVLQTQVFISLLLWISGAYG |
| P06319 | LV605_HUMAN | Ig lambda chain V-VI region EB4 | 19 | MAWAPLLLTLLAHCTDCWA |
| P31785 | IL2RG_HUMAN | Cytokine receptor common gamma | 22 | MLKPSLPFTSLLFLQLPLLGVG |
| | | chain | | |
| Q8N4F0 | BPIL1_HUMAN | Bactericidal/permeability- | 20 | MAWASRLGLLLALLLPVVGA |
| | | increasing protein-like 1 | | |
| P55899 | FCGRN_HUMAN | IgG receptor FcRn large subunit | 23 | MGVPRPQPWALGLLLFLLPGSLG |
| | | p51 | | |

The mutated signal peptide comprises one or more mutation(s) such that it has fewer hydrophobic amino acids than the wild-type signal peptide from which it is derived. The term “wild type” means the sequence of the signal peptide which occurs in the natural protein from which it is derived. For example, the signal peptide described in the examples is the signal peptide from the murine Ig kappa chain V-III region, which has the wild-type sequence: METDTLILWVLLLLVPGSTG.

The term “wild-type” also includes signal peptides derived from a naturally occurring protein which comprise one or more amino acid mutations in the n- or c-region. For example it is common to modify a natural signal peptide with a conserved amino acid substitution on the N-terminus to introduce a restriction site. Such modified signal peptide sequences (which do not comprise any mutations in the h-region) are considered “wild-type” for the purposes of the present invention.

The present invention also relates to synthetic signal peptide sequences, which cannot be defined with reference to a wild-type sequence. In this embodiment, the signal peptide of the one polypeptide comprises fewer hydrophobic amino acids than the signal sequence of the other polypeptide. The two signal sequences may be derived from the same synthetic signal peptide sequence, but differ in the number of hydrophobic amino acids in the core region.

Antigen Binding Domain

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell.

The antigen binding domain may be based on a natural ligand of the antigen. For example, the antigen binding domain may comprise APRIL, the natural ligand of BCMA.

The antigen binding domain may comprise an affinity peptide from a combinatorial library or a de novo designed affinity protein/peptide.

Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

The spacer sequence for a CAR may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Examples of amino acid sequences for these spacers are given below:

(hinge-CH2CH3 of human IgG1)

SEQ ID No. 8

AEPKSPDKTHTCPPCPAPPVAGPSVFLFPPKPKDTLMIARTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGKKD
(human CD8 stalk):

SEQ ID No. 9

TTTPAPRPPTPAPTIASQPLSLRPEACRPAAGGAVHTRGLDFACDI
(human IgG1 hinge):

SEQ ID No. 10

AEPKSPDKTHTCPPCPKDPK
(CD2 ectodomain)

SEQ ID No. 11

KEITNALETWGALGQDINLDIPSFQMSDDIDDIKWEKTSDKKKIAQFRKE
KETFKEKDTYKLFKNGTLKIKHLKTDDQDIYKVSIYDTKGKNVLEKIFDL
KIQERVSKPKISWTCINTTLTCEVMNGTDPELNLYQDGKHLKLSQRVITH
KWTTSLSAKFKCTAGNKVSKESSVEPVSCPEKGLD
(CD34 ectodomain)

SEQ ID no. 12

SLDNNGTATPELPTQGTFSNVSTNVSYQETTTPSTLGSTSLHPVSQHGNE
ATTNITETTVKFTSTSVITSVYGNTNSSVQSQTSVISTVFTTPANVSTPE
TTLKPSLSPGNVSDLSTTSTSLATSPTKPYTSSSPILSDIKAEIKCSGIR
EVKLTQGICLEQNKTSSCAEFKKDRGEGLARVLCGEEQADADAGAQVCSL
LLAQSEVRPQCLLLVLANRTEISSKLQLMKKHQSDLKKLGILDFTEQDVA
SHQSYSQKT

Since CARs are typically homodimers (see FIG. 1a), cross-pairing may result in a heterodimeric chimeric antigen receptor. This is undesirable for various reasons, for example: (1) the epitope may not be at the same “level” on the target cell so that a cross-paired CAR may only be able to bind to one antigen; (2) the VH and VL from the two different scFv could swap over and either fail to recognize target or worse recognize an unexpected and unpredicted antigen. For the “AND NOT” gate, the spacer of the first CAR may be sufficiently different from the spacer of the second CAR in order to avoid cross-pairing. The amino acid sequence of the first spacer may share less that 50%, 40%, 30% or 20% identity at the amino acid level with the second spacer.

In the AND NOT gate of the invention, it is important that the spacer be sufficiently different as to prevent cross-pairing, but to be sufficiently similar to co-localise. Pairs of orthologous spacer sequences may be employed. Examples are murine and human CD8 stalks, or alternatively spacer domains which are monomeric—for instance the ectodomain of CD2.

Examples of spacer pairs which co-localise and are therefore suitable for use with the AND NOT gate are shown in the following Table:

| Stimulatory CAR spacer | Inhibitory CAR spacer |
|---|---|
| Human-CD8aSTK | Mouse CD8aSTK |
| Human-CD28STK | Mouse CD8aSTK |
| Human-IgG-Hinge | Human-CD3z ectodomain |
| Human-CD8aSTK | Mouse CD28STK |
| Human-CD28STK | Mouse CD28STK |
| Human-IgG-Hinge-CH2CH3 | Human-IgM-Hinge-CH2CH3CD4 |

The relative dimensions of some commonly used spacers are illustrated in FIG. 13. FIG. 14 shows a matrix of spacer pairs and their suitability for use with an AND NOT gate.

All the spacer domains mentioned above form homodimers. However the mechanism is not limited to using homodimeric receptors and should work with monomeric receptors as long as the spacer is sufficiently rigid. An example of such a spacer is CD2 or truncated CD22.

Transmembrane Domain

The transmembrane domain is the sequence of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, which gives good receptor stability.

Activating Endodomain

The endodomain is the signal-transmission portion of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

In the “AND NOT gate” of the present invention, the cell comprises at least two CARs, at least one of which is an activating CAR which comprises or associates with an activating endodomain. An activating endodomain may, for example, comprise the CD3-Zeta endodomain alone, the CD3-Zeta endodomain with that of either CD28 or OX40 or the CD28 endodomain and OX40 and CD3-Zeta endodomain.

Any endodomain which contains an ITAM motif can act as an activation endodomain in this invention. Several proteins are known to contain endodomains with one or more ITAM motifs. Examples of such proteins include the CD3 epsilon chain, the CD3 gamma chain and the CD3 delta chain to name a few. The ITAM motif can be easily recognized as a tyrosine separated from a leucine or isoleucine by any two other amino acids, giving the signature YxxL/I. Typically, but not always, two of these motifs are separated by between 6 and 8 amino acids in the tail of the molecule (YxxL/Ix(6-8)YxxL/I). Hence, one skilled in the art can readily find existing proteins which contain one or more ITAM to transmit an activation signal. Further, given the motif is simple and a complex secondary structure is not required, one skilled in the art can design polypeptides containing artificial ITAMs to transmit an activation signal (see WO 2000063372, which relates to synthetic signalling molecules).

The transmembrane and intracellular T-cell signalling domain (endodomain) of a CAR with an activating endodomain may comprise the sequence shown as SEQ ID No. 13, 14 or 15 or a variant thereof having at least 80% sequence identity.

SEQ ID No. 13 comprising CD28 transmembrane domain
and CD3 Z endodomain
FWVLVVVGGVLACYSLLVTVAFIIFWVRRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPR
SEQ ID No. 14 comprising CD28 transmembrane domain
and CD28 and CD3 Zeta endodomains
FWVLVVVGGVLACYSLLVTVAFIIFWVRSKRSRLLHSDYMNMTPRRPGPT
RKHYQPYAPPRDFAAYRSRVKFSRSADAPAYQQGQNQLYNELNLGRREEY
DVLDKRRGRDPEMGGKPRRKNPQEGLYNELQKDKMAEAYSEIGMKGERRR
GKGHDGLYQGLSTATKDTYDALHMQALPPR
SEQ ID No. 15 comprising CD28 transmembrane domain
and CD28, OX40 and CD3 Zeta endodomains.
FWVLVVVGGVLACYSLLVTVAFIIFWVRSKRSRLLHSDYMNMTPRRPGPT
RKHYQPYAPPRDFAAYRSRDQRLPPDAHKPPGGGSFRTPIQEEQADAHST
LAKIRVKFSRSADAPAYQQGQNQLYNELNLGRREEYDVLDKRRGRDPEMG
GKPRRKNPQEGLYNELQKDKMAEAYSEIGMKGERRRGKGHDGLYQGLSTA
TKDTYDALHMQALPPR

A variant sequence may have at least 80%, 85%, 90%, 95%, 98% or 99% sequence identity to SEQ ID No. 13, 14 or 15, provided that the sequence provides an effective trans-membrane domain and an effective intracellular T cell signaling domain.

“Ligation-ON” Endodomain

In the AND NOT gate of the present invention, one of the CARs comprises a “ligation-on” inhibitory endodomain such that the inhibitory CAR does not significantly inhibit T-cell activation by the activating CAR in the absence of inhibitory CAR ligation, but inhibits T-cell activation by the activating CAR when the inhibitory CAR is ligated.

The “ligation-on” inhibitory endodomain may be or comprise a tyrosine phosphatase that is incapable of inhibiting the TCR signalling when only the stimulatory receptor is ligated.

The “ligation-on” inhibitory endodomain may be or comprise a tyrosine phosphatase with a sufficiently slow catalytic rate for phosphorylated ITAMs that is incapable of inhibiting the TCR signalling when only the stimulatory receptor is ligated but it is capable of inhibiting the TCR signalling response when concentrated at the synapse. Concentration at the synapse is achieved through inhibitory receptor ligation.

If a tyrosine phosphatase has a catalytic rate which is too fast for a “ligation-on” inhibitory endodomain, then it is possible to tune-down the catalytic rates of phosphatase through modification such as point mutations and short linkers (which cause steric hindrance) to make it suitable for a “ligation-on” inhibitory endodomain.

In this first embodiment the endodomain may be or comprise a phosphatase which is considerably less active than CD45 or CD148, such that significant dephosphorylation of ITAMS only occurs when activating and inhibitory endodomains are co-localised. Many suitable sequences are known in the art. For example, the inhibitory endodomain of a NOT AND gate may comprise all or part of a protein-tyrosine phosphatase such as PTPN6.

Protein tyrosine phosphatases (PTPs) are signaling molecules that regulate a variety of cellular processes including cell growth, differentiation, mitotic cycle, and oncogenic transformation. The N-terminal part of this PTP contains two tandem Src homolog (SH2) domains, which act as protein phospho-tyrosine binding domains, and mediate the interaction of this PTP with its substrates. This PTP is expressed primarily in hematopoietic cells, and functions as an important regulator of multiple signaling pathways in hematopoietic cells.

The inhibitor domain may comprise all of PTPN6 (SEQ ID No. 16) or just the phosphatase domain (SEQ ID No. 17).

sequence of PTPN6

SEQ ID 16

MVRWFHRDLSGLDAETLLKGRGVHGSFLARPSRKNQGDFSLSVRVGDQVT
HIRIQNSGDFYDLYGGEKFATLTELVEYYTQQQGVLQDRDGTIIHLKYPL
NCSDPTSERWYHGHMSGGQAETLLQAKGEPWTFLVRESLSQPGDFVLSVL
SDQPKAGPGSPLRVTHIKVMCEGGRYTVGGLETFDSLTDLVEHFKKTGIE
EASGAFVYLRQPYYATRVNAADIENRVLELNKKQESEDTAKAGFWEEFES
LQKQEVKNLHQRLEGQRPENKGKNRYKNILPFDHSRVILQGRDSNIPGSD
YINANYIKNQLLGPDENAKTYIASQGCLEATVNDFWQMAWQENSRVIVMT
TREVEKGRNKCVPYWPEVGMQRAYGPYSVTNCGEHDTTEYKLRTLQVSPL
DNGDLIREIWHYQYLSWPDHGVPSEPGGVLSFLDQINQRQESLPHAGPII
VHCSAGIGRTGTIIVIDMLMENISTKGLDCDIDIQKTIQMVRAQRSGMVQ
TEAQYKFIYVAIAQFIETTKKKLEVLQSQKGQESEYGNITYPPAMKNAHA
KASRTSSKHKEDVYENLHTKNKREEKVKKQRSADKEKSKGSLKRK
sequence of phosphatase domain of PTPN6

SEQ ID 17

FWEEFESLQKQEVKNLHQRLEGQRPENKGKNRYKNILPFDHSRVILQGRD
SNIPGSDYINANYIKNQLLGPDENAKTYIASQGCLEATVNDFWQMAWQEN
SRVIVMTTREVEKGRNKCVPYWPEVGMQRAYGPYSVTNCGEHDTTEYKLR
TLQVSPLDNGDLIREIWHYQYLSWPDHGVPSEPGGVLSFLDQINQRQESL
PHAGPIIVHCSAGIGRTGTIIVIDMLMENISTKGLDCDIDIQKTIQMVRA
QRSGMVQTEAQYKFIYVAIAQF

A second embodiment of a ligation-on inhibitory endodomain is an ITIM (Immunoreceptor Tyrosine-based Inhibition motif) containing endodomain such as that from CD22, LAIR-1, the Killer inhibitory receptor family (KIR), LILRB1, CTLA4, PD-1, BTLA etc. When phosphorylated, ITIMs recruits endogenous PTPN6 through its SH2 domain. If co-localised with an ITAM containing endodomain, dephosphorylation occurs and the activating CAR is inhibited.

An ITIM is a conserved sequence of amino acids (S/IN/LxYxxl/V/L) that is found in the cytoplasmic tails of many inhibitory receptors of the immune system. One skilled in the art can easily find protein domains containing an ITIM. A list of human candidate ITIM-containing proteins has been generated by proteome-wide scans (Staub, et al (2004) Cell. Signal. 16, 435-456). Further, since the consensus sequence is well known and little secondary structure appears to be required, one skilled in the art could generate an artificial ITIM.

ITIM endodomains from PDCD1, BTLA4, LILRB1, LAIR1, CTLA4, KIR2DL1, KIR2DL4, KIR2DL5, KIR3DL1 and KIR3DL3 are shown in SEQ ID 18 to 27 respectively

PDCD1 endodomain

SEQ ID No. 18

CSRAARGTIGARRTGQPLKEDPSAVPVFSVDYGELDFQWREKTPEPPVPC
VPEQTEYATIVFPSGMGTSSPARRGSADGPRSAQPLRPEDGHCSWPL
BTLA4

SEQ ID No. 19

KLQRRWKRTQSQQGLQENSSGQSFFVRNKKVRRAPLSEGPHSLGCYNPMM
EDGISYTTLRFPEMNIPRTGDAESSEMQRPPPDCDDTVTYSALHKRQVGD
YENVIPDFPEDEGIHYSELIQFGVGERPQAQENVDYVILKH
LILRB1

SEQ ID No. 20

LRHRRQGKHWTSTQRKADFQHPAGAVGPEPTDRGLQWRSSPAADAQEENL
YAAVKHTQPEDGVEMDTRSPHDEDPQAVTYAEVKHSRPRREMASPPSPLS
GEFLDTKDRQAEEDRQMDTEAAASEAPQDVTYAQLHSLTLRREATEPPPS
QEGPSPAVPSIYATLAIH
LAIR1

SEQ ID No. 21

HRQNQIKQGPPRSKDEEQKPQQRPDLAVDVLERTADKATVNGLPEKDRET
DTSALAAGSSQEVTYAQLDHWALTQRTARAVSPQSTKPMAESITYAAV
ARH
CTLA4

SEQ ID No. 22

FLLWILAAVSSGLFFYSFLLTAVSLSKMLKKRSPLTTGVYVKMP
PTEPECEKQFQPYFIPIN
KIR2DL1

SEQ ID No. 23

GNSRHLHVLIGTSVVIIPFAILLFFLLHRWCANKKNAVVMDQEPAGNRTV
NREDSDEQDPQEVTYTQLNHCVFTQRKITRPSQRPKTPPTDIIVYTELPN
AESRSKVVSCP
KIR2DL4

SEQ ID No. 24

GIARHLHAVIRYSVAIILFTILPFFLLHRWCSKKKENAAVMNQE
PAGHRTVNREDSDEQDPQEVTYAQLDHCIFTQRKITGPSQRSKRPSTDTS
VCIELPNAEPRALSPAHEHHSQALMGSSRETTALSQTQLASSNVPAAGI
KIR2DL5

SEQ ID No. 25

TGIRRHLHILIGTSVAIILFIILFFFLLHCCCSNKKNAAVMDQEPAGDRT
VNREDSDDQDPQEVTYAQLDHCVFTQTKITSPSQRPKTPPTDTTMYMELP
NAKPRSLSPAHKHHSQALRGSSRETTALSQNRVASSHVPAAGI
KIR3DL1

SEQ ID No. 26

KDPRHLHILIGTSVVIILFILLLFFLLHLWCSNKKNAAVMDQEPAGNRTA
NSEDSDEQDPEEVTYAQLDHCVFTQRKITRPSQRPKTPPTDTILYTELPN
AKPRSKVVSCP
KIR3DL3

SEQ ID No. 27

KDPGNSRHLHVLIGTSVVIIPFAILLFFLLHRWCANKKNAVVMDQEPAGN
RTVNREDSDEQDPQEVTYAQLNHCVFTQRKITRPSQRPKTPPTDTSV

A third embodiment of a ligation-on inhibitory endodomain is an ITIM containing endodomain co-expressed with a fusion protein. The fusion protein may comprise at least part of a protein-tyrosine phosphatase and at least part of a receptor-like tyrosine phosphatase. The fusion may comprise one or more SH2 domains from the protein-tyrosine phosphatase. For example, the fusion may be between a PTPN6 SH2 domain and CD45 endodomain or between a PTPN6 SH2 domain and CD148 endodomain. When phosphorylated, the ITIM domains recruit the fusion protein bring the highly potent CD45 or CD148 phosphatase to proximity to the activating endodomain blocking activation.

Sequences of possible fusion proteins are given below as SEQ ID No. 28 and 29

PTPN6-CD45 fusion protein

SEQ ID No. 28

WYHGHMSGGQAETLLQAKGEPWTFLVRESLSQPGDFVLSVLSDQPKAGPG
SPLRVTHIKVMCEGGRYTVGGLETFDSLTDLVEHFKKTGIEEASGAFVYL
RQPYKIYDLHKKRSCNLDEQQELVERDDEKQLMNVEPIHADILLETYKRK
IADEGRLFLAEFQSIPRVFSKFPIKEARKPFNQNKNRYVDILPYDYNRVE
LSEINGDAGSNYINASYIDGFKEPRKYIAAQGPRDETVDDFWRMIWEQKA
TVIVMVTRCEEGNRNKCAEYWPSMEEGTRAFGDVVVKINQHKRCPDYIIQ
KLNIVNKKEKATGREVTHIQFTSWPDHGVPEDPHLLLKLRRRVNAFSNFF
SGPIVVHCSAGVGRTGTYIGIDAMLEGLEAENKVDVYGYVVKLRRQRCLM
VQVEAQYILIHQALVEYNQFGETEVNLSELHPYLHNMKKRDPPSEPSPLE
AEFQRLPSYRSWRTQHIGNQEENKSKNRNSNVIPYDYNRVLKHELEMSKE
SEHDSDESSDDDSDSEEPSKYINASFIMSYWKPEVMIAAQGPLKETIGDF
MIQRKVKVIVMLTELKHGDQEICAQYWGEGKQTYGDIEVDLKDTDKSSTY
TLRVFELRHSKRKDSRTVYQYQYTNWSVEQLPAEPKELISMIQVVKQKLP
QKNSSEGNKHHKSTPLLIHCRDGSQQTGIFCALLNLLESAETEEVVDIFQ
VVKALRKARPGMVSTFEQYQFLYDVIASTYPAQNGQVKKNNHQEDKIEFD
NEVDKVKQDANCVNPLGAPEKLPEAKEQAEGSEPTSGTEGPEHSVNGPAS
PALNQGS
PTPN6-CD148 fusion

SEQ ID No. 29

ETLLQAKGEPWTFLVRESLSQPGDFVLSVLSDQPKAGPGSPLRVTHIKVM
CEGGRYTVGGLETFDSLTDLVEHFKKTGIEEASGAFVYLRQPYRKKRKDA
KNNEVSFSQIKPKKSKLIRVENFEAYFKKQQADSNCGFAEEYEDLKLVGI
SQPKYAAELAENRGKNRYNNVLPYDISRVKLSVQTHSTDDYINANYMPGY
HSKKDFIATQGPLPNTLKDFWRMVWEKNVYAIIMLTKCVEQGRTKCEEYW
PSKQAQDYGDITVAMTSEIVLPEVVTIRDFTVKNIQTSESHPLRQFHFTS
WPDHGVPDTTDLLINFRYLVRDYMKQSPPESPILVHCSAGVGRTGTFIAI
DRLIYQIENENTVDVYGIVYDLRMHRPLMVQTEDQYVFLNQCVLDIVRSQ
KDSKVDLIYQNTTAMTIYENLAPVTTFGKTNGYIA

A ligation-on inhibitory CAR may comprise all or part of SEQ ID No 16 or 17. It may comprise all or part of SEQ ID 18 to 27. It may comprise all or part of SEQ ID 18 to 27 co-expressed with either SEQ ID 28 or 29. It may comprise a variant of the sequence or part thereof having at least 80% sequence identity, as long as the variant retains the capacity to inhibit T cell signaling by the activating CAR upon ligation of the inhibitory CAR.

As above, alternative spacers and endodomains may be tested for example using the model system exemplified herein. It is shown in Example 5 that the PTPN6 endodomain can function as a semi-inhibitory CAR in combination with an activating CAR containing a CD3 Zeta endodomain. These CARs rely upon a human CD8 stalk spacer on one CAR and a mouse CD8 stalk spacer on the other CAR. The orthologous sequences prevent cross pairing. However, when both receptors are ligated, the similarity between the spacers results in co-segregation of the different receptors in the same membrane compartments. This results in inhibition of the CD3 Zeta receptor by the PTPN6 endodomain. If only the activating CAR is ligated the PTPN6 endodomain is not sufficiently active to prevent T cell activation. In this way, activation only occurs if the activating CAR is ligated and the inhibitory CAR is not ligated (AND NOT gating). It can be readily seen that this modular system can be used to test alternative spacer pairs and inhibitory domains. If the spacers do not achieve co-segregation following ligation of both receptors, the inhibition would not be effective and so activation would occur. If the semi-inhibitory endodomain under test is ineffective, activation would be expected in the presence of ligation of the activating CAR irrespective of the ligation status of the semi-inhibitory CAR.

Cleavage Site

The present nucleic acid construct comprises a sequence encoding a cleavage site positioned between nucleic acid sequences which encode first and second CARs, such that the first and second CARs can be expressed as separate entities.

The cleavage site may be any sequence which enables the polypeptide comprising the first and second CARs to become separated.

The term “cleavage” is used herein for convenience, but the cleavage site may cause the first and second CARs to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode first and second CARs, causes the first and second CARs to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the first and second CARs and the self-cleaving peptide is produced, it is immediately “cleaved” or separated into distinct and discrete first and second CARs without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus.

The C-terminal 19 amino acids of the longer cardiovirus protein, together with the N-terminal proline of 2B mediate “cleavage” with an efficiency approximately equal to the apthovirus FMDV 2a sequence. Cardioviruses include encephalomyocarditis virus (EMCV) and Theiler's murine encephalitis virus (TMEV).

Mutational analysis of EMCV and FMDV 2A has revealed that the motif DxExNPGP is intimately involved in “cleavage” activity (Donelly et al (2001) as above).

The cleavage site of the present invention may comprise the amino acid sequence: Dx₁Ex₂NPGP, where x₁ and x₂ are any amino acid. X₁ may be selected from the following group: I, V, M and S. X₂ may be selected from the following group: T, M, S, L, E, Q and F.

For example, the cleavage site may comprise one of the amino acid sequences shown in Table 6.

TABLE 6

| Motif | Present in: |
|---|---|
| DIETNPGP | Picornaviruses EMCB, |
| | EMCD, EMCPV21 |
| DVETNPGP | Picornaviruses MENGO and |
| | TMEBEAN; Insect virus |
| | DCV, ABPV |
| DVEMNPGP | Picornaviruses TMEGD7 and |
| | TMEBEAN |
| DVESNPGP | Picornaviruses FMDA10, |
| | FMDA12, FMDC1, FMD01K, |
| | FMDSAT3, FMDVSAT2, |
| | ERAV; Insect virus CrPV |
| DMESNPGP | Picornavirus FMDV01G |
| DVELNPGP | Picornavirus ERBV; Porcine |
| | rotavirus |
| DVEENPGP | Picornavirus PTV-1; Insect |
| | virus TaV; Trypanosoma |
| | TSR1 |
| DIELNPGP | Bovine Rotavirus, human |
| | rotavirus |
| DIEQNPGP | Trypanosoma AP |
| | endonuclease |
| DSEFNPGP | Bacterial sequence T. maritima |

The cleavage site, based on a 2A sequence may be, for example 15-22 amino acids in length. The sequence may comprise the C-terminus of a 2A protein, followed by a proline residue (which corresponds to the N-terminal proline of 2B).

Mutational studies have also shown that, in addition to the naturally occurring 2A sequences, some variants are also active. The cleavage site may correspond to a variant sequence from a naturally occurring 2A polypeptide, have one, two or three amino acid substitutions, which retains the capacity to induce the “cleavage” of a polyprotein sequence into two or more separate proteins.

The cleavage sequence may be selected from the following which have all been shown to be active to a certain extent (Donnelly et al (2001) as above):

LLNFDLLKLAGDVESNPGP
LLNFDLLKLAGDVQSNPGP
LLNFDLLKLAGDVEINPGP
LLNFDLLKLAGDVEFNPGP
LLNFDLLKLAGDVESHPGP
LLNFDLLKLAGDVESEPGP
LLNFDLLKLAGDVESQPGP
LLNFDLLKLAGDVESNPGG

Based on the sequence of the DxExNPGP “a motif, “2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above). The cleavage site may comprise one of these 2A-like sequences, such as:

YHADYYKQRLIHDVEMNPGP
HYAGYFADLLIHDIETNPGP
QCTNYALLKLAGDVESNPGP
ATNFSLLKQAGDVEENPGP
AARQMLLLLSGDVETNPGP
RAEGRGSLLTCGDVEENPGP
TRAEIEDELIRAGIESNPGP
TRAEIEDELIRADIESNPGP
AKFQIDKILISGDVELNPGP
SSIIRTKMLVSGDVEENPGP
CDAQRQKLLLSGDIEQNPGP
YPIDFGGFLVKADSEFNPGP

The cleavage site may comprise the 2A-like sequence RAEGRGSLLTCGDVEENPGP.

It has been shown that including an N-terminal “extension” of between 5 and 39 amino acids can increase activity (Donnelly et al (2001) as above). In particular, the cleavage sequence may comprise one of the following sequences or a variant thereof having, for example, up to 5 amino acid changes which retains cleavage site activity:

VTELLYRMKRAETYCPRPLAIHPTEARHKQKIVAPVKQTLNFDLLKLAGD
VESNPGPLLAIHPTEARHKQKIVAPVKQTLNFDLLKLAGDVESNPGP
EARHKQKIVAPVKQTLNFDLLKLAGDVESNPGP
APVKQTLNFDLLKLAGDVESNPGP

Intracellular Retention Signal

The nucleic acid construct of the present invention may comprise a sequence which encodes a CAR comprising an intracellular retention signal.

Protein targeting or protein sorting is the biological mechanism by which proteins are transported to the appropriate destinations in the cell or outside of it. Proteins can be targeted to the inner space of an organelle, different intracellular membranes, plasma membrane, or to exterior of the cell via secretion. This delivery process is carried out based on sequence information contain in the protein itself.

Proteins synthesised in the rough endoplasmic reticulum (ER) of eukaryotic cells use the exocytic pathway for transport to their final destinations. Proteins lacking special sorting signals are vectorially transported from the ER via the Golgi and the trans-Golgi network (TGN) to the plasma membrane. Other proteins have targeting signals for incorporation into specific organelles of the exocytic pathway, such as endosomes and lysosomes.

Lysosomes are acidic organelles in which endogenous and internalised macromolecules are degraded by luminal hydolases. Endogenous macromolecules reach the lysosome by being sorted in the TGN from which they are transported to endosomes and then lysosomes.

The targeting signals used by a cell to sort proteins to the correct intracellular location may be exploited by the present invention. The signals may be broadly classed into the following types:

i) endocytosis signals
ii) Golgi retention signals
iii) TGN recycling signals
iv) ER retention signals
v) lysosomal sorting signals

‘Intracellular retention signal’ refers to an amino acid sequence which directs the protein in which it is encompassed to a cellular compartment other than the cell surface membrane or to the exterior of the cell.

The intracellular retention signal causes a reduction in the amount of the CAR expressed on the surface of a cell compared to an equivalent, control CAR or other transmembrane protein which does not comprise an intracellular retention signal.

In other words, the proportion of translated CAR comprising an intracellular retention signal which is expressed on at the cell surface is less than the proportion of an equivalent amount of an equivalent, translated control CAR or other transmembrane protein which does not comprise an intracellular retention signal.

For example, the amount of the CAR comprising an intracellular retention signal which is expressed on the surface of a cell may be less than 75%, less than 50%, less than 25% or less than 10% of the amount of an equivalent control CAR or other transmembrane protein which does not comprise an intracellular retention signal.

Constructs which express a polyprotein that is subsequently cleaved by a protease are generally limited by the fact the expression of the peptides from the polyprotein is limited to a 1:1 ratio. However, in the present invention, the inclusion of an intracellular retention signal in the CAR means that its expression on the cell surface can be modulated (e.g. reduced compared to an equivalent control CAR or other transmembrane protein which does not comprise an intracellular retention signal). As such the ratio of the CAR which comprises the intracellular retention signal expressed on the cell surface compared to the expression of the second CAR expressed in the polyprotein may be, for example about 1:1.5, of from 1:1.5-1:2, 1:2-1:3, 1:3-1:4, 1:4-1:5, or more than 1:5.

The amount of a CAR expressed on the surface of a cell may be determined using methods which are known in the art, for example flow cytometry or fluorescence microscopy.

The intracellular retention signal may direct the CAR away from the secretory pathway during translocation from the ER.

The intracellular retention signal may direct the CAR to an intracellular compartment or complex. The intracellular retention signal may direct the CAR to a membrane-bound intracellular compartment.

For example, the intracellular retention signal may direct the CAR to a lysosomal, endosomal or Golgi compartment (trans-Golgi Network, ‘TGN’).

Within a normal cell, proteins arising from biogenesis or the endocytic pathway are sorted into the appropriate intracellular compartment following a sequential set of sorting decisions. At the plasma membrane, proteins can either remain at the cell surface or be internalised into endosomes. At the TGN, the choice is between going to the plasma membrane or being diverted to endosomes. In endosomes, proteins can either recycle to the plasma membrane or go to lysosomes. These decisions are governed by sorting signals on the proteins themselves.

Lysosomes are cellular organelles that contain acid hydrolase enzymes that break down waste materials and cellular debris. The membrane around a lysosome allows the digestive enzymes to work at the pH they require. Lysosomes fuse with autophagic vacuoles (phagosomes) and dispense their enzymes into the autophagic vacuoles, digesting their contents.

An endosome is a membrane-bounded compartment inside eukaryotic cells. It is a compartment of the endocytic membrane transport pathway from the plasma membrane to the lysosome and provides an environment for material to be sorted before it reaches the degradative lysosome. Endosomes may be classified as early endosomes, late endosomes, or recycling endosomes depending on the time it takes for endocytosed material to reach them. The intracellular retention signal used in the present invention may direct the protein to a late endosomal compartment.

The Golgi apparatus is part of the cellular endomembrane system, the Golgi apparatus packages proteins inside the cell before they are sent to their destination; it is particularly important in the processing of proteins for secretion.

There is a considerable body of knowledge which has arisen from studies investigating the sorting signals present in known proteins, and the effect of altering their sequence and/or position within the molecule (Bonifacino and Traub (2003) Ann. Rev. Biochem. 72:395-447; Braulke and Bonifacino (2009) Biochimica and Biophysica Acta 1793:605-614; Griffith (2001) Current Biology 11:R226-R228; Mellman and Nelson (2008) Nat Rev Mol Cell Biol. 9:833-845; Dell'Angelica and Payne (2001) Cell 106:395-398; Schafer et al (1995) EMBO J. 14:2424-2435; Trejo (2005) Mol. Pharmacol. 67:1388-1390). Numerous studies have shown that it is possible to insert one or more sorting signals into a protein of interest in order to alter the intracellular location of a protein of interest (Pelham (2000) Meth. Enzymol. 327:279-283).

It is therefore perfectly possible to select a sorting signal having a desired localisation property and include it within a protein of interest in order to direct the intracellular location of that protein. In connection with the present application, it is therefore possible to select a sorting signal having the desired amount of reduction of expression at the plasma membrane.

The optimal position of the sorting signal in the nascent protein of interest may depend on the type of transmembrane protein (i.e. types I-IV) and whether the C-terminus is on the luminal or the cytoplasmic side of the membrane (Goder and Spiess (2001) FEBS Lett 504:87-93). This may readily be determined by considering the position of the sorting signal in its natural protein.

Examples of endocytosis signals include those from the transferrin receptor and the asialoglycoprotein receptor.

Examples of signals which cause TGN-endosome recycling include those form proteins such as the CI- and CD-MPRs, sortilin, the LDL-receptor related proteins LRP3 and LRP10 and β-secretase, GGA1-3, LIMP-II, NCP1, mucolipn-1, sialin, GLUT8 and invariant chain.

Examples of TGN retention signals include those from the following proteins which are localized to the TGN: the prohormone processing enzymes furin, PC7, CPD and PAM; the glycoprotein E of herpes virus 3 and TGN38.

Examples of ER retention signals include C-terminal signals such as KDEL, KKXX or KXKXX and the RXR(R) motif of potassium channels. Known ER proteins include the adenovirus E19 protein and ERGIC53.

Examples of lysosomal sorting signals include those found in lysosomal membrane proteins, such as LAMP-1 and LAMP-2, CD63, CD68, endolyn, DC-LAMP, cystinosin, sugar phosphate exchanger 2 and acid phosphatase.

Tunability

The relative expression of one or both CARs may be fine tuned using the method of the invention by various methods, such as
a) altering the position of the intracellular retention signal in the protein molecule; and/or
b) selecting a particular intracellular retention signal.

Option a) is discussed in more detail below.

With regard to option b), a range of intracellular retention signals is available from the large number of naturally occurring proteins which are sorted to distinct cellular locations inside eukaryotic cells. It is also possible to use “synthetic” intracellular retention signals which comprise one or more of the motifs found in naturally occurring proteins (see next section) and have a similar sorting signal function.

A cascade of signal strength is available, depending on the intracellular location to which the sorting signal sends the relevant protein. Broadly speaking, the more “intracellular” the location directed by the sorting signal, the “stronger” the signal is in terms of lowering the relative expression of the protein.

When a sorting signal directs a protein to the lysosomal compartment, the protein is internalised and degraded by the cell, resulting in relatively little escape to the cell surface. The protein is degraded and lost from the system once it enters the lysosome. Therefore lysosomal sorting signals, such as LAMP1, are the “strongest” in terms of reducing relative expression at the cell surface.

When a sorting signal directs a protein to be retained in the ER, only a very small proportion of the protein gets to the cell surface. Hence ER retention or recycling signals, such as ER-GIC-53 and KKFF signal are the next most strong, in terms of reducing relative expression at the cell surface.

When a sorting signal directs a protein to the endosomal, Golgi or TGN compartments, then the protein is likely to recycle to some extent between the TGN, the endosomal compartment, and the plasma membrane. These signals provide a more limited level of reduction of expression as a significant proportion of the protein will still reach the plasma membrane.

In general the reduction in expression seen with known sorting signals can be summarised as follows:

Lysosomal sorting signals>ER retention/recycling signals>TGN retention/recycling signals>endocytosis signals.

The tunability using different sorting signals and/or different positions of sorting signals within the protein is especially useful when one considers the expression of multiple proteins, each with their own relative expression. For example, consider a nucleic acid construct having the following structure:

A-X-B-Y-C
in which
   A, B and C are nucleic acid sequences encoding polypeptides; and
   X and Y are nucleic acid sequences encoding cleavage sites.

The nucleic acid construct will encode three proteins A, B and C, any or all of which may be CARs. For example, B and C may be CARs which comprise an intracellular retention signal. If it is desired for A, B and C to be expressed such that the relative levels are A>B>C, then the nucleic acid sequence A may have no intracellular retention signal, the nucleic acid sequence B may have an intracellular retention signal that causes a small proportion of protein B to be retained in the cell (i.e. not to be expressed at the cell surface), and the nucleic acid sequence C may have an intracellular retention signal that causes a large proportion of protein C to be retained in the cell.

As explained below, differential amounts of intracellular retention, leading to different amounts of cell surface expression may be achieved by:

(a) using different intracellular retention signals for the proteins; and/or
(b) having the intracellular retention signal located at a different position in the proteins.

Signal Types

Numerous proteins which include an intracellular retention signal and are directed to an intracellular compartment are known in the art.

The intracellular retention signal may be a retention signal from a protein which resides in the lysosomal, endosomal or Golgi compartment.

Intracellular retention signals are well known in the art (see, for example, Bonifacino & Traub; Annu. Rev. Biochem.; 2003; 72; 395-447).

The intracellular retention signal may be a tyrosine-based sorting signal, a dileucine-based sorting signal, an acidic cluster signal, a lysosomal avoidance signal, an NPFX′(1,2)D-Type signal, a KDEL, a KKX′X′ or a KX′KX′X′ signal (wherein X′ is any amino acid).

Tyrosine-based sorting signals mediate rapid internalization of transmembrane proteins from the plasma membrane and the targeting of proteins to lysosomes (Bonifacino & Traub; as above). Two types of tyrosine-based sorting signals are represented by the NPX′Y and YX′X′Z′ consensus motifs (wherein Z′ is an amino acid with a bulky hydrophobic side chain).

NPX′Y signals have been shown to mediate rapid internalization of type I transmembrane proteins, they occur in families such as members of the LDL receptor, integrin β, and β-amyloid precursor protein families.

Examples of NPX′Y signals are provided in Table 7.

TABLE 7
NPX′Y signals

| Protein | Species | Sequence |
|---|---|---|
| LDL receptor | Human | Tm-10-INFDNPVYQKTT-29 |
| LRP1 (1) | Human | Tm-21-VEIGNPTYKMYE-64 |
| LRP1 (2) | Human | Tm-55-TMFTNPVYATLY-33 |
| LRP1 | Drosophila | Tm-43-GNFANPVYESMY-38 |
| LRP1 (1) | C. elegans | Tm-54-TTFTNPVYELED-91 |
| LRP1 (2) | C. elegans | Tm-140-LRVDNPLYDPDS-4 |
| Megalin (1) | Human | Tm-70-IIFENPMYSARD-125 |
| Megalin (2) | Human | Tm-144-TNFENPIYAQME-53 |
| Integrin 13-1 | Human | Tm-18-DTGENPIYKSAV-11 |
| (1) | | |
| Integrin 13-1 | Human | Tm-30-TTVVNPKYEGK |
| (2) | | |
| Integrin 13 (1) | Drosophila | Tm-26-WDTENPIYKQAT-11 |
| Integrin 13 (2) | Drosophila | Tm-35-STFKNPMYAGK |
| APLP1 | Human | Tm-33-HGYENPTYRFLE-3 |
| APP | Human | Tm-32-NGYENPTYKFFE-4 |
| APP-like | Drosophila | Tm-38-NGYENPTYKYFE-3 |
| Insulin receptor | Human | Tm-36-YASSNPEYLSAS-379 |
| EGR receptor (1) | Human | Tm-434-GSVQNPVYHNQP-96 |
| EGR receptor (2) | Human | Tm-462-TAVGNPEYLNTV-68 |
| EGR receptor (3) | Human | Tm-496-1SLDNPDYQQDF-34 |

Numbers in parentheses indicate motifs that are present in more than one copy within the same protein. The signals in this and other tables should be considered examples. Key residues are indicated in bold type. Numbers of amino acids before (i.e., amino-terminal) and after (i.e., carboxy-terminal) the signals are indicated. Abbreviations: Tm, transmembrane; LDL, low density lipoprotein; LRP1, LDL receptor related protein 1; APP, 13-amyloid precursor protein; APLP1, APP-like protein 1.

YX′X′Z′-type signals are found in endocytic receptors such as the transferrin receptor and the asialoglycoprotein receptor, intracellular sorting receptors such as the CI- and CD-MPRs, lysosomal membrane proteins such as LAMP-1 and LAMP-2, and TGN proteins such as TGN38 and furin, as well as in proteins localized to specialized endosomal-lysosomal organelles such as antigen-processing compartments (e.g., HLA-DM) and cytotoxic granules (e.g., GMP-17). The YX′X′Z′-type signals are involved in the rapid internalization of proteins from the plasma membrane. However, their function is not limited to endocytosis, since the same motifs have been implicated in the targeting of transmembrane proteins to lysosomes and lysosome-related organelles.

Examples of YX′X′Z′-type signals are provided in Table 8.

TABLE 8
YX′X′Z′-type signals

| Protein | Species | Sequence |
|---|---|---|
| LAMP-1 | Human | Tm-RKRSHAGYQTI |
| LAMP-2a | Human | Tm-KHHHAGYEQF |
| LAMP-2a | Chicken | Tm-KKHHNTGYEQF |
| LAMP-2b | Chicken | Tm-RRKSRTGYQSV |
| LAMP-2c | Chicken | Tm-RRKSYAGYQTL |
| LAMP | Drosophila | Tm-RRPSTSRGYMSF |
| LAMP | Earthworm | Tm-RKRSRRGYESV |
| CD63 | Human | Tm-KSIRSGYEVM |
| GMP-17 | Human | Tm-HCGGPRPGYETL |
| GMP-17 | Mouse | Tm-HCRTRRAEYETL |
| CD68 | Human | Tm-RRRPSAYQAL |
| CD1b | Human | Tm-RRRSYQNIP |
| CD1c | Human | Tm-KKHCSYQDIL |
| CD1d | Mouse | Tm-RRRSAYQDIR |
| CD1 | Rat | Tm-RKRRRSYQDIM |
| Endolyn | Rat | Tm-KFCKSKERNYHTL |
| Endolyn | Drosophila | Tm-KFYKARNERNYHTL |
| TSC403 | Human | Tm-KIRLRCQSSGYQRI |
| TSC403 | Mouse | Tm-KIRQRHQSSAYQRI |
| Cystinosin | Human | Tm-HFCLYRKRPGYDQLN |
| Putative | Human | Tm-12-SLSRGSGYKEI |
| solute carrier | | |
| TRP-2 | Human | Tm-RRLRKGYTPLMET-11 |
| HLA-DM [symbol] | Human | Tm-RRAGHSSYTPLPGS-9 |
| LmpA | Dictyostelium | Tm-KKLRQQKQQGYQAIINNE |
| Putative | Dictyostelium | Tm-RSKSNQNQSYNLIQL |
| lysosomal | | |
| protein | | |
| LIMP-II | Dictyostelium | Tm-RKTFYNNNQYNGYNIIN |
| Transferrin | Human | 16-PLSYTRFSLA-35- |
| receptor | | |
| Asialoglyco- | Human | MTKEYQDLQHL-29- |
| protein | | |
| receptor | | |
| CI-MPR | Human | Tm-22-SYKYSKVNKE-132 |
| CD-MPR | Human | Tm-40-PAAYRGVGDD-16 |
| CTLA-4 | Human | Tm-10-TGVYVKMPPT-16 |
| Furin | Human | Tm-17-LISYKGLPPE-29 |
| TGN38 | Rat | Tm-23-ASDYQRLNLKL |
| gp41 | HIV-1 | Tm-13-RQGYSPLSFQT- |
| Acid | Human | Tm-RMQAQPPGYRHVADGEDHA |
| phosphatase | | |

Dileucine-based sorting signals ([DE]X′X′X′LL[LI]) play critical roles in the sorting of many type I, type II, and multispanning transmembrane proteins. Dileucine-based sorting signals are involved in rapid internalization and lysosomal degradation of transmembrane proteins and the targeting of proteins to the late endosomal-lysosomal compartments. Transmembrane proteins that contain constitutively active forms of this signal are mainly localised to the late endosomes and lysosomes. See legend to Table 7 for explanation of signal format

Examples of [DE]X′X′X′LL[LI] sorting signals are provided in Table 9.

TABLE 9
[DE]X′X′X′LL[LI] sorting signals

| Protein | Species | Signal |
|---|---|---|
| CD3-′Y | Human | Tm-8-SDKQTLLPN-26 |
| LIMP-II | Rat | Tm-11-DERAPLIRT |
| Nmb | Human | Tm-37-QEKDPLLKN-7 |
| QNR-71 | Quail | Tm-37-TERNPLLKS-5 |
| Pmel17 | Human | Tm-33-GENSPLLSG-3 |
| Tyrosinase | Human | Tm-8-EEKQPLLME-12 |
| Tyrosinase | Medaka fish | Tm-16-GERQPLLQS-13 |
| Tyrosinase | Chicken | Tm-8-PEIQPLLTE-13 |
| TRP-1 | Goldfish | Tm-7-EGRQPLLGD-15 |
| TRP-1 | Human | Tm-7-EANQPLLTD-20 |
| TRP-1 | Chicken | Tm-7-ELHQPLLTD-20 |
| TRP-2 | Zebrafish | Tm-5-REFEPLLNA-11 |
| VMAT2 | Human | Tm-6-EEKMAILMD-2 9 |
| VMAT1 | Human | Tm-6-EEKLAILSQ-32 |
| VAchT | Mouse | Tm-10-SERDVLLDE-42 |
| VAMP4 | Human | 19-SERRNLLED-88-Tm |
| Neonatal | Rat | Tm-16-DDSGDLLPG-19 |
| FcR | | |
| CD4 | Human | Tm-12-SQIKRLLSE-17 |
| CD4 | Cat | Tm-12-SHIKRLLSE-17 |
| GLUT4 | Mouse | Tm-17-RRTPSLLEQ-17 |
| GLUT4 | Human | Tm-17-HRTPSLLEQ-17 |
| IRAP | Rat | 46-EPRGSRLLVR-53-Tm |
| Ii | Human | MDDQRDLISNNEQLPMLGR-11-Tm |
| Ii | Mouse | MDDQRDLISNHEQLPILGN-10-Tm |
| Ii | Chicken | MAEEQRDLISSDGSSGVLPI-12-Tm |
| Ii-1 | Zebrafish | MEPDHQNESLIQRVPSAETILGR- |
| | | 12-Tm |
| Ii-2 | Zebrafish | MSSEGNETPLISDQSSVNMGPQP- |
| | | 8-Tm |
| Lamp | Trypanosome | Tm- |
| | | RPRRRTEEDELLPEEAEGLIDPQN |
| Menkes | Human | Tm-74-PDKHSLLVGDFREDDDTAL |
| protein | | |
| NPC1 | Human | Tm-13-TERERLLNF |
| AQP4 | Human | Tm-32-VETDDLIL-29 |
| RME-2 | C. elegans | Tm-104-FENDSLL |
| Vam3p | S. cerevisiae | 153-NEQSPLLHN-121-Tm |
| ALP | S. cerevisiae | 7-SEQTRLVP-18-Tm |
| Gap1p | S. cerevisiae | Tm-23-EVDLDLLK-24 |

DX′X′LL signals constitute a distinct type of dileucine-based sorting signals. These signals are present in several transmembrane receptors and other proteins that cycle between the TGN and endosomes, such as the CI- and CD-MPRs, sortilin, the LDL-receptor-related proteins LRP3 and LRP10, and β-secretase. See legend to Table 7 for explanation of signal format.

Examples of DX′X′LL sorting signals are provided in Table 10.

TABLE 10
DX′X′LL sorting signals

| Protein | Species | Sequence |
|---|---|---|
| CI-MPR | Human | Tm-151-SFHDDSDEDLLHI |
| CI-MPR | Bovine | Tm-150-TFHDDSDEDLLHV |
| CI-MPR | Rabbit | Tm-151-SFHDDSDEDLLNI |
| CI-MPR | Chicken | Tm-148-SFHDDSDEDLLNV |
| CD-MPR | Human | Tm-54-EESEERDDHLLPM |
| CD-MPR | Chicken | Tm-54-DESEERDDHLLPM |
| Sortilin | Human | Tm-41-GYHDDSDEDLLE |
| SorLA | Human | Tm-41-ITGFSDDVPMVIA |
| Head-activator BP | Hydra | Tm-41-INRFSDDEPLVVA |
| LRP3 | Human | Tm-237-MLEASDDEALLVC |
| ST7 | Human | Tm-330-KNETSDDEALLLC |
| LRP10 | Mouse | Tm-235-WVVEAEDEPLLA |
| LRP10 | Human | Tm-237-WVAEAEDEPLLT |
| Beta-secretase | Human | Tm-9-HDDFADDISLLK |
| Mucolipin-1 | Mouse | Tm-43-GRDSPEDHSLLYN |
| Nonclassical MHC-I | Deer mouse | Tm-6-VRCHPEDDRLLG |
| FLJ30532 | Human | Tm-83-HRVSQDDLDLLTS |
| GGA1 | Human | 350-ASVSLLDDELMSL-275 |
| GGA1 | Human | 415-ASSGLDDLDLLGK-211 |
| GGA2 | Human | 408-VQNPSADRNLLDL-192 |
| GGA3 | Human | 384-NALSWLDEELLCL-326 |
| GGA | Drosophila | 447-TVDSIDDVPLLSD-116 |

Another family of sorting motifs is provided by clusters of acidic residues containing sites for phosphorylation by CKII. This type of motif is often found in transmembrane proteins that are localized to the TGN at steady state, including the prohormone-processing enzymes furin, PC6B, PC7, CPD, and PAM, and the glycoprotein E of herpes virus 3. See legend to Table 7 for explanation of signal format. Serine and threonine residues are underlined.

Examples of acidic cluster signals are provided in Table 11.

TABLE 11
Acidic cluster sorting signals

| Protein | Species | Sequence |
|---|---|---|
| Furin | Mouse | Tm-31-QEECPSDSEEDEG-14 |
| PC6B (1)^{a} | Mouse | Tm-39-RDRDYDEDDEDDI-36 |
| PC6B (2) | Mouse | Tm-69-LDETEDDELEYDDES-4 |
| PC7 | Human | Tm-38-KDPDEVETES-47 |
| CPD | Human | Tm-36-HEFQDETDTEEET-6 |
| PAM | Human | Tm-59-QEKEDDGSESEEEY-12 |
| VMAT2 | Human | Tm-35-GEDEESESD |
| VMAT1 | Human | Tm-35-GEDSDEEPDHEE |
| VAMP4 | Human | 25-LEDDSDEEEDF-81-Tm |
| Glycoprotein B | HCMV | Tm-125-KDSDEEENV |
| Glycoprotein E | Herpes virus 3 | Tm-28-FEDSESTDTEEEF-21 |
| Nef | HIV-1 (AAL65476) | 55-LEAQEEEEV-139 |
| Kex1p (1) | S. cerevisiae | Tm-29-ADDLESGLGAEDDLEQDEQLEG-40 |
| Kex1p (2) | S. cerevisiae | Tm-79-TEIDESFEMTDF |
| Kex2p | S. cerevisiae | Tm-36-TEPEEVEDFDFDLSDEDH-61 |
| Vps10p | S. cerevisiae | Tm-112-FEIEEDDVPTLEEEH-37 |

The KDEL receptor binds protein in the ER-Golgi intermediate compartment, or in the early Golgi and returns them to the ER. Although the common mammalian signal is KDEL, it has been shown that the KDEL receptor binds the sequence HDEL more tightly (Scheel et al; J. Biol. Chem. 268; 7465 (1993)). The intracellular retention signal may be HDEL. See legend to Table 7 for explanation of signal format. Serine and threonine residues are underlined.^{a}The number in parentheses is the motif number.

KKX′X′ and KX′KX′X′ signals are retrieval signals which can be placed on the cytoplasmic side of a type I membrane protein. Sequence requirements of these signals are provided in detail by Teasdale & Jackson (Annu. Rev. Cell Dev. Biol.; 12; 27 (1996)).

The intracellular retention signal may be selected from the group of: NPX′Y, YX′X′Z, [DE]X′X′X′[LI], DX′X′LL, DP[FW], FX′DX′F, NPF, LZX′Z[DE], LLDLL, PWDLW, KDEL, HDEL, KKX′X′ or KX′KX′X′; wherein X′ is any amino acid and Z′ is an amino acid with a bulky hydrophobic side chain.

The intracellular retention signal may be any sequence shown in Tables 7 to 11.

The intracellular retention signal may comprise the Tyrosinase-related protein (TYRP)-1 intracellular retention signal. The intracellular retention signal may comprise the TYRP-1 intracellular domain. The intracellular retention signal may comprise the sequence NQPLLTD (SEQ ID No. 1).

TYRP1 is a well-characterized melansomal protein which is retained in the melanosome (a specialized lysosome) at >99% efficiency. TYRP1 is a 537 amino acid transmembrane protein with a lumenal domain (1-477aa), a transmembrane domain (478-501), and a cytoplasmic domain (502-537). A di-leucine signal residing on the cytoplasmic domain causes retention of the protein. This di-leucine signal has the sequence shown as SEQ ID No. 1 (NQPLLTD).

The intracellular retention signal may be in the endodomain of the CAR. In other words, the intracellular retention signal may be in the domain of the transmembrane protein which would be on the intracellular side of the cell membrane if the protein was correctly expressed at the cell surface.

The intracellular retention signal may be proximal to the transmembrane domain, for instance being immediately connected to it. The intracellular retention signal may be distal to the transmembrane domain—for instance at the carboxy-terminus of the endodomain. The positioning of the retention signal modulates its activity allowing “tuning” of the relative expression of two proteins. For instance in the case of the TYRP1 di-leucine motif, proximal placement results in low-level surface expression, while distal placement results in intermediate surface expression, as shown in the Examples.

Polypeptide of Interest

Any or all of A or B; or A, B or C of the nucleic acid sequences in the constructs defined herein may encode a CAR which may or may not comprise an intracellular retention signal and/or an altered signal peptide.

The nucleic acid construct may comprise one or more further nucleic acid sequence(s) which encode polypeptide of interest (POIs). For example, the POI(s) may be an intracellular protein such as a nucleic protein, a cytoplasmic protein or a protein localised to a membrane-bound compartment; a secretory protein or a transmembrane protein.

The POI may be a suicide gene and/or a marker gene.

Suicide/Marker Gene

A suicide gene is a gene encoding a polypeptide which, when expressed by a cell enables that cell to be deleted.

A marker gene is a gene encoding a polypeptide which enables selection of a cell expressing that polypeptide.

Various suicide and marker genes are known in the art. WO2013/153391 describes compact polypeptide which comprises both a marker moiety and a suicide moiety. The polypeptide may be co-expressed with a therapeutic transgene, such as a gene encoding a CAR.

The marker moiety comprises a minimal epitope of CD34 which allows efficient selection of transduced cells using, for example, the Miltenyi CD34 cliniMACS system.

The suicide moiety comprises a minimal epitope based on the epitope from CD20. Cells expressing a polypeptide comprising this sequence can be selectively killed using a lytic antibody such as Rituximab.

The combined marker and suicide polypeptide is stably expressed on the cell surface after, for example, retroviral transduction of its encoding sequence.

The marker/suicide polypeptide may have the formula:

St-R1-S1-Q-S2-R2

wherein
St is a stalk sequence which, when the polypeptide is expressed at the surface of a target cell, causes the R and Q epitopes to be projected from the cell surface;
R1 and R2 are a Rituximab-binding epitopes;
S1 and S2 are optional spacer sequences, which may be the same or different; and
Q is a QBEnd10-binding epitope.

The polypeptide may comprise the sequence shown as SEQ ID No. 30, or a variant thereof which has at least 80% identity with the sequence shown as SEQ ID No. 30 and which (i) binds QBEND10; (ii) binds Rituximab and (iii) when expressed on the surface of a cell, induces complement-mediated killing of the cell in the presence of Rituximab.

(SEQ ID No. 30)

CPYSNPSLCSGGGGSELPTQGTFSNVSTNVSPAKPTTTACPYSNPSLCSG
GGGSPAPRPPTPAPTIASQPLSLRPEACRPAAGGAVHTRGLDFACDIYIW
APLAGTCGVLLLSLVITLYCNHRNRRRVCKCPRPVV

The present invention provides a method for deleting a cell which expresses such a marker/suicide gene, which comprises the step of exposing the cells to rituximab.

Cell

The present invention relates to a cell which co-expresses a first CAR and a second CAR at the cell surface. The cell expresses a nucleic acid construct according to the first aspect of the invention.

The cell may be any eukaryotic cell capable of expressing a CAR at the cell surface, such as an immunological cell.

In particular the cell may be an immune effector cell such as a T cell or a natural killer (NK) cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+ FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The T cell of the invention may be any of the T cell types mentioned above, in particular a CTL.

Natural killer (NK) cells are a type of cytolytic cell which forms part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

CAR-expressing cells, such as CAR-expressing T or NK cells may either be created ex vivo either from a patient's own peripheral blood (1st party), or in the setting of a haematopoietic stem cell transplant from donor peripheral blood (2nd party), or peripheral blood from an unconnected donor (3rd party).

The present invention also provide a cell composition comprising CAR-expressing cells, such as CAR-expressing T and/or NK cells, according to the present invention. The cell composition may be made by transducing a blood-sample ex vivo with a nucleic acid construct according to the present invention.

Alternatively, CAR-expressing cells may be derived from ex vivo differentiation of inducible progenitor cells or embryonic progenitor cells to the relevant cell type, such as T cells. Alternatively, an immortalized cell line such as a T-cell line which retains its lytic function and could act as a therapeutic may be used.

In all these embodiments, CAR cells may be generated by introducing DNA or RNA coding for the CARs by one of many means including transduction with a viral vector, transfection with DNA or RNA.

A CAR T cell of the invention may be an ex vivo T cell from a subject. The T cell may be from a peripheral blood mononuclear cell (PBMC) sample. T cells may be activated and/or expanded prior to being transduced with CAR-encoding nucleic acid, for example by treatment with an anti-CD3 monoclonal antibody.

A CAR T cell of the invention may be made by:
(i) isolation of a T cell-containing sample from a subject or other sources listed above; and
(ii) transduction or transfection of the T cells with a nucleic acid construct encoding the first and second CAR.

The T cells may then by purified, for example, selected on the basis of co-expression of the first and second CAR.

Vector

The present invention also provides a vector which comprises a CAR-encoding nucleic acid construct as defined herein. Such a vector may be used to introduce the nucleic acid sequence(s) into a host cell so that it expresses the first and second CARs.

The vector may, for example, be a plasmid or a viral vector, such as a retroviral vector or a lentiviral vector, or a transposon based vector or synthetic mRNA.

The vector may be capable of transfecting or transducing a T cell.

Pharmaceutical Composition

The present invention also relates to a pharmaceutical composition containing a plurality of CAR-expressing cells, such as T cells or NK cells, of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The cells of the present invention may be capable of killing target cells, such as cancer cells. The target cell may be recognisable by a defined pattern of antigen expression, for example the expression of antigen A AND NOT antigen B.

The cells of the present invention may be used for the treatment of an infection, such as a viral infection.

The cells of the invention may also be used for the control of pathogenic immune responses, for example in autoimmune diseases, allergies and graft-vs-host rejection.

The cells of the invention may be used for the treatment of a cancerous disease, such as bladder cancer, breast cancer, colon cancer, endometrial cancer, kidney cancer (renal cell), leukemia, lung cancer, melanoma, non-Hodgkin lymphoma, pancreatic cancer, prostate cancer and thyroid cancer.

It is particularly suited for treatment of solid tumours where the availability of good selective single targets is limited.

The cells of the invention may be used to treat: cancers of the oral cavity and pharynx which includes cancer of the tongue, mouth and pharynx; cancers of the digestive system which includes oesophageal, gastric and colorectal cancers; cancers of the liver and biliary tree which includes hepatocellular carcinomas and cholangiocarcinomas; cancers of the respiratory system which includes bronchogenic cancers and cancers of the larynx; cancers of bone and joints which includes osteosarcoma; cancers of the skin which includes melanoma; breast cancer; cancers of the genital tract which include uterine, ovarian and cervical cancer in women, prostate and testicular cancer in men; cancers of the renal tract which include renal cell carcinoma and transitional cell carcinomas of the utterers or bladder; brain cancers including gliomas, glioblastoma multiforme and medullobastomas; cancers of the endocrine system including thyroid cancer, adrenal carcinoma and cancers associated with multiple endocrine neoplasm syndromes; lymphomas including Hodgkin's lymphoma and non-Hodgkin lymphoma; Multiple Myeloma and plasmacytomas; leukaemias both acute and chronic, myeloid or lymphoid; and cancers of other and unspecified sites including neuroblastoma.

Treatment with the cells of the invention may help prevent the escape or release of tumour cells which often occurs with standard approaches.

The invention will now be further described by way of Examples, which are meant to serve to assist one of ordinary skill in the art in carrying out the invention and are not intended in any way to limit the scope of the invention.

### EXAMPLES

### Example 1—Creation of Target Cell Populations

For the purposes of proving the principle of the invention, receptors based on anti-CD19 and anti-CD33 were arbitrarily chosen. Using retroviral vectors, CD19 and CD33 were cloned. These proteins were truncated so that they do not signal and could be stably expressed for prolonged periods. Next, these vectors were used to transduce the SupT1 cell line either singly or doubly to establish cells negative for both antigen (the wild-type), positive for either and positive for both. The expression data are shown in FIG. 3.

### Example 2—Design and Function of an AND NOT Gate

Phosphatases such as CD45 and CD148 are so potent that even a small amount entering an immunological synapse can inhibit ITAM activation. This is the basis of inhibition of the logical AND gate. Other classes of phosphatases are not as potent e.g. PTPN6 and related phosphatases. It was predicted that a small amount of PTPN6 entering a synapse by diffusion would not inhibit activation. In addition, it was predicted that if an inhibitory CAR had a sufficiently similar spacer to an activating CAR, it could co-localize within a synapse if both CARs were ligated. In this case, large amounts of the inhibitory endodomain would be sufficient to stop the ITAMS from activating when both antigens were present. In this way, an AND NOT gate could be created.

For the NOT AND gate, the second signal needs to “veto” activation. This is done by bringing an inhibitory signal into the immunological synapse, for example by bringing in the phosphatase of an enzyme such as PTPN6. We hence generated an initial AND NOT gate as follows: two CARs co-expressed whereby the first recognizes CD19, has a human CD8 stalk spacer and an activating endodomain; co-expressed with an anti-CD33 CAR with a mouse CD8 stalk spacer and an endodomain comprising of the catalytic domain of PTPN6 (FIG. 5 A with B). A suitable cassette is shown in FIG. 4 and preliminary functional data are shown in FIG. 6.

In addition, an alternative strategy was developed for generating an AND NOT gate. Immune Tyrosinase Inhibitory Motifs (ITIMs) are activated in a similar manner to ITAMS, in that they become phosphorylated by Ick upon clustering and exclusion of phosphatases. Instead of triggering activation by binding ZAP70, phosphorylated ITIMs recruit phosphatases like PTPN6 through their cognate SH2 domains. An ITIM can function as an inhibitory endodomain, as long as the spacers on the activating and inhibiting CARs can co-localize. To generate this construct, an AND NOT gate was generated as follows: two CARs co-expressed—the first recognizes CD19, has a human CD8 stalk spacer and an activating endodomain; co-expressed with an anti-CD33 CAR with a mouse CD8 stalk spacer and an ITIM containing endodomain derived from that of LAIR1 (FIG. 5 A with C).

A further, more complex AND NOT gate was also developed, whereby an ITIM is enhanced by the presence of an additional chimeric protein: an intracellular fusion of the SH2 domain of PTPN6 and the endodomain of CD148. In this design three proteins are expressed—the first recognizes CD19, has a human CD8 stalk spacer and an activating endodomain; co-expressed with an anti-CD33 CAR with a mouse CD8 stalk spacer and an ITIM containing endodomain derived from that of LAIR1. A further 2A peptide, allows co-expression of the PTPN6-CD148 fusion (FIGS. 5 A and D). It was predicted that these AND NOT gates would have a different range of inhibition: PTPN6-CD148>PTPN6>>ITIM.

T-cells were transduced with these gates and challenged with targets expressing either CD19 or CD33 alone, or both CD19 and CD33 together. All three gates responded to targets expressing only CD19, but not targets expressing both CD19 and CD33 together (FIG. 7), confirming that all three of the AND NOT gates were functional.

### Example 3: Experimental Proof of Kinetic Segregation Model of PTPN6 Based AND NOT Gate

The model of the AND NOT gate centres around the fact that the nature of the spacers used in both CARs is pivotal for the correct function of the gate. In the functional AND NOT gate with PTPN6, both CAR spacers are sufficiently similar that when both CARs are ligated, both co-localize within the synapse so the high concentration even the weak PTPN6 is sufficient to inhibit activation. If the spacers were different, segregation in the synapse will isolate the PTPN6 from the ITAM allowing activation disrupting the AND NOT gate. To test this, a control was generated replacing the murine CD8 stalk spacer with that of Fc. In this case, the test gate consisted of two CARs, the first recognizes CD19, has a human CD8 stalk spacer and an ITAM endodomain; while the second CAR recognizes CD33, has an Fc spacer and an endodomain comprising of the phosphatase from PTPN6. This gate activates in response to CD19, but also activates in response to CD19 and CD33 together (FIG. 8B, where function of this gate is compared with that of the original AND NOT, and the control AND gate). This experimental data proves the model that for a functional AND NOT gate with PTPN6, co-localizing spacers are needed.

### Example 4: Experimental Proof of Kinetic Segregation Model of ITIM Based AND NOT Gate

Similar to the PTPN6 based AND NOT gate, the ITIM based gate also requires co-localization in an immunological synapse to function as an AND NOT gate. To prove this hypothesis, a control ITIM based gate was generated as follows: two CARs co-expressed—the first recognizes CD19, has a human CD8 stalk spacer and an activating endodomain; co-expressed with an anti-CD33 CAR with an Fc spacer and an ITIM containing endodomain derived from that of LAIR1. The activity of this gate was compared with that of the original ITIM based AND NOT gate. In this case, the modified gate activated in response to targets expressing CD19, but also activated in response to cells expressing both CD19 and CD33. These data indicate that ITIM based AND NOT gates follow the kinetic segregation based model and a correct spacer must be selected to create a functional gate (FIG. 9B).

### Example 5: Summary of Model of CAR Logic Gates Generated by Kinetic Segregation

Based on current understanding of the kinetic-segregation model and the experimental data described herein, a summary of the model for a two-CAR gate is presented in FIG. 10. The Figure shows a cell expressing two CARs, each recognizing a different antigen. When either or both CARs recognize a target antigen on a cell, a synapse forms and native CD45 and CD148 are excluded from the synapse due to the bulk of their ectodomain. This sets the stage for T-cell activation. In the case that the target cell bears only one cognate antigen, the cognate CAR is ligated and the cognate CAR segregates into the synapse. The unligated CAR remains in solution on the T-cell membrane and can diffuse in and out of the synapse so that an area of high local concentration of ligated CAR with low concentration of unligated CAR forms. In this case, if the ligated CAR has an ITAM and the non-ligated CAR has ‘ligation off” type inhibitory endodomain such as that of CD148, the amount of non-ligated CAR is sufficient to inhibit activation and the gate is off. In contrast, in this case, if the ligated CAR has an ITAM and the non-ligated CAR has a ‘ligation on’ type inhibitory endodomain such as PTPN6, the amount of non-ligated CAR is insufficient to inhibit and the gate is on. When challenged by a target cell bearing both cognate antigens, both cognate CARs are ligated and form part of an immunological synapse. Importantly, if the CAR spacers are sufficiently similar, the CARs co-localize in the synapse but if the CAR spacers are sufficiently different the CARs segregate within the synapse. In this latter case, areas of membrane form whereby high concentrations of one CAR are present but the other CAR is absent. In this case since segregation is complete, even if the inhibitory endodomain is a ‘ligation off’ type, the gate is on. In the former case, areas of membrane form with high concentrations of both CARs mixed together. In this case, since both endodomains are concentrated, even if the inhibitory endodomain is ‘ligation on’ type, the gate is off. By selecting the correct combination of spacer and endodomain logic can be programmed into a CAR T-cell.

### Example 6: Testing the AND NOT Gate with Extended Spacers

To test if the ANDNOT gate could function on extended spacer lengths, both the activating CAR (anti-CD19) and the inhibiting CAR (anti-CD33) spacers were substituted for longer spacers. The Fc region of human IgM and IgG were used to extend the spacer length. The Fc of IgM contains and additional Ig domain compared to IgG, for this reason the IgM spacer was placed on the anti-CD19 CAR which is known to have a membrane proximal binding epitope. In contrast the anti-CD33 binding epitope is located on a distal end of the molecule, thus the relatively shorter IgG spacer was used on this CAR (see FIG. 30). The extended spacer ANDNOT gate construct was transduced into a mouse T-cell line. Then a fixed number of transduced T-cells were co-cultured with a varying number of target cells for 16-24 hours, after which the amount of IL-2 secreted in the supernatant was analysed via ELISA.

The results are shown in FIG. 11. The AND NOT gate worked well with the IgG/IgM spacer pair.

### Example 7: Testing the Robustness of the ANDNOT Gate Platform

To test the robustness of the ANDNOT gate platform, the binding domain from the inhibitory CAR (anti-CD33) was substituted with two other unrelated binders (anti-GD2 and anti-EGFRvIII). The scFv fragment for anti-GD2 or anti-EGFRvIII was substituted for anti-CD33 on the inhibitory CAR in the ANDNOT gate platform with either a truncated SHP-1 or LAIR cytosolic domain. These constructs were transduced into a mouse T-cell line and a fixed number of T-cells were co-cultured with a varying number of target cells. After 16-24 hours of co-culture the amount of IL-2 secreted in the supernatant was analysed via ELISA.

The results are shown in FIG. 12. The AND NOT gate worked well with the anti-CD19/anti-GD2 binders and the anti-CD19/anti-EGFRvIII binders with either the truncated SHP-1 or LAIR cytosolic domains.

### Example 8—Dissection of TYRP1 Lysozomal Retention Signals

The ability of the Tyrosinase-related protein 1 (TYRP1) retention signal to cause retention of a polypeptide when in the context of a more complex endodomain was determined using a number of constructs (FIG. 16). The wild-type construct was compared with constructs where enhanced Green Fluorescent Protein (eGFP) was added or replaced the TYRP1 endodomain. Where eGFP was added, it was placed either after or before the native endodomain so the retention signal was either in its native location (just under the membrane), or distal to it.

All constructs are co-expressed with IRES.CD34. Staining of transduced SupT1 cells is shown with intracellular and surface staining in FIG. 16.

It was found that replacement of the endodomain resulted in very bright surface expression, introduction of eGFP after the retention signal to almost no surface expression and introduction before the retention signal to intermediate surface expression (FIG. 16).

### Example 9—Modulation of the Relative Expression of a Transmembrane Protein Co-Expressed from a Single Expression Cassette with a Separate Protein

An expression cassette encoding two CAR transmembrane proteins was modified such that one of the CAR proteins had the lysozomal retention signal from TYRP1 introduced either proximal or distal to the membrane. Expression of each of these two new variants at the cell surface was compared with that of the original unmodified CAR protein.

PBMCs were isolated from blood and stimulated using PHA and IL-2. Two days later the cells were transduced on retronectin coated plates with retro virus containing the CD19:CD33 CAR construct. On day 5 the expression level of the two CARs translated by the construct was evaluated via flow cytometry and the cells were depleted of CD56+ cells (predominantly NK cells). On day 6 the PBMCs were placed in a co-culture with target cells at a 1:2 effector to target cell ratio. On day 8 the supernatant was collected and analysed for IFN-gamma secretion via ELISA.

The pattern observed with Tyrp1-eGFP fusions was observed with some reduction of expression of modified transmembrane protein with the distal retention signal and marked reduction in the case of proximal retention signal. As expected, expression of the second transmembrane protein from the cassette was not altered (FIG. 17).

### Example 10—Modulation of Expression Using a Retention Signal from the Adenoviral E3/19K Protein

The human adenovirus E3/19K protein is a type I transmembrane glycoprotein of the Endoplasmic Reticulum/Golgi that abrogates cell surface transport of major histocompatibility complex class I (MHC-I) and MHC-I-related chain A and B (MICA/B) molecules. The retention motif was identified to be depended on the cytosolic tail of the adenovirus E3/19K protein. More specifically, the last 6aa DEKKMP was found to be the most important for retention. The optimal positioning was found to be at the c-terminus of the protein.

An expression cassette encoding two CAR transmembrane proteins, as described in Example 7, was modified such that one of the CAR proteins had the retention motif from adenovirus E3/19K protein. In this experiment, the retention motif on the second CAR in the expression cassette (the anti-CD33 inhibitory CAR).

Constructs were generated comprising either the entire cytosolic tail of adenovirus E3/19K protein or only the last 6aa from E3/19K (DEKKMP), which were found to be critical for its Golgi/ER retention ability (FIG. 18). These constructs were transfected into 293T cells and stained primarily with a chimeric soluble CD19-Rabbit Fc and a chimeric soluble CD33-Mouse Fc proteins. These cells were then subsequently stained with an anti-Rabbit Fc-FITC and an anti-Mouse Fc-APC (FIG. 19). These cells show a clear retention when the full length adenovirus E3/19K protein, or the DEKKMP motif, was placed on the anti-CD33 receptor but had no effect on anti-CD19 receptor expression levels.

### Example 11—Modulation of Expression by Altering the Signal Peptide—Swapping in the Murine Ig Kappa Chain V-III Signal Sequence

PCT/GB2014/053452 describes a vector system encoding two chimeric antigen receptors (CARs), one against CD19 and one against CD33. The signal peptide used for the CARs in that study was the signal peptide from the human CD8a signal sequence. For the purposes of this study, this was substituted with the signal peptide from the murine Ig kappa chain V-III region, which has the sequence: METDTLILWVLLLLVPGSTG (hydrophobic residues hightlited in bold). In order to establish that the murine Ig kappa chain V-III signal sequence functioned as well as the signal sequence from human CD8a, a comparative study was performed. For both signal sequences, functional expression of the anti-CD33 CAR and the anti-CD19 CAR was observed. This substituted signal sequence and all subsequent mutations thereof were transiently transfected into 293T cells. Three days after transfection the 293T cells were stained with both soluble chimeric CD19 fused with rabbit Fc chain and soluble chimeric CD33 fused with mouse Fc chain. All cells were then stained with anti-Rabbit Fc-FITC and anti-mouse Fc-APC. Flow cytometry plots show the substituted signal sequence as a comparison with non-transfected (NT) and the construct with Cd8 signal sequences (FIG. 22). The murine Ig kappa chain V-III signal sequence was found to function as well as the signal sequence from human CD8a.

### Example 12—Altering Relative Expression by Deleting Hydrophobic Residues in the Signal Peptide

Hydrophobic residues were deleted in a stepwise fashion and the effect on the relative expression of the anti-CD33 CAR and the anti-CD19 CAR was observed. The effect of one, two, three and four amino acid deletions was investigated and the results are shown in FIGS. 23 to 26 respectively.

All mutant constructs showed a decrease in relative expression of the anti-CD19 CAR compared to the anti-CD33 CAR. The relative decrease of anti-CD19 CAR expression was greater with a greater number of amino acid deletions from 1 to 3, but then plateaued out (four deletions gave a similar decrease in expression as three deletions).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cell biology or related fields are intended to be within the scope of the following claims.

1. A nucleic acid construct comprising the following structure:
A-X-B
in which
X is a nucleic acid sequence which encodes a cleavage site, and
A and B are nucleic acid sequences encoding a first and a second chimeric antigen receptor (CAR), each CAR comprising:
(i) an antigen-binding domain,
(ii) a spacer,
(iii) a trans-membrane domain, and
(iv) an endodomain
wherein the antigen binding domains of the first and second CARs bind to different antigens, wherein one of the first or second CARs is an activating CAR comprising an activating endodomain and the other CAR is an inhibitory CAR comprising a ligation-on inhibitory endodomain, and wherein:
(a) the first and/or second CAR comprises an intracellular retention signal and/or
(b) the signal peptide of the first or second CAR comprises one or more mutation(s) such that it has fewer hydrophobic amino acids.

2. The nucleic acid construct according to claim 1, wherein the spacers of the first and second CARs are sufficiently different as to prevent cross-pairing, but to be sufficiently similar to cause the CARs to co-localise at the T cell membrane.

3-4. (canceled)

5. The nucleic acid construct according to claim 1, wherein one of the first or second CARs in an activating CAR comprising an activating endodomain and the other CAR is an inhibitory CAR comprising a ligation-on inhibitory endodomain, which inhibitory CAR does not significantly inhibit T-cell activation by the activating CAR in the absence of inhibitory CAR ligation but inhibits T-cell activation by the activating CAR when the inhibitory CAR is ligated.

6. The nucleic acid construct according to claim 5, wherein the ligation-on inhibitory endodomain comprises at least part of a phosphatase.

7. The nucleic acid construct according to claim 6, wherein the ligation-on inhibitory endodomain comprises all or part of PTPN6.

8-13. (canceled)

14. The nucleic acid construct according to claim 1, wherein the first and/or second CAR comprises an intracellular retention signal which directs the CAR to a lysozomal, endosomal or Golgi compartment.

15. The nucleic acid construct according to claim 1, wherein the first and/or second CAR comprises an intracellular retention signal selected from the following group: an endocytosis signal; a Golgi retention signal; a trans-Golgi network (TGN) recycling signal; an endoplasmic reticulum (ER) retention signal; and a lysosomal sorting signal.

16-22. (canceled)

23. The nucleic acid construct according to claim 1, wherein the signal peptide of the first or second CAR comprises one or more mutation(s) such that it has fewer hydrophobic amino acids than a) its wild-type sequence or b) the signal peptide of the other CAR.

24. The nucleic acid construct according to claim 23, wherein the signal peptide of the activating CAR comprises one or more mutation(s) such that it has fewer hydrophobic amino acids, such that when the nucleic acid construct is expressed in a cell, the relative expression level of the activating CAR at the cell surface is reduced in comparison with the inhibitory CAR.

25. The nucleic acid construct according to claim 23, wherein the hydrophobic amino acid(s) removed or replaced by mutation is/are selected from: Alanine (A), Valine (V), Isoleucine Leucine (L), Methionine (M), Phenylalanine (P), Tyrosine (Y), and Tryptophan (W).

26. The nucleic acid construct according to claim 23, wherein the hydrophobic amino acid(s) removed or replaced by mutation is/are selected from: Valine (V), Isoleucine (I), Leucine (L), and Tryptophan (W).

27. The nucleic acid construct according to claim 1, wherein the signal peptide of the first and second CAR differ in their number of hydrophobic amino acids and wherein one signal peptide comprises up to five more hydrophobic amino acids than the other signal peptide.

28. A vector comprising a nucleic acid construct according to claim 1.

29. (canceled)

30. A cell comprising a nucleic acid construct according to claim 1.

31. (canceled)

32. A method for making a cell according to claim 30, which comprises the step of introducing into a cell: a nucleic acid construct comprising the following structure:
A-X-B
in which
X is a nucleic acid sequence which encodes a cleavage site, and
A and B are nucleic acid sequences encoding a first and a second chimeric antigen receptor (CAR), each CAR comprising:
(i) an antigen-binding domain,
(ii) a spacer,
(iii) a trans-membrane domain, and
(iv) an endodomain
wherein the antigen binding domains of the first and second CARs bind to different antigens, wherein one of the first or second CARs is an activating CAR comprising an activating endodomain and the other CAR is an inhibitory CAR comprising a ligation-on inhibitory endodomain, and wherein:
(a) the first and/or second CAR comprises an intracellular retention signal and/or
(b) the signal peptide of the first or second CAR comprises one or more mutation(s) such that it has fewer hydrophobic amino acids.

33. The method according to claim 32, wherein the cell is from a sample isolated from a subject.

34. A pharmaceutical composition comprising a plurality of cells according to claim 30.

35. A method for treating and/or preventing a disease, which comprises the step of administering a pharmaceutical composition according to claim 32 to a subject.

36. The method according to claim 35, which comprises the following steps:
(i) isolating a T and/or NK cell-containing sample from a subject;
(ii) transducing or transfecting the T and/or NK cells with: a nucleic acid construct comprising the following structure:
A-X-B
in which
X is a nucleic acid sequence which encodes a cleavage site, and
A and B are nucleic acid sequences encoding a first and a second chimeric antigen receptor (CAR), each CAR comprising:
(1) an antigen-binding domain,
(2) a spacer,
(3) a trans-membrane domain, and
(4) an endodomain
wherein the antigen binding domains of the first and second CARs bind to different antigens, wherein one of the first or second CARs is an activating CAR comprising an activating endodomain and the other CAR is an inhibitory CAR comprising a ligation-on inhibitory endodomain, and wherein:
(a) the first and/or second CAR comprises an intracellular retention signal and/or
(b) the signal peptide of the first or second CAR comprises one or more mutation(s) such that it has fewer hydrophobic amino acids; and
(iii) administering the T and/or NK cells from (ii) to a the subject.

37. The method according to claim 35, wherein the disease is a cancer.

38-39. (canceled)


SEQUENCE LISTING (255 sequences)

SEQ ID NO: 1 - protein, 7 aa, Artificial Sequence
  Tyrosinase-related protein (TYRP)-1 intracellular retention signal
NQPLLTD

SEQ ID NO: 2 - protein, 15 aa, Artificial Sequence
  Adenoviral E3/19K intracellular retention signal
KYKSRRSFIDEKKMP

SEQ ID NO: 3 - protein, 6 aa, Artificial Sequence
  Adenoviral E3/19K intracellular retention signal
DEKKMP

SEQ ID NO: 4 - protein, 1114 aa, Artificial Sequence
  CAR AND NOT gate which recognizes CD19 AND NOT CD33 based on PTPN6 phosphatase
MSLPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMAVPTQVLGLLLLWLTDARCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSMDPATTTKPVLRTPSPVHPTGTSQPQRPEDCRPRGSVKGTGLDFACDIYWAPLAGICVALLLSLIITLICYHRSRKRVCKSGGGSFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKL

SEQ ID NO: 5 - protein, 918 aa, Artificial Sequence
  CAR AND NOT gate which recognizes CD19 AND NOT CD33 and is based on an ITIM containing endodomain from LAIR1
MSLPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMAVPTQVLGLLLLWLTDARCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSMDPATTTKPVLRTPSPVHPTGTSQPQRPEDCRPRGSVKGTGLDFACDILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 6 - protein, 1363 aa, Artificial Sequence
  CAR AND NOT gate which recognizes CD19 AND NOT CD33 and recruits a PTPN6-CD148 fusion protein to an ITIM containing endodomain
MSLPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDPTTTPAPRPPTPAPTIASQPLSLRPEACRPAAGGAVHTRGLDFACDIFWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMAVPTQVLGLLLLWLTDARCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSMDPATTTKPVLRTPSPVHPTGTSQPQRPEDCRPRGSVKGTGLDFACDILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARHRAEGRGSLLTCGDVEENPGPWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYSGGGGSFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIASGS

SEQ ID NO: 7 - protein, 36 aa, Artificial Sequence
  nucleic acid construct comprising a tyrp-1 retention protein sequence
RARRSMDEANQPLLTDQYQCYAEEYEKLQNPNQSVV

SEQ ID NO: 8 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 9 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 10 - protein, 20 aa, Artificial Sequence
  spacer sequence, human IgG1 hinge
AEPKSPDKTHTCPPCPKDPK

SEQ ID NO: 11 - protein, 185 aa, Artificial Sequence
  spacer sequence, CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 12 - protein, 259 aa, Artificial Sequence
  spacer sequence, CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 13 - protein, 140 aa, Artificial Sequence
  comprising CD28 transmembrane domain and CD3 Z endodomain
FWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 180 aa, Artificial Sequence
  comprising CD28 transmembrane domain and CD28 and CD3 Zeta endodomains
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 216 aa, Artificial Sequence
  comprising CD28 transmembrane domain and CD28, OX40 and CD3 Zeta endodomains
FWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 595 aa, Artificial Sequence
  sequence of PTPN6
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYYATRVNAADIENRVLELNKKQESEDTAKAGFWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKLEVLQSQKGQESEYGNITYPPAMKNAHAKASRTSSKHKEDVYENLHTKNKREEKVKKQRSADKEKSKGSLKRK

SEQ ID NO: 17 - protein, 272 aa, Artificial Sequence
  sequence of phosphatase domain of PTPN6
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 18 - protein, 97 aa, Artificial Sequence
  PDCD1 endodomain
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 19 - protein, 141 aa, Artificial Sequence
  BTLA4 endodomain
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 20 - protein, 168 aa, Artificial Sequence
  LILRB1 endodomain
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 21 - protein, 101 aa, Artificial Sequence
  LAIR1 endodomain
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 22 - protein, 62 aa, Artificial Sequence
  CTLA4 endodomain
FLLWILAAVSSGLFFYSFLLTAVSLSKMLKKRSPLTTGVYVKMPPTEPECEKQFQPYFIPIN

SEQ ID NO: 23 - protein, 111 aa, Artificial Sequence
  KIR2DL1 endodomain
GNSRHLHVLIGTSVVIIPFAILLFFLLHRWCANKKNAVVMDQEPAGNRTVNREDSDEQDPQEVTYTQLNHCVFTQRKITRPSQRPKTPPTDIIVYTELPNAESRSKVVSCP

SEQ ID NO: 24 - protein, 143 aa, Artificial Sequence
  KIR2DL4 endodomain
GIARHLHAVIRYSVAIILFTILPFFLLHRWCSKKKENAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 25 - protein, 143 aa, Artificial Sequence
  KIR2DL5 endodomain
TGIRRHLHILIGTSVAIILFIILFFFLLHCCCSNKKNAAVMDQEPAGDRTVNREDSDDQDPQEVTYAQLDHCVFTQTKITSPSQRPKTPPTDTTMYMELPNAKPRSLSPAHKHHSQALRGSSRETTALSQNRVASSHVPAAGI

SEQ ID NO: 26 - protein, 111 aa, Artificial Sequence
  KIR3DL1 endodomain
KDPRHLHILIGTSVVIILFILLLFFLLHLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 27 - protein, 97 aa, Artificial Sequence
  KIR3DL3 endodomain
KDPGNSRHLHVLIGTSVVIIPFAILLFFLLHRWCANKKNAVVMDQEPAGNRTVNREDSDEQDPQEVTYAQLNHCVFTQRKITRPSQRPKTPPTDTSV

SEQ ID NO: 28 - protein, 807 aa, Artificial Sequence [sequence omitted]

SEQ ID NO: 29 - protein, 434 aa, Artificial Sequence
  PTPN6-CD148 fusion protein
ETLLQAKGEPWTFLVRESLSQPGDFVLSVLSDQPKAGPGSPLRVTHIKVMCEGGRYTVGGLETFDSLTDLVEHFKKTGIEEASGAFVYLRQPYRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 30 - protein, 136 aa, Artificial Sequence
  marker/suicide polypeptide
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 31 - protein, 19 aa, Artificial Sequence
  cleavage sequence
LLNFDLLKLAGDVESNPGG

SEQ ID NO: 32 - protein, 20 aa, Artificial Sequence
  2A-like sequence
YHADYYKQRLIHDVEMNPGP

SEQ ID NO: 33 - protein, 20 aa, Artificial Sequence
  2A-like sequence
HYAGYFADLLIHDIETNPGP

SEQ ID NO: 34 - protein, 20 aa, Artificial Sequence
  2A-like sequence
QCTNYALLKLAGDVESNPGP

SEQ ID NO: 35 - protein, 19 aa, Artificial Sequence
  2A-like sequence
ATNFSLLKQAGDVEENPGP

SEQ ID NO: 36 - protein, 19 aa, Artificial Sequence
  2A-like sequence
AARQMLLLLSGDVETNPGP

SEQ ID NO: 37 - protein, 20 aa, Artificial Sequence
  2A-like sequence
RAEGRGSLLTCGDVEENPGP

SEQ ID NO: 38 - protein, 20 aa, Artificial Sequence
  2A-like sequence
TRAEIEDELIRAGIESNPGP

SEQ ID NO: 39 - protein, 20 aa, Artificial Sequence
  2A-like sequence
TRAEIEDELIRADIESNPGP

SEQ ID NO: 40 - protein, 20 aa, Artificial Sequence
  2A-like sequence
AKFQIDKILISGDVELNPGP

SEQ ID NO: 41 - protein, 20 aa, Artificial Sequence
  2A-like sequence
SSIIRTKMLVSGDVEENPGP

SEQ ID NO: 42 - protein, 20 aa, Artificial Sequence
  2A-like sequence
CDAQRQKLLLSGDIEQNPGP

SEQ ID NO: 43 - protein, 20 aa, Artificial Sequence
  2A-like sequence
YPIDFGGFLVKADSEFNPGP

SEQ ID NO: 44 - protein, 57 aa, Artificial Sequence
  cleavage sequence
VTELLYRMKRAETYCPRPLAIHPTEARHKQKIVAPVKQTLNFDLLKLAGDVESNPGP

SEQ ID NO: 45 - protein, 40 aa, Artificial Sequence
  cleavage sequence
LLAIHPTEARHKQKIVAPVKQTLNFDLLKLAGDVESNPGP

SEQ ID NO: 46 - protein, 33 aa, Artificial Sequence
  cleavage sequence
EARHKQKIVAPVKQTLNFDLLKLAGDVESNPGP

SEQ ID NO: 47 - protein, 24 aa, Artificial Sequence
  cleavage sequence
APVKQTLNFDLLKLAGDVESNPGP

SEQ ID NO: 48 - protein, 5 aa, Artificial Sequence
  linker
SGGGS

SEQ ID NO: 49 - protein, 4 aa, Artificial Sequence
  intracellular retention signal
  misc_feature (4)..(4): Xaa can be any naturally occurring amino acid
NPFX

SEQ ID NO: 50 - protein, 4 aa, Artificial Sequence
  intracellular retention signal
KDEL

SEQ ID NO: 51 - protein, 4 aa, Artificial Sequence
  intracellular retention signal
  misc_feature (3)..(4): Xaa can be any naturally occurring amino acid
KKXX

SEQ ID NO: 52 - protein, 5 aa, Artificial Sequence
  intracellular retention signal
  misc_feature (2)..(2): Xaa can be any naturally occurring amino acid
  misc_feature (4)..(5): Xaa can be any naturally occurring amino acid
KXKXX

SEQ ID NO: 53 - protein, 4 aa, Artificial Sequence
  intracellular retention signal
  misc_feature (3)..(3): Xaa can be any naturally occurring amino acid
NPXY

SEQ ID NO: 54 - protein, 4 aa, Artificial Sequence
  intracellular retention signal
  misc_feature (2)..(3): Xaa can be any naturally occurring amino acid
  MISC_FEATURE (4)..(4): Xaa is an amino acid with a bulky hydrophobic side chain
YXXX

SEQ ID NO: 55 - protein, 8 aa, Artificial Sequence
  intracellular retention signal
  misc_feature (3)..(5): Xaa can be any naturally occurring amino acid
DEXXXLLI

SEQ ID NO: 56 - protein, 5 aa, Artificial Sequence
  intracellular retention signal
  misc_feature (2)..(3): Xaa can be any naturally occurring amino acid
DXXLL

SEQ ID NO: 57 - protein, 4 aa, Artificial Sequence
  intracellular retention signal
DPFW

SEQ ID NO: 58 - protein, 5 aa, Artificial Sequence
  intracellular retention signal
  misc_feature (2)..(2): Xaa can be any naturally occurring amino acid
  misc_feature (4)..(4): Xaa can be any naturally occurring amino acid
FXDXF

SEQ ID NO: 59 - protein, 6 aa, Artificial Sequence
  intracellular retention signal
  MISC_FEATURE (2)..(2): Xaa is an amino acid with a bulky hydrophobic side chain
  misc_feature (3)..(3): Xaa can be any naturally occurring amino acid
  MISC_FEATURE (4)..(4): Xaa is an amino acid with a bulky hydrophobic side chain
LXXXDE

SEQ ID NO: 60 - protein, 5 aa, Artificial Sequence
  intracellular retention signal
LLDLL

SEQ ID NO: 61 - protein, 5 aa, Artificial Sequence
  intracellular retention signal
PWDLW

SEQ ID NO: 62 - protein, 20 aa, Mus musculus
METDTLILWVLLLLVPGSTG

SEQ ID NO: 63 - protein, 21 aa, Artificial Sequence
  signal peptide derived from Human CD8a
MSLPVTALLLPLALLLHAARP

SEQ ID NO: 64 - protein, 248 aa, Artificial Sequence
  scFv aCD19
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 65 - protein, 47 aa, Homo sapiens
PTTTPAPRPPTPAPTIASQPLSLRPEACRPAAGGAVHTRGLDFACDI

SEQ ID NO: 66 - protein, 27 aa, Homo sapiens
FWVLVVVGGVLACYSLLVTVAFIIFWV

SEQ ID NO: 67 - protein, 113 aa, Homo sapiens
RRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 20 aa, Artificial Sequence
  2A peptide
RAEGRGSLLTCGDVEENPGP

SEQ ID NO: 69 - protein, 18 aa, Artificial Sequence
  signal peptide derived from mouse Ig kappa
MAVPTQVLGLLLLWLTDA

SEQ ID NO: 70 - protein, 254 aa, Artificial Sequence
  scFv aCD33
RCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSM

SEQ ID NO: 71 - protein, 43 aa, Mus musculus
TTTKPVLRTPSPVHPTGTSQPQRPEDCRPRGSVKGTGLDFACD

SEQ ID NO: 72 - protein, 22 aa, Mus musculus
IYWAPLAGICVALLLSLIITLI

SEQ ID NO: 73 - protein, 11 aa, Artificial Sequence
  truncated mouse CD8a intracellular domain
CYHRSRKRVCK

SEQ ID NO: 74 - protein, 280 aa, Artificial Sequence
  truncated human SHP-1
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQFIETTKKKL

SEQ ID NO: 75 - protein, 21 aa, Homo sapiens
ILIGVSVVFLFCLLLLVLFCL

SEQ ID NO: 76 - protein, 4 aa, Mus musculus
METD

SEQ ID NO: 77 - protein, 11 aa, Mus musculus
TLILWVLLLLV

SEQ ID NO: 78 - protein, 5 aa, Mus musculus
PGSTG

SEQ ID NO: 79 - protein, 25 aa, Homo sapiens
MNRGVPFRHLLLVLQLALLPAATQG

SEQ ID NO: 80 - protein, 23 aa, Homo sapiens
MYLWLKLLAFGFAFLDTEVFVTG

SEQ ID NO: 81 - protein, 21 aa, Homo sapiens
MALPVTALLLPLALLLHAARP

SEQ ID NO: 82 - protein, 21 aa, Homo sapiens
MRPRLWLLLAAQLTVLHGNSV

SEQ ID NO: 83 - protein, 24 aa, Homo sapiens
MSFPCKFVASFLLIFNVSSKGAVS

SEQ ID NO: 84 - protein, 24 aa, Homo sapiens
MPMGSLQPLATLYLLGMLVASCLG

SEQ ID NO: 85 - protein, 25 aa, Homo sapiens
MAGPPRLLLLPLLLALARGLPGALA

SEQ ID NO: 86 - protein, 24 aa, Homo sapiens
MSAPKLLSLGCIFFPLLLFQQARA

SEQ ID NO: 87 - protein, 19 aa, Homo sapiens
MRFFVPLFLVGILFPAILA

SEQ ID NO: 88 - protein, 23 aa, Homo sapiens
MPGFLVRILPLLLVLLLLGPTRG

SEQ ID NO: 89 - protein, 24 aa, Homo sapiens
MKRFLFLLLTISLLVMVQIQTGLS

SEQ ID NO: 90 - protein, 21 aa, Homo sapiens
MQGPPLLTAAHLLCVCTAALA

SEQ ID NO: 91 - protein, 22 aa, Homo sapiens
MGPTSGPSLLLLLLTHLPLALG

SEQ ID NO: 92 - protein, 18 aa, Homo sapiens
MQPILLLLAFLLLPRADA

SEQ ID NO: 93 - protein, 20 aa, Homo sapiens
MEAPAQLLFLLLLWLPDTTR

SEQ ID NO: 94 - protein, 20 aa, Homo sapiens
MVLQTQVFISLLLWISGAYG

SEQ ID NO: 95 - protein, 19 aa, Homo sapiens
MAWAPLLLTLLAHCTDCWA

SEQ ID NO: 96 - protein, 22 aa, Homo sapiens
MLKPSLPFTSLLFLQLPLLGVG

SEQ ID NO: 97 - protein, 20 aa, Homo sapiens
MAWASRLGLLLALLLPVVGA

SEQ ID NO: 98 - protein, 23 aa, Homo sapiens
MGVPRPQPWALGLLLFLLPGSLG

SEQ ID NO: 99 - protein, 4 aa, Artificial Sequence
  ITAM motif
  misc_feature (2)..(3): Xaa can be any naturally occurring amino acid
  MISC_FEATURE (4)..(4): Xaa may be Leu or Ile
YXXX

SEQ ID NO: 100 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 101 - protein, 7 aa, Artificial Sequence
  consensus Tobacco Etch Virus (TEV) cleavage site
ENLYFQS

SEQ ID NO: 102 - protein, 8 aa, Artificial Sequence
  motif involved in cleavage activity
  misc_feature (2)..(2): Xaa can be any naturally occurring amino acid
  misc_feature (4)..(4): Xaa can be any naturally occurring amino acid
DXEXNPGP

SEQ ID NO: 103 - protein, 8 aa, Artificial Sequence
  cleavage site
  MISC_FEATURE (2)..(2): Xaa may be Ile, Val, Met or Ser
  MISC_FEATURE (4)..(4): Xaa may be Thr, Met, Ser, Leu, Glu, Gln or Phe
DXEXNPGP

SEQ ID NO: 104 - protein, 4 aa, Artificial Sequence
  C-terminal signal, motif of postassium channels
  misc_feature (2)..(2): Xaa can be any naturally occurring amino acid
RXRR

SEQ ID NO: 105 - protein, 12 aa, Homo sapiens
INFDNPVYQKTT

SEQ ID NO: 106 - protein, 12 aa, Homo sapiens
VEIGNPTYKMYE

SEQ ID NO: 107 - protein, 12 aa, Homo sapiens
TNFTNPVYATLY

SEQ ID NO: 108 - protein, 12 aa, Drosophila sp.
GNFANPVYESMY

SEQ ID NO: 109 - protein, 12 aa, Caenorhabditis elegans
TTFTNPVYELED

SEQ ID NO: 110 - protein, 12 aa, Caenorhabditis elegans
LRVDNPLYDPDS

SEQ ID NO: 111 - protein, 12 aa, Homo sapiens
IIFENPMYSARD

SEQ ID NO: 112 - protein, 12 aa, Homo sapiens
TNFENPIYAQME

SEQ ID NO: 113 - protein, 12 aa, Homo sapiens
DTGENPIYKSAV

SEQ ID NO: 114 - protein, 11 aa, Homo sapiens
TTVVNPKYEGK

SEQ ID NO: 115 - protein, 12 aa, Drosophila sp.
WDTENPIYKQAT

SEQ ID NO: 116 - protein, 11 aa, Drosophila sp.
STFKNPMYAGK

SEQ ID NO: 117 - protein, 12 aa, Homo sapiens
HGYENPTYRFLE

SEQ ID NO: 118 - protein, 12 aa, Homo sapiens
NGYENPTYKFFE

SEQ ID NO: 119 - protein, 12 aa, Drosophila sp.
NGYENPTYKYFE

SEQ ID NO: 120 - protein, 12 aa, Homo sapiens
YASSNPEYLSAS

SEQ ID NO: 121 - protein, 12 aa, Homo sapiens
GSVQNPVYHNQP

SEQ ID NO: 122 - protein, 12 aa, Homo sapiens
TAVGNPEYLNTV

SEQ ID NO: 123 - protein, 12 aa, Homo sapiens
ISLDNPDYQQDF

SEQ ID NO: 124 - protein, 11 aa, Homo sapiens
RKRSHAGYQTI

SEQ ID NO: 125 - protein, 10 aa, Homo sapiens
KHHHAGYEQF

SEQ ID NO: 126 - protein, 11 aa, Gallus gallus
KKHHNTGYEQF

SEQ ID NO: 127 - protein, 11 aa, Gallus gallus
RRKSRTGYQSV

SEQ ID NO: 128 - protein, 11 aa, Gallus gallus
RRKSYAGYQTL

SEQ ID NO: 129 - protein, 12 aa, Drosophila sp.
RRRSTSRGYMSF

SEQ ID NO: 130 - protein, 11 aa, Lumbricus terrestris
RKRSRRGYESV

SEQ ID NO: 131 - protein, 10 aa, Homo sapiens
KSIRSGYEVM

SEQ ID NO: 132 - protein, 12 aa, Homo sapiens
HCGGPRPGYETL

SEQ ID NO: 133 - protein, 12 aa, Mus musculus
HCRTRRAEYETL

SEQ ID NO: 134 - protein, 10 aa, Homo sapiens
RRRPSAYQAL

SEQ ID NO: 135 - protein, 9 aa, Homo sapiens
RRRSYQNIP

SEQ ID NO: 136 - protein, 10 aa, Homo sapiens
KKHCSYQDIL

SEQ ID NO: 137 - protein, 10 aa, Mus musculus
RRRSAYQDIR

SEQ ID NO: 138 - protein, 11 aa, Rattus norvegicus
RKRRRSYQDIM

SEQ ID NO: 139 - protein, 13 aa, Rattus norvegicus
KFCKSKERNYHTL

SEQ ID NO: 140 - protein, 14 aa, Drosophila sp.
KFYKARNERNYHTL

SEQ ID NO: 141 - protein, 14 aa, Homo sapiens
KIRLRCQSSGYQRI

SEQ ID NO: 142 - protein, 14 aa, Mus musculus
KIRQRHQSSAYQRI

SEQ ID NO: 143 - protein, 15 aa, Homo sapiens
HFCLYRKRPGYDQLN

SEQ ID NO: 144 - protein, 11 aa, Homo sapiens
SLSRGSGYKEI

SEQ ID NO: 145 - protein, 13 aa, Homo sapiens
RRLRKGYTPLMET

SEQ ID NO: 146 - protein, 14 aa, Homo sapiens
RRAGHSSYTPLPGS

SEQ ID NO: 147 - protein, 18 aa, Dictyostelium sp.
KKLRQQKQQGYQAIINNE

SEQ ID NO: 148 - protein, 15 aa, Dictyostelium sp.
RSKSNQNQSYNLIQL

SEQ ID NO: 149 - protein, 17 aa, Dictyostelium sp.
RKTFYNNNQYNGYNIIN

SEQ ID NO: 150 - protein, 10 aa, Homo sapiens
PLSYTRFSLA

SEQ ID NO: 151 - protein, 11 aa, Homo sapiens
MTKEYQDLQHL

SEQ ID NO: 152 - protein, 10 aa, Homo sapiens
SYKYSKVNKE

SEQ ID NO: 153 - protein, 10 aa, Homo sapiens
PAAYRGVGDD

SEQ ID NO: 154 - protein, 10 aa, Homo sapiens
TGVYVKMPPT

SEQ ID NO: 155 - protein, 10 aa, Homo sapiens
LISYKGLPPE

SEQ ID NO: 156 - protein, 11 aa, Rattus norvegicus
ASDYQRLNLKL

SEQ ID NO: 157 - protein, 11 aa, Human immunodeficiency virus type 1
RQGYSPLSFQT

SEQ ID NO: 158 - protein, 19 aa, Homo sapiens
RMQAQPPGYRHVADGEDHA

SEQ ID NO: 159 - protein, 7 aa, Artificial Sequence
  Dileucine-based sorting signal
  MISC_FEATURE (1)..(1): Xaa may be Asp or Glu
  misc_feature (2)..(4): Xaa can be any naturally occurring amino acid
  MISC_FEATURE (7)..(7): Xaa may be Leu or Ile
XXXXLLX

SEQ ID NO: 160 - protein, 9 aa, Homo sapiens
SDKQTLLPN

SEQ ID NO: 161 - protein, 9 aa, Rattus norvegicus
DERAPLIRT

SEQ ID NO: 162 - protein, 9 aa, Homo sapiens
QEKDPLLKN

SEQ ID NO: 163 - protein, 9 aa, Coturnix sp.
TERNPLLKS

SEQ ID NO: 164 - protein, 9 aa, Homo sapiens
GENSPLLSG

SEQ ID NO: 165 - protein, 9 aa, Homo sapiens
EEKQPLLME

SEQ ID NO: 166 - protein, 9 aa, Oryzias latipes
GERQPLLQS

SEQ ID NO: 167 - protein, 9 aa, Gallus gallus
PEIQPLLTE

SEQ ID NO: 168 - protein, 9 aa, Carassius auratus
EGRQPLLGD

SEQ ID NO: 169 - protein, 9 aa, Homo sapiens
EANQPLLTD

SEQ ID NO: 170 - protein, 9 aa, Gallus gallus
ELHQPLLTD

SEQ ID NO: 171 - protein, 9 aa, Danio rerio
REFEPLLNA

SEQ ID NO: 172 - protein, 9 aa, Homo sapiens
EEKMAILMD

SEQ ID NO: 173 - protein, 9 aa, Homo sapiens
EEKLAILSQ

SEQ ID NO: 174 - protein, 9 aa, Mus musculus
SERDVLLDE

SEQ ID NO: 175 - protein, 9 aa, Homo sapiens
SERRNLLED

SEQ ID NO: 176 - protein, 9 aa, Rattus norvegicus
DDSGDLLPG

SEQ ID NO: 177 - protein, 9 aa, Homo sapiens
SQIKRLLSE

SEQ ID NO: 178 - protein, 9 aa, Felis catus
SHIKRLLSE

SEQ ID NO: 179 - protein, 9 aa, Mus musculus
RRTPSLLEQ

SEQ ID NO: 180 - protein, 9 aa, Homo sapiens
HRTPSLLEQ

SEQ ID NO: 181 - protein, 10 aa, Rattus norvegicus
EPRGSRLLVR

SEQ ID NO: 182 - protein, 19 aa, Homo sapiens
MDDQRDLISNNEQLPMLGR

SEQ ID NO: 183 - protein, 19 aa, Mus musculus
MDDQRDLISNHEQLPILGN

SEQ ID NO: 184 - protein, 20 aa, Gallus gallus
MAEEQRDLISSDGSSGVLPI

SEQ ID NO: 185 - protein, 23 aa, Danio rerio
MEPDHQNESLIQRVPSAETILGR

SEQ ID NO: 186 - protein, 23 aa, Danio rerio
MSSEGNETPLISDQSSVNMGPQP

SEQ ID NO: 187 - protein, 24 aa, Trypanosoma sp.
RPRRRTEEDELLPEEAEGLIDPQN

SEQ ID NO: 188 - protein, 19 aa, Homo sapiens
PDKHSLLVGDFREDDDTAL

SEQ ID NO: 189 - protein, 9 aa, Homo sapiens
TERERLLNF

SEQ ID NO: 190 - protein, 8 aa, Homo sapiens
VETDDLIL

SEQ ID NO: 191 - protein, 7 aa, Caenorhabditis elegans
FENDSLL

SEQ ID NO: 192 - protein, 9 aa, Saccharomyces cerevisiae
NEQSPLLHN

SEQ ID NO: 193 - protein, 8 aa, Saccharomyces cerevisiae
SEQTRLVP

SEQ ID NO: 194 - protein, 8 aa, Saccharomyces cerevisiae
EVDLDLLK

SEQ ID NO: 195 - protein, 13 aa, Homo sapiens
SFHDDSDEDLLHI

SEQ ID NO: 196 - protein, 13 aa, Bos taurus
TFHDDSDEDLLHV

SEQ ID NO: 197 - protein, 13 aa, Oryctolagus cuniculus
SFHDDSDEDLLNI

SEQ ID NO: 198 - protein, 13 aa, Gallus gallus
SFHDDSDEDLLNV

SEQ ID NO: 199 - protein, 13 aa, Homo sapiens
EESEERDDHLLPM

SEQ ID NO: 200 - protein, 13 aa, Gallus gallus
DESEERDDHLLPM

SEQ ID NO: 201 - protein, 12 aa, Homo sapiens
GYHDDSDEDLLE

SEQ ID NO: 202 - protein, 13 aa, Homo sapiens
ITGFSDDVPMVIA

SEQ ID NO: 203 - protein, 13 aa, Hydra sp.
INRFSDDEPLVVA

SEQ ID NO: 204 - protein, 13 aa, Homo sapiens
MLEASDDEALLVC

SEQ ID NO: 205 - protein, 13 aa, Homo sapiens
KNETSDDEALLLC

SEQ ID NO: 206 - protein, 12 aa, Mus musculus
WVVEAEDEPLLA

SEQ ID NO: 207 - protein, 12 aa, Homo sapiens
WVAEAEDEPLLT

SEQ ID NO: 208 - protein, 12 aa, Homo sapiens
HDDFADDISLLK

SEQ ID NO: 209 - protein, 13 aa, Mus musculus
GRDSPEDHSLLVN

SEQ ID NO: 210 - protein, 12 aa, Peromyscus maniculatus
VRCHPEDDRLLG

SEQ ID NO: 211 - protein, 13 aa, Homo sapiens
HRVSQDDLDLLTS

SEQ ID NO: 212 - protein, 13 aa, Homo sapiens
ASVSLLDDELMSL

SEQ ID NO: 213 - protein, 13 aa, Homo sapiens
ASSGLDDLDLLGK

SEQ ID NO: 214 - protein, 13 aa, Homo sapiens
VQNPSADRNLLDL

SEQ ID NO: 215 - protein, 13 aa, Homo sapiens
NALSWLDEELLCL

SEQ ID NO: 216 - protein, 13 aa, Drosophila sp.
TVDSIDDVPLLSD

SEQ ID NO: 217 - protein, 13 aa, Mus musculus
QEECPSDSEEDEG

SEQ ID NO: 218 - protein, 13 aa, Mus musculus
RDRDYDEDDEDDI

SEQ ID NO: 219 - protein, 15 aa, Mus musculus
LDETEDDELEYDDES

SEQ ID NO: 220 - protein, 10 aa, Homo sapiens
KDPDEVETES

SEQ ID NO: 221 - protein, 13 aa, Homo sapiens
HEFQDETDTEEET

SEQ ID NO: 222 - protein, 14 aa, Homo sapiens
QEKEDDGSESEEEY

SEQ ID NO: 223 - protein, 9 aa, Homo sapiens
GEDEESESD

SEQ ID NO: 224 - protein, 12 aa, Homo sapiens
GEDSDEEPDHEE

SEQ ID NO: 225 - protein, 11 aa, Homo sapiens
LEDDSDEEEDF

SEQ ID NO: 226 - protein, 9 aa, Human cytomegalovirus
KDSDEEENV

SEQ ID NO: 227 - protein, 13 aa, Herpesvirus 3
FEDSESTDTEEEF

SEQ ID NO: 228 - protein, 9 aa, Human immunodeficiency virus type 1
LEAQEEEEV

SEQ ID NO: 229 - protein, 22 aa, Saccharomyces cerevisiae
ADDLESGLGAEDDLEQDEQLEG

SEQ ID NO: 230 - protein, 12 aa, Saccharomyces cerevisiae
TEIDESFEMTDF

SEQ ID NO: 231 - protein, 18 aa, Saccharomyces cerevisiae
TEPEEVEDFDFDLSDEDH

SEQ ID NO: 232 - protein, 15 aa, Saccharomyces cerevisiae
FEIEEDDVPTLEEEH

SEQ ID NO: 233 - protein, 6 aa, Artificial Sequence
  ITIM conserved sequence
  MISC_FEATURE (1)..(1): Xaa may be Ser, Ile, Val or Leu
  misc_feature (2)..(2): Xaa can be any naturally occurring amino acid
  misc_feature (4)..(5): Xaa can be any naturally occurring amino acid
  MISC_FEATURE (6)..(6): Xaa may be Ile, Val or Leu
XXYXXX

SEQ ID NO: 234 - protein, 8 aa, Artificial Sequence
  cleavage site
DIETNPGP

SEQ ID NO: 235 - protein, 8 aa, Artificial Sequence
  cleavage site
DVETNPGP

SEQ ID NO: 236 - protein, 8 aa, Artificial Sequence
  cleavage site
DVEMNPGP

SEQ ID NO: 237 - protein, 8 aa, Artificial Sequence
  cleavage site
DVESNPGP

SEQ ID NO: 238 - protein, 8 aa, Artificial Sequence
  cleavage site
DMESNPGP

SEQ ID NO: 239 - protein, 8 aa, Artificial Sequence
  cleavage site
DVELNPGP

SEQ ID NO: 240 - protein, 8 aa, Artificial Sequence
  cleavage site
DVEENPGP

SEQ ID NO: 241 - protein, 8 aa, Artificial Sequence
  cleavage site
DIELNPGP

SEQ ID NO: 242 - protein, 8 aa, Artificial Sequence
  cleavage site
DIEQNPGP

SEQ ID NO: 243 - protein, 8 aa, Artificial Sequence
  cleavage site
DSEFNPGP

SEQ ID NO: 244 - protein, 19 aa, Artificial Sequence
  cleavage sequence
LLNFDLLKLAGDVESNPGP

SEQ ID NO: 245 - protein, 19 aa, Artificial Sequence
  cleavage sequence
LLNFDLLKLAGDVQSNPGP

SEQ ID NO: 246 - protein, 19 aa, Artificial Sequence
  cleavage sequence
LLNFDLLKLAGDVEINPGP

SEQ ID NO: 247 - protein, 19 aa, Artificial Sequence
  cleavage sequence
LLNFDLLKLAGDVEFNPGP

SEQ ID NO: 248 - protein, 19 aa, Artificial Sequence
  cleavage sequence
LLNFDLLKLAGDVESHPGP

SEQ ID NO: 249 - protein, 19 aa, Artificial Sequence
  cleavage sequence
LLNFDLLKLAGDVESEPGP

SEQ ID NO: 250 - protein, 19 aa, Artificial Sequence
  cleavage sequence
LLNFDLLKLAGDVESQPGP

SEQ ID NO: 251 - protein, 4 aa, Artificial Sequence
  ER signal peptide
KKFF

SEQ ID NO: 252 - protein, 101 aa, Homo sapiens; identical to SEQ ID NO: 21

SEQ ID NO: 253 - protein, 104 aa, Artificial Sequence
  C-terminal SH2 domain derived from SHP-1
WYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPY

SEQ ID NO: 254 - protein, 315 aa, Artificial Sequence
  truncated human CD148 intracellular domain
FEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIASGS

SEQ ID NO: 255 - protein, 4 aa, Artificial Sequence
  intracellular retention signal
HDEL